(19) United States
(12) Patent Application Publication
(10) Pub. No.: US 2023/0277762 A1
(43) Pub. Date: Sep. 7, 2023

(54) SINGLE PACKAGE AUTOMATED DRUG DELIVERY SYSTEM

(71) Applicant: INSULET CORPORATION, Acton, MA (US)

(72) Inventors: Eric DUHAMEL, Boxborough, MA (US); John D′ARCO, Wilmington, MA (US); David NAZZARO, Groveland, MA (US); James CAUSEY, Simi Valley, CA (US)

(21) Appl. No.: 18/176,969

(22) Filed: Mar. 1, 2023

Related U.S. Application Data
(60) Provisional application No. 63/315,179, filed on Mar. 1, 2022.

(51) Int. Cl. A61M 5/142

(52) U.S. Cl. CPC A61M 5/14248; A61B 5/14532

(57) ABSTRACT

Disclosed herein is a combination of an automated insulin delivery system and a continuous glucose monitor integrated into a single, wearable package. The system may use any combination of delivery methods and detection methods, wherein the delivery methods include a cannula, a microneedle array, and a transdermal patch, and wherein the detection methods include electrochemical methods, opto-fluorescent methods, and spectrographic methods.

### RELATED APPLICATIONS

This application claims the benefit of U.S. Provisional Pat. Application No. 63/315,179, filed Mar. 1, 2022, the contents of which are incorporated herein by reference in their entirety.

### BACKGROUND

Many conventional drug delivery systems are well known, including, for example, wearable drug delivery devices of the type shown in FIG. 2. The drug delivery device 102 can be designed to deliver any type of liquid drug to a user. In specific embodiments, the drug delivery device 102 can be, for example, an OmniPod® drug delivery device manufactured by Insulet Corporation of Acton, Massachusetts. The drug delivery device 102 can be a drug delivery device such as those described in U.S. Pat. No. 7,303,549, U.S. Pat. No. 7,137,964, or U.S. Pat. No. 6,740,059, each of which is incorporated herein by reference in its entirety.

A typical use for a drug delivery system is to deliver insulin. Many insulin delivery systems are capable of working with a continuous glucose monitor that communicates blood glucose values to a controller. Current systems typically require at least two separate units: a continuous glucose monitor and an insulin delivery device, that are put on the body of the user in different locations, in addition to a separate controller device. The requirement of two separate units can be cumbersome for users to install and may be uncomfortable for the user to wear. As such, this configuration is not ideal for the user’s quality of life. Additionally, some users have low body “real estate” due to scarring, age, body size, or low levels of body fat, making it challenging to wear multiple devices at one time.

In wearable, on-body devices, it is desirable to keep the wearable units as small as possible to minimize the impact to the wearer. Therefore, it would be desirable to replace the prior art insulin delivery systems having a separate insulin delivery device and glucose sensor with a single unit in which the automated drug delivery device and the glucose sensor are co-located in an adjacent or a single housing.

### DEFINITIONS

As used herein, the term “liquid drug” should be interpreted to include any drug in liquid form capable of being administered by a drug delivery device via a variety of delivery means, including, for example, insulin, GLP-1, pramlintide, glucagon, morphine, blood pressure medicines, chemotherapy drugs, fertility drugs or the like or co-formulations of two or more of GLP-1, pramlintide, and insulin. Instances of the term “insulin” as used herein should be interpreted to mean any liquid drug.

As used herein, the term “single package” should be interpreted to mean a product that may be pre-assembled at the time of manufacture and presented as one product to the end-user or comprised of two or more sub-products which are assembled at the time of use by the user. The product may also be referred to herein as a “single unit” or a “single housing”.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Various embodiments of the invention are disclosed herein in which an automated insulin delivery (AID) device and continuous glucose monitor (CGM) are co-located into one housing footprint. This eliminates the need for multiple devices which are placed in multiple locations on the body of the user, which requires the user to engage in extra installation steps. The solution herein describes various continuous glucose sensing methods or embodiments as well as insulin delivery systems where any combination of one or more of these methods or embodiments can be combined together into a single or integral device with a control algorithm to form an AID system. Combining the CGM and the means for drug delivery into a single package is referred to herein as a Single Package Insulin Delivery System (SPID) or a Single Package Automated Insulin Delivery (SPAID) System. As used herein, the term “SPAID” is meant to include non-automated embodiments (i.e., SPIDs). Any of the exemplary glucose monitoring methods listed in the table below can be mated with any of the exemplary insulin delivery methods.

| | |
|---|---|
| Glucose Monitoring Methods | Insulin Delivery Methods |
| Needle or Cannula Sensing: Electrochemical | Cannula |
| Needle or Cannula Sensing: Opto-fluorescent | Micro-Needle |
| Implanted Target: Opto-Fluorescent | Transdermal |
| Microneedle: Electrochemical | Tethered Infusion Set |
| Spectroscopy: Base Mounting Through Skin | |
| Spectroscopy: Optical Through Cannula or Needle | |

A system architecture as well as specific details around the implementation of each of the technologies is further described below. In various embodiments of the invention, the CGM may comprise an electrochemical method using a needle or flexible cannula. In other embodiments, the CGM may comprise an opto-fluorescent method using a needle or cannula or an implanted target. In yet other embodiments the CGM may comprise a spectrographic method using a needle or cannula or base monitoring through the user’s skin.

In various embodiments, the insulin infusion method may use one or more of a cannula, microneedles, a transdermal method, a tethered infusion set or any other means for insulin infusion, or any combination of the foregoing.

Any of the methods listed above can be mated together in a in a single unit or in multiple units which are physically combined at the time of use to form a single unit. A system architecture as well as specific details around the implementation of each of the technologies is further described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, like reference characters generally refer to the same parts throughout the different views. In the following description, various embodiments of the present invention are described with reference to the following drawings, in which:

FIG. 1 illustrates a functional block diagram of an exemplary system suitable for implementing the systems and methods disclosed herein.

FIG. 2 is a depiction of a prior art wearable drug delivery device.

FIGS. 3(A-B) show an embodiment wherein the glucose sensing and insulin delivery are combined into a single cannula.

FIGS. 4(A-B) show an embodiment wherein the glucose sensing and insulin delivery are combined into a single cannula, wherein the cannula has a closed-end and delivers the insulin through a series of micro-perforations.

FIGS. 5(A-B) shows an embodiment wherein insulin delivery is provided via a first cannula configured with an or cross-drilled holes and wherein the glucose sensing is provided via a second cannula.

FIGS. 6(A-B) show an embodiment wherein a two-electrode configuration is employed in which the electrochemical cell comprises one working electrode and one counter/reference electrode.

FIGS. 7(A-B) show an embodiment wherein a three-electrode configuration is employed in which the electrochemical cell comprises one working electrode, one counter electrode, and one reference electrode.

FIG. 8 shows an embodiment wherein a four-electrode configuration is employed in which the electrochemical cell comprises a main working electrode, an additional working electrode, one reference electrode, and one counter electrode.

FIG. 9 shows an embodiment employing a first cell, which comprises a first working electrode, a first counter electrode, and a first reference electrode, and a second cell, which comprises a second working electrode, a second counter electrode, and a second reference electrode.

FIG. 10 shows an embodiment in which glucose is indirectly measured at a working electrode through generated hydrogen peroxide.

FIG. 11 shows an embodiment in which glucose is indirectly measured at a working electrode through a mediator.

FIG. 12 shows an embodiment in which glucose is indirectly measured at a working electrode through a Direct Electron Transfer (DET) glucose reactive enzyme.

FIG. 13 shows an embodiment in which glucose is measured at a working electrode by direct electron transfer.

FIGS. 14(A-C) show various embodiments for a sensing element wherein the sensing electrodes are located on the exterior of the cannula.

FIGS. 15(A-C) show various embodiments for sensing cannula wherein the sensing electrodes are configured as bands wrapped around the exterior of the cannula.

FIG. 16 is a system diagram showing the components for use with an electrochemical cell method of glucose detection.

FIG. 17 illustrates a direct glucose to light method in which a receptor fluorophore is bound to an acceptor molecule and in which a glucose receptor of the acceptor molecule is not bound to glucose.

FIG. 18 shows energy excitation, energy loss and energy emission states of a receptor fluorophore when there is Förster Resonance Energy Transfer (FRET) quenching.

FIG. 19 illustrates a direct glucose to light method in which a receptor fluorophore and a receptor glucose molecule are bound to an acceptor molecule.

FIG. 20 shows energy excitation, energy loss and energy emission states of a receptor fluorophore when there is no FRET quenching.

FIG. 21 illustrates an indirect glucose to light method in which a receptor fluorophore and a receptor oxygen molecule are bound to an acceptor molecule.

FIG. 22 illustrates an indirect glucose to light method in which a receptor fluorophore is bound to an acceptor molecule and in which an oxygen receptor of the acceptor molecule is not bound to an oxygen molecule.

FIG. 23 shows one possible configuration of a glucose sensor using the opto-fluorescent sensing method employing a deployed sensing element wherein the excitation and emission lights are transmitted through the skin.

FIG. 24 shows one possible configuration of a glucose sensor using the opto-fluorescent sensing method in which a sensor coating is disposed on the surface of a cannula and further wherein the excitation and emission lights are transmitted through the skin.

FIGS. 25(A-C) show one possible configuration of a glucose sensor using the opto-fluorescent sensing method in which a sensor coating is disposed on the surface of the cannula and further wherein the excitation and emission light is transmitted via light pipes within the cannula.

FIGS. 26(A-C) show one possible embodiment of a glucose sensor using the opto-fluorescent sensing method in which multiple sensing regions are deployed as sensor coatings on the exterior surface of the sensing element to provide readings of differing concentrations of glucose.

FIGS. 27(A-B) is a variation of the embodiments of FIG. 26 (A-C) in which an additional sensing area is provided on the exterior surface of the sensing element to provide a reference oxygen level.

FIG. 28 is a system diagram showing the components for use with an opto-fluorescent method of glucose detection.

FIG. 29 is an embodiment using an implanted opto-fluorescent seed as a sensing element and further wherein the excitation and emission lights are transmitted through the skin.

FIG. 30 is an embodiment in which the glucose sensing was performed by an implanted glucose sensor which communicates glucose readings wirelessly.

FIG. 31 shows an embodiment of a microneedle-based sensing system containing two 3-electrode electrochemical cells.

FIG. 32 shows an embodiment of a hollow-microneedle-based sensing system containing a 3-electrode electrochemical cell.

FIG. 33 shows an embodiment of a hollow-microneedle-based sensing system containing an opto-fluorescent sensing array.

FIG. 34 shows an embodiment of a hollow-microneedle-based sensing system containing first and second opto-fluorescent sensing arrays.

FIG. 35 shows an embodiment of a hollow-microneedle-based sensing system containing a spectroscopy-based sensing array.

FIG. 36 shows an embodiment of a hollow-microneedle-based sensing system containing a spectroscopy-based sensing array, which allows for orientation of analyte molecules.

FIG. 37 shows a typical plot of resonated glucose energy after the glucose has been excited with near IR light energy.

FIGS. 38(A-D) show several embodiments using a microneedle sensing method, where the different embodiments show different placements of the microneedle arrays.

FIG. 39 shows an embodiment wherein the glucose sensing is performed using a spectroscopy-based method wherein the electromagnetic (EM) energy is directed through the skin

FIG. 40 shows an embodiment wherein the glucose sensing was performed using a spectroscopy-based method wherein a secondary molecule is excited with EM energy via cannula inserted into the skin.

FIG. 41 is a system diagram showing one possible limitation of a glucose sensing system using spectroscopy through a cannula.

FIGS. 42 (A-B) show two possible configurations of the tip of the cannula for the system of FIG. 41.

FIG. 43 shows an embodiment utilizing a cannula for delivery of the liquid drug into the body of the user

FIGS. 44(A-B) shows an embodiment utilizing a microneedle array for delivery of the liquid drug in the body of the user.

FIG. 45 shows an embodiment of a delivery system utilizing a microneedle array in which each microneedle is covered in a grid of individual conduction plate sections, which are individually connected to a control electronics.

FIG. 46 shows an embodiment utilizing a hollow-microneedle-based delivery system, in which diffusion pressure is used to drive the transport of insulin.

FIG. 47 shows an embodiment utilizing a hollow-microneedle-based delivery system, in which iontophoresis is used to drive the transport of insulin.

FIG. 48 shows an embodiment utilizing a hollow-microneedle-based delivery system, in which iontophoresis and sonophoresis are used to drive the transport of insulin.

FIGS. 49(A-B) show an embodiment utilizing a transdermal patch for delivery of the liquid drug into the body of the user.

FIG. 50 shows an embodiment utilizing transdermal-based delivery system, in which iontophoresis is used to drive the transport of insulin.

FIGS. 51(A-D) show several embodiments utilizing a transdermal-based delivery system, where the different embodiments show different placements of multiple pole conductors and a glucose sensing area.

FIG. 52 is a schematic of one possible implementation of a circuit used for electroporation.

FIG. 53 shows an embodiment illustrating one possible position of electroporation electrodes in an electroporation system.

FIG. 54A shows an embodiment utilizing transdermal-based delivery system, in which sonophoresis is used to drive the transport of insulin.

FIG. 54B shows an embodiment utilizing a transdermal-based delivery system, in which sonophoresis is used to drive the transport of insulin, and in which multiple ultrasonic transducers are placed in a ring around a periphery of insulin delivery port.

### DETAILED DESCRIPTION

This disclosure presents various systems, components and methods for moving a drug from a drug delivery device to a patient, such as via a needle, a cannula, a microneedle array, or transdermal patch, in combination with an analyte sensing device, such as a glucose sensor. The embodiments described herein provide one or more advantages over conventional, prior art systems, components and methods, namely, a smaller overall footprint of the combination drug delivery and sensor devices.

Various embodiments of the present invention include systems and methods for delivering an appropriate amount of medication to a user using a drug delivery device (sometimes referred to herein as a “pod”), either autonomously, or in accordance with a wireless signal received from an electronic device. In various embodiments, the electronic device may include a user device comprising a smartphone, a smart watch, a smart necklace, smart jewelry, a module attached to the drug delivery device, or any other type or sort of electronic device that may be carried by the user or worn on the body of the user and that executes an algorithm that computes the times and dosages of delivery of the medication.

For example, the user device, or more preferably, the drug delivery device itself, may execute an “artificial-pancreas” algorithm that computes the times and dosages of delivery of insulin based on sensor data, such as an analyte sensor, such as a glucose sensor. The user device, or the drug delivery device itself, may also be in communication with or integrated with one or more additional sensors, such as an accelerometer, a location sensor, a heart rate sensor, a body temperature sensor, a heart rate sensor (which may detect heart rate variability), an oxygen saturation sensor, or other sensors, that collect data on a physical attribute or condition of the user. The sensors may be disposed in or on the body of the user and may be part of the drug delivery device or may be a separate device. In exemplary embodiments, one or more analyte sensors, an example of which would be a glucose sensor, is incorporated into the drug delivery device. Additional analyte sensors may be external to the drug delivery device and in communication therewith. Outputs from the analyte sensor(s) and, if present, any additional sensors, may be used as inputs to the algorithm to compute times and dosages of drug delivery.

The drug delivery device may be in communication with the sensors in lieu of or in addition to the communication between the sensors and the user device. The communication may be direct (if, e.g., the sensors are integrated with or otherwise a part of the drug delivery device) or remote/wireless (if, e.g., the sensors are disposed in a different housing than the drug delivery device). In these embodiments, the drug delivery device contains computing hardware (e.g., a processor, memory, firmware, etc.) that executes some or all of the algorithm that computes the times and dosages of delivery of the medication without relying on a remote user device to execute the algorithm or perform such calculations.

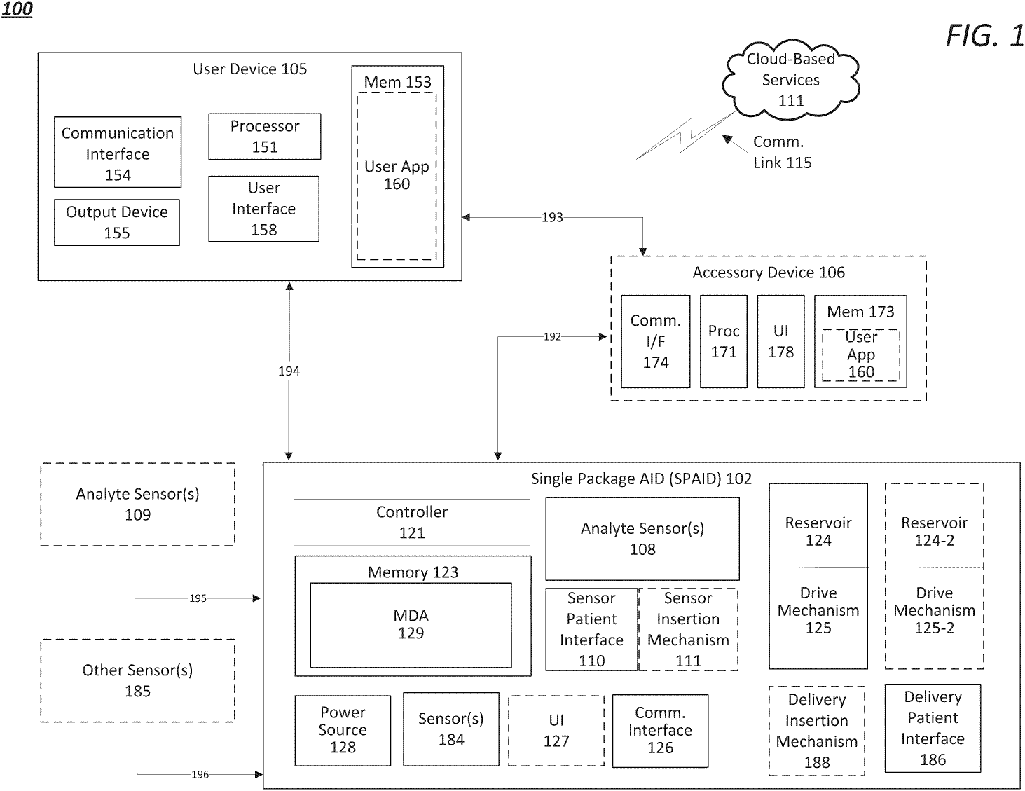

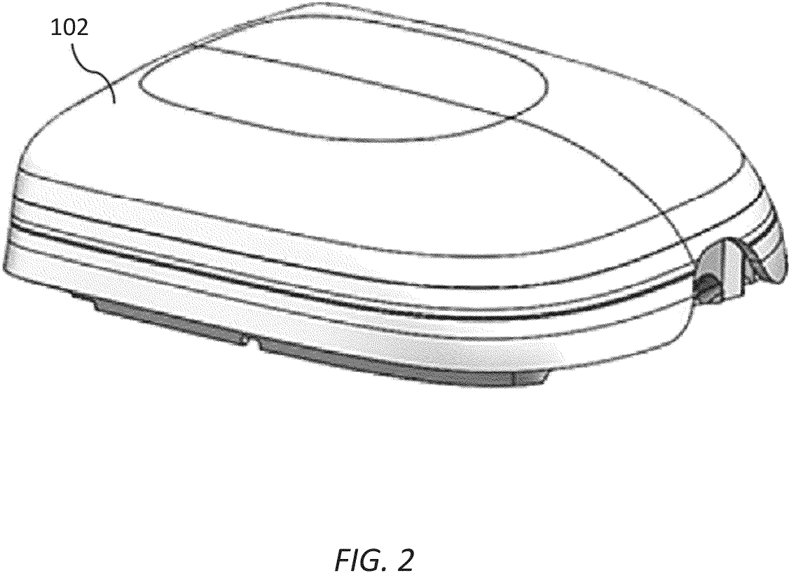

FIG. 1 illustrates a functional block diagram of an exemplary closed-loop drug delivery system 100 suitable for implementing the systems and methods described herein. The drug delivery system 100 may implement (and/or provide functionality for) a medication delivery algorithm, such as an artificial pancreas (AP) application, to govern or control the automated delivery of a drug or medication, such as insulin, to a user (e.g., to maintain euglycemia - a normal level of glucose in the blood). The drug delivery system 100 may be an automated drug delivery system that may include a drug delivery device 102 (which may be wearable or body-adhered), one or more integrated analyte sensors 108, one or more external analyte sensors 109, which may be in communication with SPAID 102 via communication link 195. (which may also be wearable or body-adhered), and a user device 105.

Drug delivery system 100, in an optional example, may also include one or more accessory devices 106, such as a primary and/or secondary smartphone, a smartwatch, a personal assistant device, an inhaler, a drug delivery pen, or the like, which may communicate with the other components of system 100 via either a wired or wireless communication links 192-193. The accessory device(s) may be used by the user or by another person, such as a parent or health care professional, for example.

### Single Package AID

In various exemplary embodiments, SPAID 102 may be configured to deliver various doses of a drug to a user in accordance with calculations performed by an artificial pancreas (AP) application or a medication delivery algorithm (MDA) 129 stored in memory 123 and executed by controller 121. Alternatively, SPAID 102 may receive instructions to deliver doses of the liquid drug from an external device, for example, user app 160 executing on a user device 105, and communicated to SPAID 102 via communication link 194.

SPAID 102 may use one or more positive displacement pumping systems in which a drive mechanism 125 operates to longitudinally translate a plunger through reservoir 124, such as to force the liquid drug through an outlet fluid port to patient interface 186. In alternate embodiments, a motor may be used to deliver the liquid drug. In an alternate embodiment, SPAID 102 may include an optional second reservoir 124-2 and second drive mechanism 125-2, which enables independent delivery of two different liquid drugs. As an example, reservoir 124 may be filled with insulin, while reservoir 124-2 may be filled with Pramlintide, GLP-1, or glucagon, for example. In some embodiments, each of reservoirs 124, 124-2 may be configured with a separate drive mechanism 125, 125-2, respectively, which may be separately controllable by controller 121 under the direction of MDA 129 or via signals received from user device 105. Both reservoirs 124, 124-2 may be connected to a common patient interface 186. The reservoirs 124, 124-2 may be configured to store drugs, medications or therapeutic agents suitable for automated delivery, such as insulin, Pramlintide, GLP-1, co-formulations of insulin and GLP-1, glucagon, morphine, blood pressure medicines, chemotherapy drugs, fertility drugs or the like.

SPAID 102 may be configured with one or more integrated analyte sensors 108, or one or more external analyte sensors 109, which may be, for example, a continuous glucose monitor. The analyte sensor(s) 108, 109 may be configured to detect multiple different analytes, such as glucose, lactate, ketones, uric acid, sodium, potassium, alcohol levels, or the like, and output results of the detections, such as measurement values or the like. The analyte sensor(s) 108, 109 may, in an exemplary embodiment, be controllable by MDA 129 to measure a blood glucose value at a predetermined time interval, such as every 5 minutes, every 1 minute, or the like, or in an ad hoc manner. Readings from analyte sensor 108 may be communicated to MDA 129 for use in determining the quantity and timing of the delivery of doses of the liquid drug to the user. In addition, or alternatively, readings from analyte sensor(s) 108, 109 may be communicated to user device 105 via communication link 194 for use by user app 160. Analyte sensor 108 is coupled to a sensor patient interface 110, which serves to interface with the body of the patient to obtain the required information necessary to deliver the readings. Various embodiments of the invention include different types of analyte sensor(s) 108, 109 and various types of sensor patient interfaces 110, which will be described in detail herein. In descriptions herein, references to integrated analyte sensor(s) 108 would generally apply to external analyte sensors 109 as well. In some embodiments, a sensor insertion mechanism 111 may be provided for insertion of sensor patient interface 110. In some embodiments, the sensor patient interface may be, for example, a cannula, needle or other sensing element. The sensor insertion mechanism 111 may comprise, in some embodiments, an actuator that inserts the sensor patient interface 110 sensing element under the skin of the user.

Although the analyte sensor 108 is depicted in FIG. 1 as being integral with drug delivery device 102, in various non-SPAID embodiments, the analyte sensor 108 and drug delivery device 102 may be housed in separate housings and may be applied to the body of the user in different locations. In another embodiment, one or more analyte sensors 108 and sensor patient interface 110 may be housed in a separate housing which is attachable to the housing of SPAID 102.

SPAID 102 includes a delivery patient interface 186 for interfacing with the user to deliver the liquid drug(s) from reservoir 124 (or in the case of two reservoirs, from either or both of reservoirs 124, 124-2). The delivery patient interface 186 may be, for example, a needle or cannula for delivering the drug into the body of the user (which may be done subcutaneously, intraperitoneally, or intravenously). Alternatively, delivery patient interface 186 may be one or more arrays of microneedles. In yet other alternate embodiments, delivery patient interface 186 may be a transdermal method of delivery. The various types of delivery patient interfaces 186 will be discussed in detail later herein. In the case wherein the delivery patient interface 186 is a cannula/needle, SPAID 102 further includes delivery insertion mechanism 188 for inserting the cannula into the body of the user, which may be integral with or attachable to SPAID 102. The delivery insertion mechanism 188 may comprise, in one embodiment, an actuator that inserts a needle and cannula under the skin of the user and thereafter retracts the needle, leaving the cannula in place. Delivery insertion mechanism 188 may be combined with sensor insertion mechanism 111 such that one mechanism delivers both a sensor and a cannula/needle in a single step or in multiple steps.

SPAID 102 may be optionally configured with a user interface 127 providing a means for receiving input from the user and a means for outputting information to the user. User interface 127 may include, for example, light-emitting diodes, buttons on a housing of SPAID 102, a sound transducer, a micro-display, a microphone, an accelerometer for detecting motions of the device or user gestures (e.g., tapping on a housing of SPAID 102) or any other type of interface device that is configured to allow a user to enter information and/or allow SPAID 102 to output information for presentation to the user (e.g., alarm signals or the like).

In one embodiment, SPAID 102 includes a communication interface 126, which may be a transceiver that operates according to one or more radio-frequency protocols, such as Bluetooth®, Wi-Fi, near-field communication, cellular, or the like. The controller 121 may, for example, communicate with user device 105, accessory device 106 and cloud-based service 111 via the communication interface 126.

In some embodiments, SPAID 102 may be provided with one or more other integrated sensors 184 and one or more other external sensors 185, which may be in communication with SPAID 102 via communication link 196. The sensors 184, 185 may include, for example, one or more of an accelerometer, a location sensor, a heart rate sensor, a body temperature sensor, a heart rate sensor (which may detect heart rate variability), an oxygen saturation sensor, or other sensors, a pressure sensor, a power sensor, or the like that are communicatively coupled to the controller 121 and provide various signals to MDA 129 or user app 160. For example, a pressure sensor may be configured to provide an indication of the fluid pressure detected in a fluid pathway between the patient interface 186 and reservoir 124 for purposes of identifying an occlusion or an improper insertion by delivery insertion mechanism 188. The pressure sensor may be coupled to or integral with the insertion mechanism 188 for inserting the patient interface 186 into the user. In an example, the controller 121 may be operable to determine a rate of drug infusion based on an indication of the fluid pressure. The rate of drug infusion may be compared to an infusion rate standard or threshold, and the comparison result may be usable in determining an amount of insulin onboard (IOB), a total daily insulin (TDI) amount, or whether an occlusion has occurred.

SPAID 102 further includes a power source 128, such as a battery, a piezoelectric device, an energy harvesting device, or the like, for supplying electrical power to all components of SPAID 102. Power source 128 may be removable or rechargeable.

SPAID 102 may be configured to perform and execute processes required to deliver doses of the liquid drug to the user without input from the user device 105 or the optional accessory device 106. As explained in more detail, MDA 129 may be operable, for example, to determine an amount of insulin to be delivered, insulin-on-board (IOB), insulin remaining in reservoir 124, and the like and to cause controller 121 to activate drive mechanism(s) 125 (125-2) to deliver the liquid drug from reservoir(s) 124 (124-2). MDA 129 may take as input data received from the analyte sensor(s) 108, 109 or other sensors 184, 185.

SPAID 102 may be a wearable device and may be attached to the body of a user, such as a diabetic user, at an attachment location and may deliver any therapeutic agent, including any drug or medicine, such as insulin or the like, to a user at or around the attachment location. A surface of SPAID 102 may include an adhesive to facilitate attachment to the skin of a user. The adhesive may be a pad that is spot-welded to SPAID 102 for attachment thereto.

### User Device

The user device 105 may be a computing device such as a smartphone, a tablet, a personal diabetes management (PDM) device, a dedicated diabetes therapy management device, a wearable device, or the like. In an example embodiment, user device 105 may include a processor 151, device memory 153, a user interface 158, and a communication interface 154. The processor 151 may execute processes based on software stored in device memory 153, such as user application 160, to manage a user’s blood glucose levels and for controlling the delivery of the liquid drug to the user via SPAID 102, as well for providing other functions, such as calculating carbohydrate-compensation dosage, a correction bolus dosage and the like as discussed below. The user device 105 may be used to program, adjust settings, and/or control the operation of SPAID 102 as well as the optional smart accessory device 106.

The user app 160 may be an application that is operable to calculate doses of a drug for delivery to the user based on information received from the analyte sensor 108 of SPAID 102, other sensors 184, the cloud-based services 111 and/or the user device 105 or optional accessory device 106. The memory 153 may also store software to, for example, operate the user interface 158 (e.g., a touchscreen device, a camera, etc.), the communication interface 154, and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 is an AP application, the processor 151 may also be configured to execute a diabetes treatment plan (which may be stored in a memory) that is managed by user app 160. In addition to the functions mentioned above, when user app 160 is an AP application, it may further provide functionality to determine a carbohydrate-compensation dosage, a correction bolus dosage and determine a basal dosage according to a diabetes treatment plan. In addition, as an AP application, user app 160 provides functionality to output signals to the SPAID 102 via communications interface 194 to deliver the determined bolus and basal dosages.

User device 105 may be further provided with one or more output devices 155 which may be, for example, a speaker or a vibration transducer, to provide various signals to the user under the control of user app 160, for example, alarms. User app 160 may also have access to other facilities of the user device 105, for example, a GPS unit, a clock, a camera, etc., which may provide information to user app 160 which may be useful in the calculation dosing information of the liquid drug.

### Accessory Device

Optional accessory device 106 may be, a wearable device, for example, a smart watch (e.g., an Apple Watch®), smart eyeglasses, smart jewelry, a GPS-enabled wearable, a wearable fitness device, smart clothing or the like. Additionally, accessory device 106 may be an inhaler or a drug delivery pen capable of communicating information regarding quantities of insulin delivered to the user to other components of SPAID 102. MDA 129 may use this information to adjust the IOB and thereafter to adjust bolus and basal delivery accordingly.

Similar to user device 105, the accessory device 106 may also be configured to perform various functions including controlling SPAID 102. For example, the accessory device 106 may include a communication interface 174, a processor 171, a user interface 178 and a memory 173. The user interface 178 may be a graphical user interface presented on a touchscreen display of the smart accessory device 106. The memory 173 may store programming code to operate different functions of the smart accessory device 106 as well as an instance of the user app 160, or a pared-down version of user app 160 with reduced functionality. In some instances, accessory device 106 may also include sensors of various types. In some embodiments, user app 160 executing on accessory device 106 may act in concert with user app 160 executing on user device 105 to accept input from the user or to provide alerts or alarms to the user.

### Cloud-Based Services

Drug delivery system 100 may communicate with or receive data or services from cloud-based services 111. Services provided by cloud-based services 111 may include data storage that stores personal or anonymized data, such as blood glucose measurement values, historical IOB or TDI, prior carbohydrate-compensation dosage, and other data. In addition, the cloud-based services 111 may process anonymized data from multiple users to provide generalized information related to TDI, insulin sensitivity, IOB and the like. The communication link 115 that couples the cloud-based services 111 to the respective devices 102, 105, and 106 of system 100 may be a cellular link, a Wi-Fi link, a Bluetooth® link, or a combination thereof. In some embodiments, cloud-based services 111 may include cloud-based processors which may be used by drug delivery system 100 to replace or supplement processors 151 or 171, or controller 121.

### Communication Links

The wireless communication links 115 and 192-196 may be any type of wireless link operating using known wireless communication standards or proprietary standards. As an example, the wireless communication links 192-194 may provide communication links based on Bluetooth®, Zigbee®, Wi-Fi, a near-field communication standard, a cellular standard, or any other wireless protocol via the respective communication interfaces 126, 154 and 174.

### Calibration

A drug delivery system 100 using a combined SPAID 102 combining the insulin/drug delivery system and a CGM may require calibration to maintain accurate monitoring and subsequent control of the user’s blood glucose levels. System 100 may be capable of accepting reference blood glucose values taken by the user and entered for use by system 100 via, for example user app 160 running on user device 105 or on accessory device 106.

In addition to having the user enter blood glucose reference values manually, another possible method for calibrating may be an active SPAID 102 to communicate with an additional CGM device that the user may be currently using or may apply to the user’s body anew. If the user is actively wearing an additional CGM device or smart watch device, for example, accessory device 106, it may be possible for SPAID 102 to communicate with that device at startup or periodically throughout its use cycle to gather reference blood glucose values to establish a startup reference calibration and/or check or confirm its accuracy during runtime.

An additional use model for SPAID 102 would be to have the user install a new SPAID 102 prior to the end of life of the previous SPAID 102. The system 100 could warn or inform the user to install the next SPAID 102 for use, as the prior SPAID 102 is approaching the end of its usable life. At that time, the prior SPAID 102 could communicate either directly via a wireless communication method or indirectly using user device 105 or accessory device 106 as an intermediary between the two SPAIDs. The two SPAIDs would then be worn and both CGMs in the SPAIDs would be active for a short overlapping period of time long enough to calibrate the new CGM. The prior SPAID 102 would then share its current CGM data with the new SPAID 102 and calibrate the new SPAID 102 CGM and confirm its accuracy prior to ending its use. The new SPAID 102 would only begin to administer insulin once it has received confirmation that the prior SPAID 102 has been removed and or disabled and can no longer deliver insulin. This is necessary to prevent two SPAIDs from delivering insulin together and potentially driving the user’s blood glucose levels too low.

### Operational Example

In an operational example, user application 160 implements a graphical user interface that is the primary interface with the user and is used to collect information necessary to operate SPAID 102 (e.g., age, weight, or other information), program basal profiles and provide for use of a bolus calculator for operation in a manual mode, initiate SPAID 102, trigger insertion of needle/cannula or delivery patient interface 186 using delivery insertion mechanism 188, trigger insertion of sensor patient interface 110 using sensor insertion mechanism 111, and/or program settings specific for operation in an automated mode (e.g., a closed-loop mode or a hybrid of manual and closed-loop where the user can still deliver some instructions or input (e.g., carbohydrate count or delivery of a correction bolus) to SPAID 102 via user device 105 or accessory device 106).

User app 160, provides a graphical user interface 158 that allows for the use of large text, graphics, and on-screen instructions to prompt the user through the set-up processes and the use of system 100. By way of example, user app 160 may also be used to wake up SPAID 102, deliver sensor patient interface 110, deliver delivery patient interface 186, prime SPAID 102 such as by removing air from a fluid delivery path, program the user’s custom basal insulin delivery profile, check the status of SPAID 102, initiate bolus doses of insulin such as correction boluses or meal boluses, make changes to a patient’s insulin delivery profile, handle system alerts and alarms, and allow the user to switch between automated mode and manual mode. User app 160 may also be configured to determine dosing information and provide dosing information to SPAID 102 or provide information to SPAID 102 that allows MDA 129 to determine the proper dosing of the liquid drug.

User app 160 may be configured to operate in a manual mode in which user app 160 may control the delivery of insulin at programmed basal rates and programmed or manual bolus amounts with the option to set temporary basal profiles. Controller 121 of SPAID 102 may also have the ability to function as a sensor-augmented pump when user app 160 is operating in manual mode, using sensor glucose data provided by the analyte sensor 108 to populate the bolus calculator.

User app 160 may configured to operate in an automated mode in which user app 160 supports the use of multiple target blood glucose values. For example, in one embodiment, target blood glucose values can range from 110-150 mg/dL, in 10 mg/dL increments, in 5 mg/dL increments, or other increments, but preferably 10 mg/dL increments. The experience for the user will reflect current setup flows whereby the healthcare provider assists the user to program basal rates, glucose targets and bolus calculator settings. These in turn will be used by the user app 160 as dosing parameters. The dosing parameters may be adapted over time based on the total daily insulin (TDI) delivered by SPAID 102. A temporary hypoglycemia protection mode may be implemented by the user for various time durations in automated mode. With hypoglycemia protection mode, the algorithm of user app 160 reduces insulin delivery and is intended for use over temporary durations when insulin sensitivity is expected to be higher, such as during exercise.

The user app 160 (or MDA 129) may control SPAID 102 to provide periodic insulin micro-boluses based upon past glucose measurements and/or a predicted glucose over a prediction horizon (e.g., 60 minutes). Optimal post-prandial control may require the user to give meal boluses in the same manner as current pump therapy, but normal operation of the user app 160 or MDA 129 will compensate for missed meal boluses and mitigate prolonged hyperglycemia. The user app 160 or MDA 129 uses a control-to-target strategy that attempts to achieve and maintain a set target glucose value, thereby reducing the duration of prolonged hyperglycemia and hypoglycemia.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value and IOB, which is estimated by user app 160 or MDA 129 taking into account any manual bolus and insulin delivered by the algorithm.

### Description of Specific Embodiments

In various embodiments of SPAID 102, different types of patient interfaces may be used to deliver the liquid drug and to perform the required sensing to determine the correct dosing information for the liquid drug. In some embodiments, the delivery and sensing mechanisms may be combined into a single patient interface, while in other embodiments, the delivery and sensing mechanisms may use separate patient interfaces. Additionally, different mechanisms may be used to deliver the liquid drug and different methods may be used to perform the required sensing.

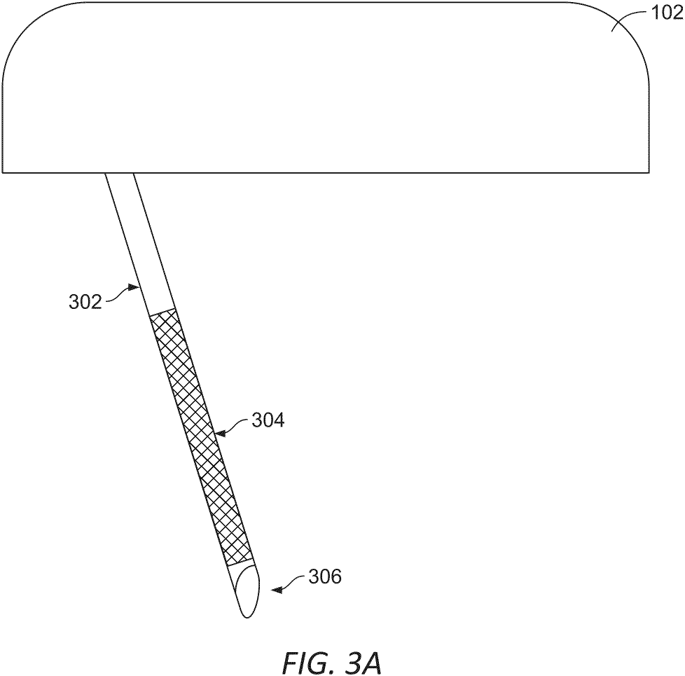

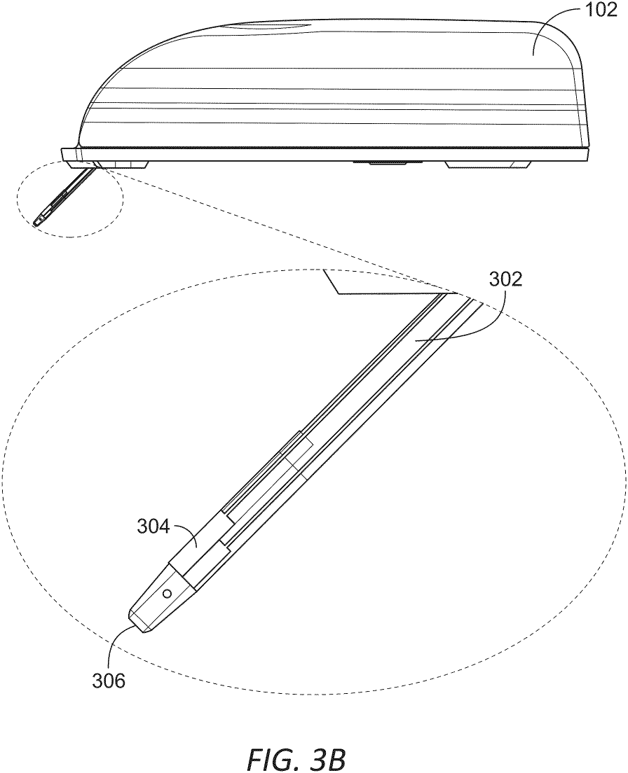

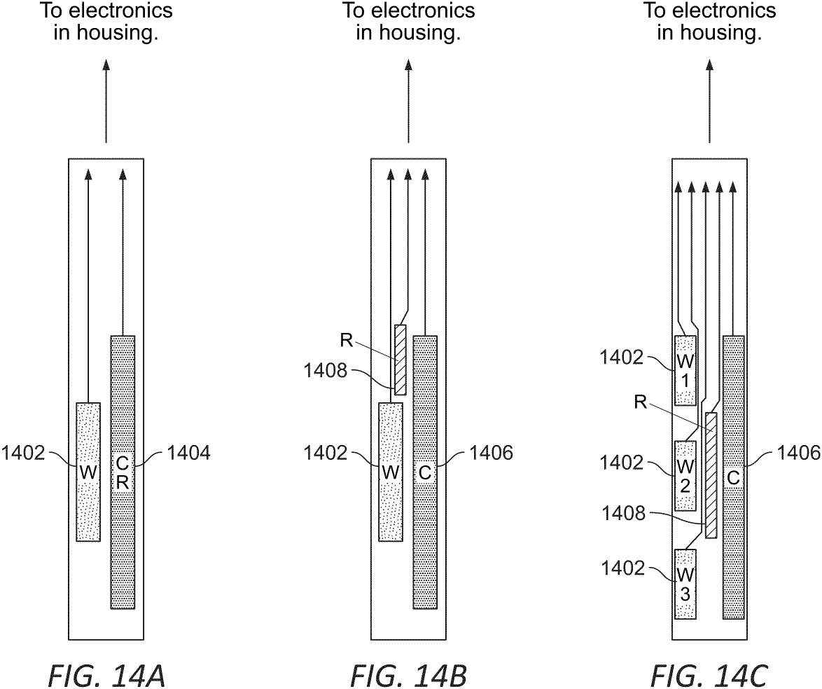

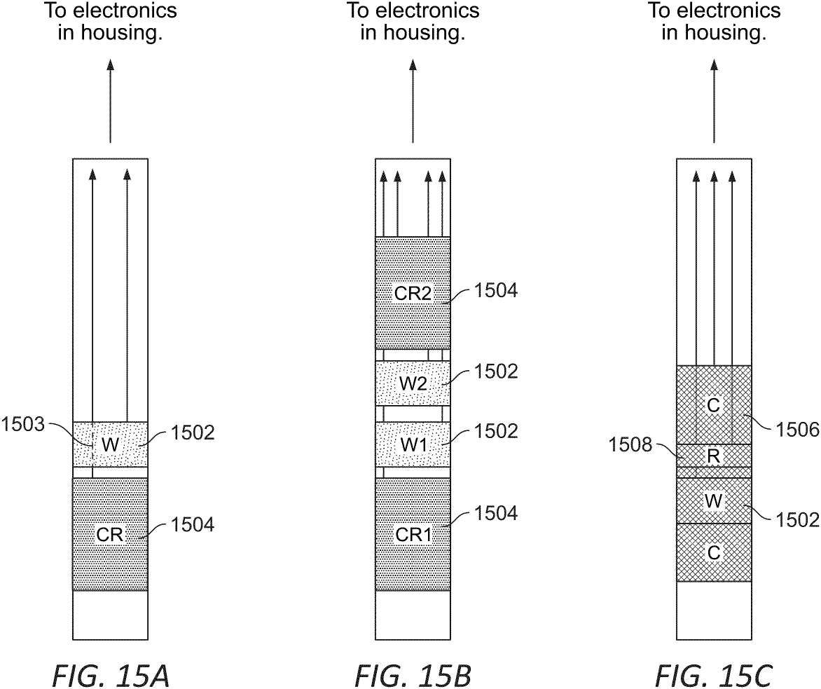

In one embodiment, shown in schematic form in FIG. 3A, glucose sensing and insulin delivery are combined in a single cannula 302. Cannula 302 is provided with a glucose sensing area 304 and the liquid drug is delivered through a lumen defined in the cannula and discharged through an open tip 306 of cannula 302. Glucose sensing area 304 can be, for example, any of the embodiments shown in FIGS. 14(A-C) and FIGS. 15(A-C). FIG. 3B shows a concept image of this embodiment, showing an enlarged view of the cannula 302 in the inset. Distal end of cannula 302 can taper to a smaller diameter at the distalmost end and may comprise a cross drilled hole through which the liquid drug may be delivered in addition to delivery through the open tip 306.

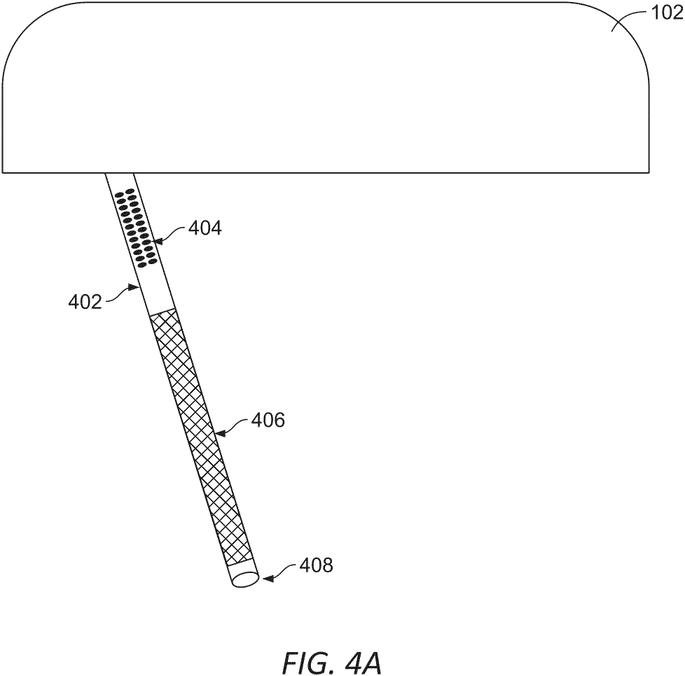

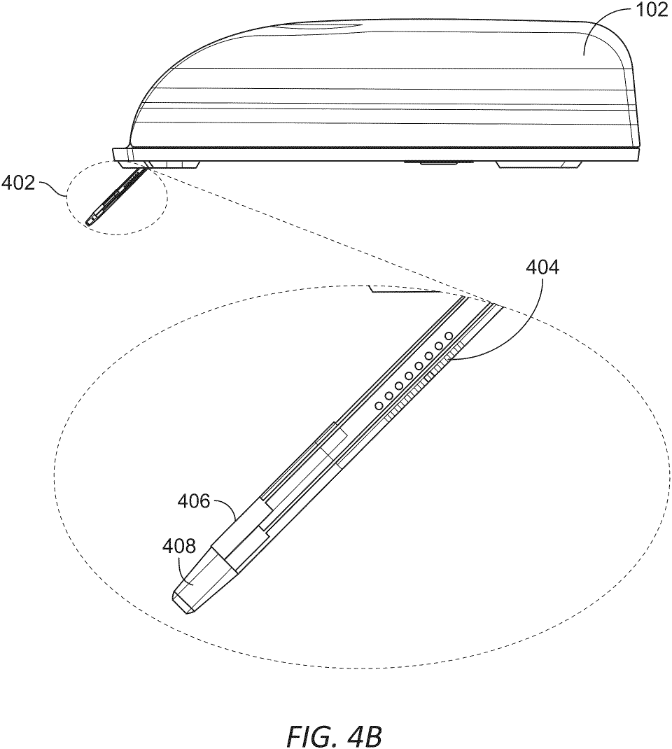

A variation on the single cannula configuration is shown in schematic form in FIG. 4A in which cannula 402 has a closed end 408 but is provided with a series of perforations 404 for delivery of the liquid drug. In this variation, cannula 402 is also provided with glucose sensing area 406. In some embodiments wherein delivery and sensing are provided on the same cannula, it may be desirable to deliver insulin at the epidermal or dermal layer while the sensing portion of the cannula is disposed subcutaneously.

Glucose sensing area 406 can be, for example, any of the embodiments shown in FIGS. 14(A-C) and FIGS. 15(A-C). FIG. 4B shows a concept image of this embodiment, showing an enlarged view of the cannula 402 in the inset. Note that cannula 402 shown in FIGS. 4A, 4B may be at any angle with respect to the bottom of SPAID 102. In some embodiments provided with both a delivery cannula and a sensing cannula or needle, it may be desirable to maximize the distance between the delivery cannula and sensing needle and, as such, these needles may be provided at angles directed to achieving this purpose.

The variation shown in FIGS. 3A-4B using a single cannula have the advantage of reducing the possible number of insertions into the body of the patient.

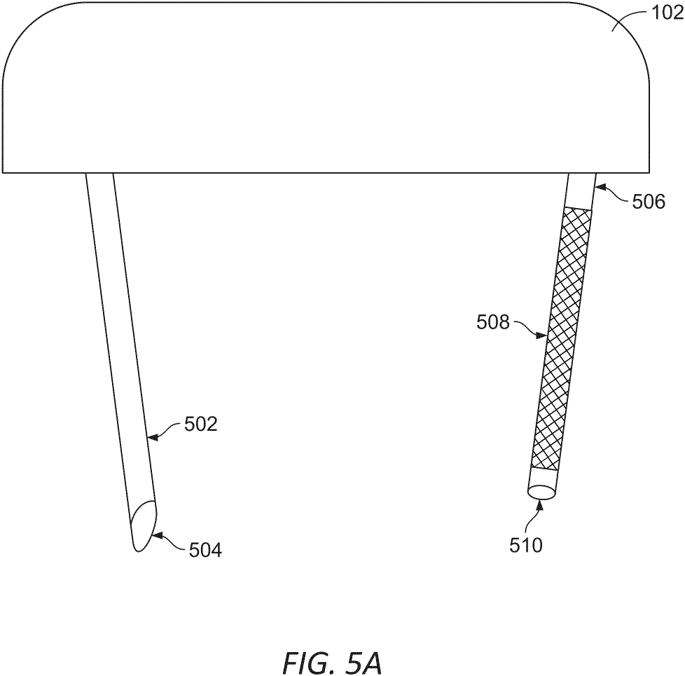

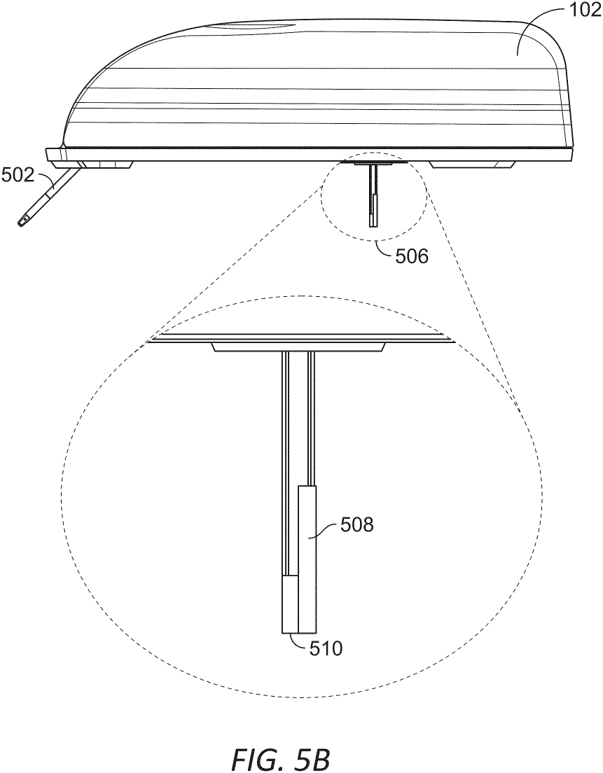

In an alternative embodiment shown in schematic form in FIG. 5A, multiple patient interfaces are inserted into the user. Cannula 502 is configured with an open end 504 for the delivery of the liquid drug, while cannula 506 is configured with a closed end 510 and a glucose sensing area 508 for sensing of glucose levels of the user. Distal end of cannula 502 can taper to a smaller diameter at the distalmost end, and may comprise a cross drilled hole through which the liquid drug may be delivered in addition to delivery through the open tip 504. FIG. 5B shows a concept image of this embodiment, showing an enlarged view of the cannula 506 in the inset.

In the embodiments shown in FIGS. 3A-5B the cannulas may be housed in SPAID 102 and inserted into the body of the user during the installation process of SPAID 102 or at any time throughout the use cycle as needed to address possible sensor fouling and site occlusions or simply sensor aging to allow the SPAID 102 to remain in the same location on the user for extended time.

To extend the life of SPAID 102 once installed onto the body of the user, it may be possible to provide multiple or additional cannula or sensing element delivery mechanisms to “fire” or be self-installing should the initially-inserted cannula become damaged or inoperative. The insertion mechanisms may be identical and may be used to insert additional delivery patient interfaces or sensor patient interfaces. The insertion/delivery mechanism may comprise a module within SPAID 102 that may be replaceable.

Furthermore, any additional installation mechanisms could install additional sensing elements that are either standalone or constructed directly onto the liquid drug delivery cannula. The additional sensors would be installed under the skin at a new site under the base of SPAID 102 and would begin to warm up once installed. After the warm-up period, the existing sensing device can be used to calibrate the newly installed device. Once the new sensor and cannula are deemed active and accurately calibrated, the original electrode and cannula will be retracted and disabled.

For cannulas delivering the liquid drug, a priming process to ensure no air is in the cannula at the time of installation may be performed prior to the installation of a delivery cannula of a combination delivery and sensing cannula, or prior to adhering SPAID 102 to the skin of the user. A pump for the cannula is activated inside of SPAID 102 and operated for a fixed period to push a fixed volume of the liquid drug through the cannula to remove any air previously therein. The amount pushed should at least equal the volume of the cannula tubing from the reservoir to the tip. A collection area or absorbent pad may be located inside of the housing of SPAID 102 to collect and absorb any excess liquid drug that may escape the cannula tip during this process prior to installation into the skin.

To support the additional sensor and cannula installation mechanisms, a retraction mechanism may be used to remove the initially installed cannula to prevent inadvertent administering of the liquid drug once the additional cannula and sensor are installed. In one embodiment, a dual spring system may be provided. One spring is used to initially install the device into the skin along a guide track with the sensor and cannula mounted onto the track using shuttle carrier device. Once the initial spring has fired it is disconnected from the drive path. A second spring retracts the sensor and cannula from the body by applying force to the shuttle carrier device in the opposite direction without the need to recompress the initial installation spring.

In a variation of the described embodiments, the same installation system may be capable of retraction and reinsertion into the body. The process of retraction would create a wiping motion along a dry pad or other chemical based cleaning surface capable of removing body fluids and other sensor fouling components to clean and restore sensor performance. In addition, the cannula may be wiped or cleaned as well to remove occlusion components. While retracted, the cannula could push additional insulin into a collection pad to force occlusion materials through the cannula. One possible implementation would be to use a dual spring system with mechanisms for enabling and disabling spring engagement between the installation spring and retraction springs to prevent one from applying force to the other. Alternatively, a motor driven screw drive mechanism may be employed to draw back the sensor slowly along a guide track. This system would also serve to rewind/reload the initial installation spring so it can be re-used to fire/reinstall the sensor and cannula.

### Electrochemical Sensing Methods

Electrochemical sensing can be used to sense glucose and other analytes in the various drug delivery system described herein. In addition, sensing can be performed by single or multiple electrochemical cells to enhance reliability, accuracy, endurance and/or overall performance. Moreover, electrochemical cells can be configured for use in any suitable electrochemical glucose sensing method, including those described in further detail below.

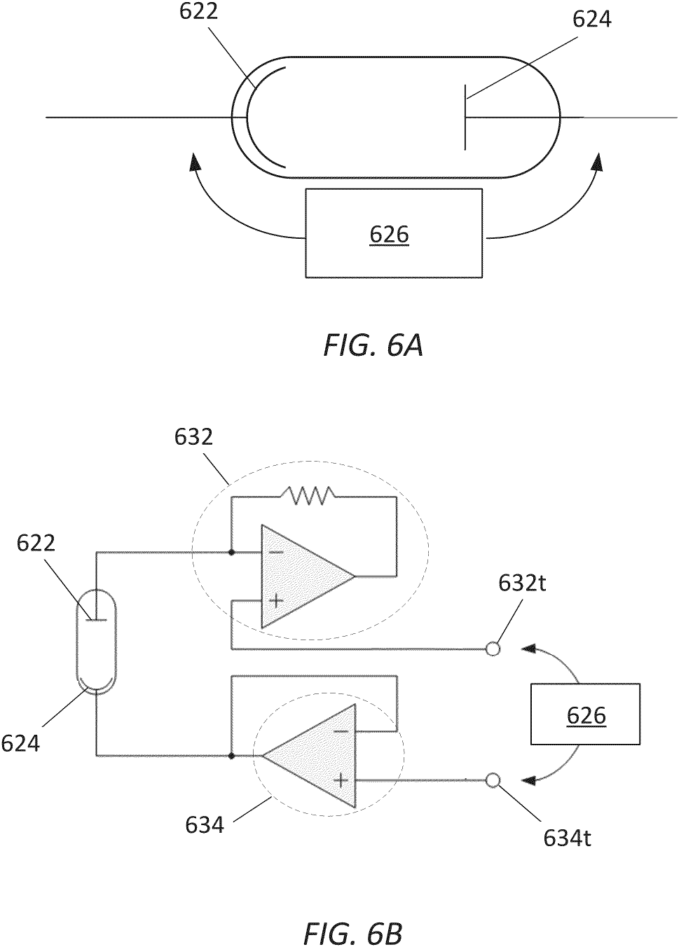

Various electrode configurations may be employed. In some embodiments, and with reference to FIGS. 6A-6B, a two-electrode configuration may be employed in which the electrochemical cell comprises one working electrode 622 and one counter/reference electrode 624. The electrodes may be operated with either a positive or a negative excitation voltage 626 to produce either reduction or oxidation reactions at the working electrode 622. In positive excitation mode, a positive voltage is applied to an input terminal 632t of a working amp 632 relative to an input terminal 634t of a counter/reference amp 634 to create a positive excitation voltage and drive a reduction reaction. In negative excitation mode, a negative voltage is applied to the input terminal 632t of the working amp 632 relative to the input terminal 634t of the counter/reference amp 634 to create a negative excitation voltage and drive an oxidation reaction. The excitation voltage 626 may be, for example, fixed, varying or pulsing. Exemplary excitation voltages may range, for example from +600 mV to - 600 mV, among other possible values. The resulting electrical current produced by the excitation voltage may be measured at either a working electrode terminal or a counter/reference terminal.

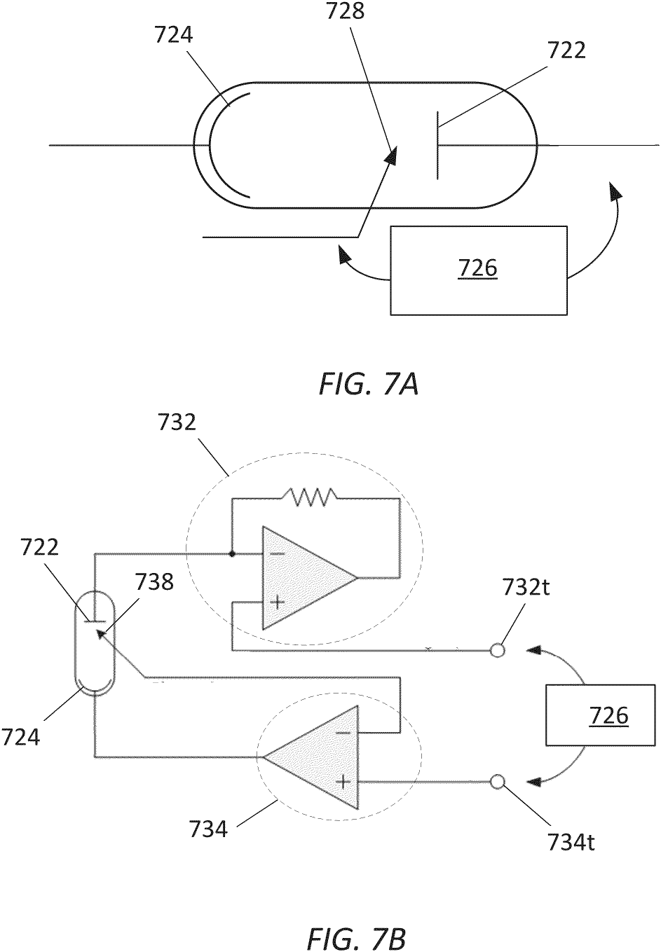

In some embodiments, and with reference to FIGS. 7A-7B, a three-electrode configuration may be employed in which the electrochemical cell comprises one working electrode 722, one counter electrode 724, and one reference electrode 728. As with the preceding two-electrode configuration, the electrochemical cell may be operated with either positive or negative excitation voltages 726 to produce either reduction or oxidation reactions, respectively, at the working electrode 722. In positive excitation mode, a positive voltage is applied to an input terminal 732t of a working amp 732 relative to an input terminal 734t of a counter/reference amp 734 to create a positive excitation voltage and drive a reduction reaction. In negative excitation mode, a negative voltage is applied to the input terminal 732t of the working amp 732 relative to the input terminal 734t of the counter/reference amp 734 to create a negative excitation voltage and drive an oxidation reaction. The excitation voltage 726 may be, for example, fixed, varying or pulsing. The excitation voltage 726 may be established between the working electrode 722 and the reference electrode 728 with the counter electrode 724 used to servo positively and negatively to ensure that the excitation voltage 726 is maintained between the working and reference electrodes 722, 728. Exemplary excitation voltages may range, for example from +600 mV to - 600 mV, among other possible values. The resulting electrical current produced by the excitation voltage may be measured at either a working electrode terminal or a counter terminal. No significant current flows through the reference electrode terminal as it is only being used to “sense” and servo the voltage in the reaction to maintain the proper excitation voltage.

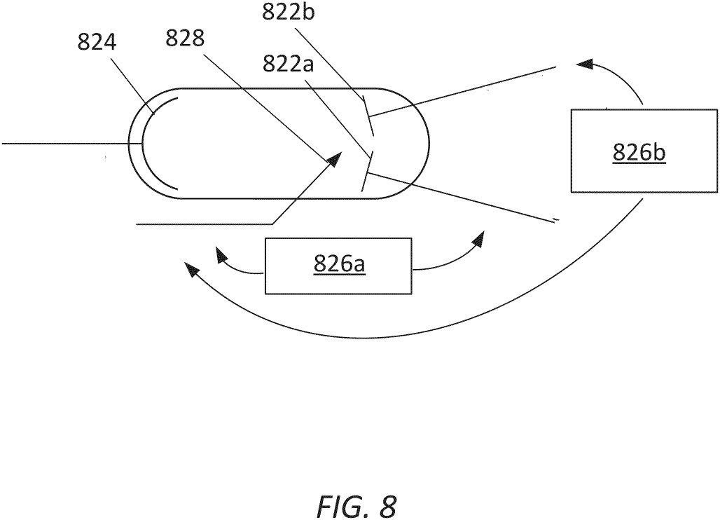

In some embodiments, and with reference to FIG. 8, a four-electrode configuration may be employed in which the electrochemical cell comprises a main working electrode 822a, an additional working electrode 822b, one reference electrode 828 and one counter electrode 824. The electrodes may be operated as described in the three-electrode configuration as described above, with the addition of a fourth electrode constructed as the additional working electrode 822b. The additional working electrode 822b may be excited at an excitation voltage 826b that is higher, lower or the same as an excitation voltage 826a associated with the main working electrode 822a. The additional working electrode 822b may have additional chemical components added to it and serve as a possible “blanking” electrode to measure background interferences present in the main working electrode signal. The additional chemical components may be any component that negates the resulting signal created by the reaction with the target analyte. In the case of glucose as a target analyte, an enzyme, e.g., glucose oxidase (GOX)or glucose dehydrogenase (GDH) is used to react with glucose to form hydrogen peroxide which is subsequently measured at the working electrode. The blanking electrode may be coated with catalase, which is a natural consumer of hydrogen peroxide and thus will prevent the blanking electrode from receiving the hydrogen peroxide that is created when glucose interacts with GOX. The removal of the peroxide will allow the blanking electrode to react to and only measure the background interferences. This “blanking” signal may then be subtracted from the main working electrode signal by the electrical system either in an analog or digital manner or by software resulting in only a pure glucose/GOX reaction signal. The additional working electrode current flows through the counter electrode terminal and its voltage can be established with respect to the reference electrode terminal.

An additional use for the additional working electrode 822b could be for “cleaning” the first working electrode if it becomes fouled or coated by unwanted chemical species. Examples of such species include phenols, amino acids, neurotransmitters, proteins, and other biomolecules, including whole or fragmented cells, DNA molecules, and/or RNA molecules, which over time can form a layer on the working electrode and reduce its sensitivity and performance. The additional working electrode 822b may initially be dormant and electrically disconnected and subsequently be activated as needed (e.g., after fouling or after a time deemed sufficient for fouling to occur) at a voltage that is capable of pulling the unwanted chemical species off of the main working electrode 822a onto itself. This activation may occur repeatedly at programmed intervals, and/or upon detection of reduced sensitivity of the working electrode. In this embodiment, the additional working electrode 822b is sufficiently close to the main working electrode 822a to ensure the cleaning is rapid (less than 10 minutes) and complete. In general, the working electrode 822b and main working electrode 822a should be as close to each other as the manufacturing process will allow and should ideally be less than 1 mm apart. Once the unwanted chemical species are removed the system is returned to normal operation. This process may repeat as needed.

In a further embodiment, the additional working electrode 822b may be used as a backup electrode for the main working electrode 822a, for example, in the event that the main working electrode 822a becomes fouled. For instance, additional working electrode 822b may initially be dormant and subsequently be activated and used as the primary analyte measurement electrode, and the fouled working electrode 822a disabled. This could be repeated “N” number of times for as many additional working electrodes as are added to the system. In such a configuration of the system, the working electrodes should ideally be sufficiently separated to be electrically isolated from other electrodes.

Any number of possible electrochemical cell configurations may be established as well, including those that include multiple electrochemical cells. Any of these multiple cell configurations may comprise two-, three- or four-electrode cells. In addition, multiple cell configurations may be established in which the electrochemical cells use the same electrical system ground. Alternatively, multiple electrochemical cells may be established in which each of the electrochemical cells uses an isolated or floating ground system so as to prevent “cross-talk” between two adjacent electrochemical cells. For example, this may be especially beneficial when creating a blanking cell to prevent the working electrode terminals from attempting to pass current into adjacent reference or counter electrical terminals.

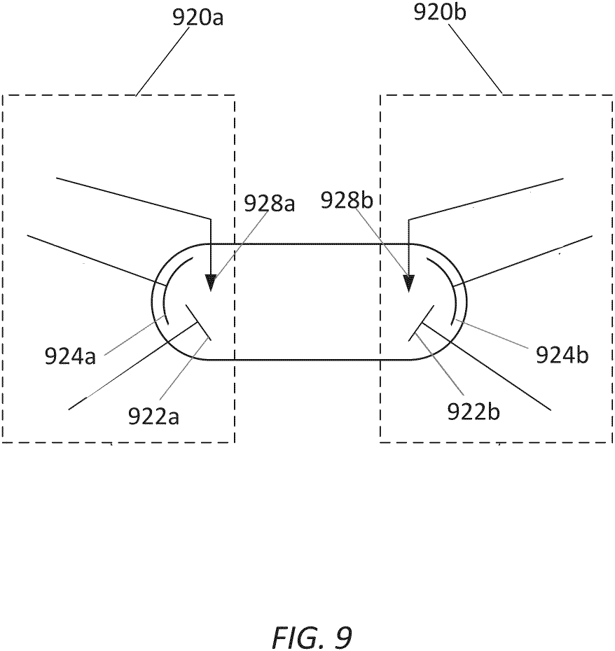

In the embodiment shown in FIG. 9, a first cell 920a, which comprises a first working electrode 922a, a first counter electrode 924a, and a first reference electrode 928a, may be constructed with suitable chemicals for measuring the desired analyte with interferences. A second cell 920b, which comprises a second working electrode 922b, a second counter electrode 924b, and a second reference electrode 928b, may be constructed with suitable chemicals for measuring just the interferences. As previously noted, an example is in the case of a glucose measuring system where GOX is used to react with glucose to form hydrogen peroxide which is subsequently measured at the working electrode. However due to unwanted background interferences, the first working electrode 922a will measure both the glucose signal as well as interference signals such as those produced by ascorbic acid and acetaminophen. The second working electrode 922b, which acts as a blanking electrode, may be coated with catalase which is a natural consumer of hydrogen peroxide and thus will prevent the second working electrode 922b from receiving the hydrogen peroxide that is created when glucose interacts or reacts with GOX. The removal of the peroxide will allow the second working electrode 922b to react to and only measure the background interferences. The electrical signal from the second working electrode 922b is subsequently subtracted from the electrical signal from the first working electrode 922a via an analog differential circuit or using digital electronics and software to yield the desired analyte signal.

A feature that may be provided in conjunction with essentially any electrochemical sensor design is to add a spare set of electrochemical electrodes or, in some embodiments, only spare working electrode(s), which would remain dormant until needed. The spare electrode(s) would remain disconnected (e.g., through high impedance) through internal electronics to prevent the attraction of fouling agents. Once a currently active electrode shows signs of diminished performance or diminished sensitivity, or after a predetermined period of time, a spare electrode (e.g., a spare working electrode) may be activated. The spare electrode may operate in conjunction with the originally activated electrode until it is “settled” and checked for accuracy and then would become the active electrode while the original electrode is disabled. The process may continue until all spare electrodes have been consumed.

### Electrode Compositions/Materials

Working electrodes for the various chemical cells described herein may be constructed with a reaction surface that is made from one or more of the following materials, among others: platinum, palladium, gold, rhodium, Prussian blue, Prussian blue analogs, carbon or carbon nanotubes. In addition, to pass the electrical signal from the surface material of the electrode to the system electronics, additional metals or materials may be employed that are of lower cost, such as carbon, silver, copper, combinations thereof, or any other acceptable electrical conductor.

The reference electrode used for the various 3-terminal and 3-terminal cells described herein, in conjunction with the material chosen for the working electrode, determines the oxidation or reduction voltage required to drive the cell for measuring a desired analyte. The reference electrode is commonly constructed from a mix of silver and silver chloride, typically in a respective ratio of 60/40 due to its electrochemical stability. Other ratios of silver and silver chloride may be employed as well as other materials. Other materials include platinum, palladium, gold, rhodium, Prussian blue, Prussian blue analogs, carbon or carbon nanotubes, graphene or graphene doped with metal alloys, among others. In addition, to pass the electrical signal from the surface material of the reference electrode to system electronics, additional metals or materials may be employed that are of lower cost such as carbon, silver, copper, combinations thereof, or any other acceptable electrical conductor.

The counter electrode for the various 3-terminal cells described herein serves to compensate for any impedances present in the cell. These impedances may be due to salts or other compounds that result in a voltage drop in the cell as the measured analyte current increases. The counter electrode voltage will change automatically to ensure that the excitation voltage in the cell between the reference and working electrodes is maintained. Alternatively, in the various 2-terminal cells described herein, the reference and counter terminals are commonly combined into a counter/reference terminal. The counter or counter/reference electrode is commonly constructed from a mix of silver and silver chloride, typically in a respective ratio of 60/40 due to its electrochemical stability. Other ratios of the silver and silver chloride may be employed as well as other materials. Other surface materials for the counter or counter/reference electrode include platinum, palladium, gold, rhodium, Prussian blue, Prussian blue analogs, carbon or carbon nanotubes, graphene, or graphene doped with metal alloys, among others. In addition, to pass the electrical signal from the surface material of the counter or counter/reference electrode to system electronics, additional metals or materials may be employed that are of lower cost such as carbon, silver, copper, combinations thereof, or any other acceptable electrical conductor.

### Electrochemical Process Configurations

Electrochemical cells for measuring glucose or other analytes may be constructed with electrodes of any of the configurations described above. Additionally, various chemical elements may be added to the cell to promote the collection and measurement of the desired analyte.

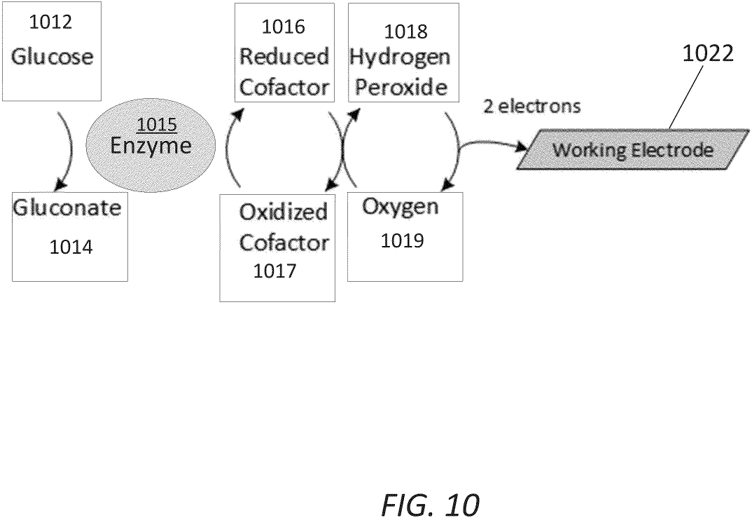

In various embodiments, and with reference to FIG. 10, glucose may be indirectly measured through generated hydrogen peroxide. In one possible construction method, glucose oxidase (GOX) enzyme 1015 is cast or dried on a surface of a working electrode 1022, for example, in a matrix of a hydrogel or polyethylene glycol surface group. Once activated through rehydration, the enzyme 1015, in the presence of glucose 1012 in the body, a cofactor in the body (which can be in a reduced cofactor 1016 state or an oxidized cofactor 1017 state), and oxygen 1019 from the air or the body, will form gluconate 1014 and hydrogen peroxide 1018. For measurement, the hydrogen peroxide will subsequently be either reduced or oxidized depending on the electrode and voltage configuration of the cell, and the reaction will produce a fixed number of electrons (i.e., 2) for each glucose molecule converted. The electrons will subsequently be measured as an electrical signal, producing a quantitative value that corresponds directly to the glucose concentration. The described method is only one method of measuring glucose. Other methods may also be used.

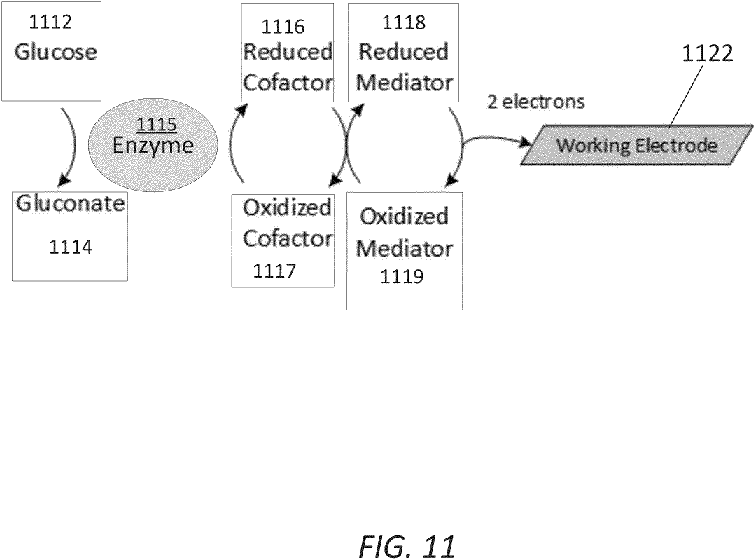

In various embodiments, and with reference to FIG. 11, glucose may be indirectly measured through a mediator. In one possible construction method, glucose oxidase enzyme 1115 is combined with a mediator (which may have a reduced mediator 1118 state or an oxidized mediator 1119 state) and cast or dried on a surface of a working electrode 1122, for example, in a matrix of a hydrogel or polyethylene glycol surface group. Once activated through rehydration in the body, the enzyme with a combined mediator and in the presence of glucose 1112 and cofactor (which may have a reduced cofactor 1116 state or an oxidized cofactor 1117 state) will convert the glucose 1112 to gluconate 1114 and electrons for measurement, as measured by an electrode, for example, electrode 1122. The mediated glucose oxidase is either reduced or oxidized depending on the electrode and voltage configuration and the reaction will produce a fixed number of electrons for each glucose molecule converted. The electrons will subsequently be measured as an electrical signal producing a quantitative value that corresponds directly to the glucose concentration. This method of measuring glucose does not require the need for oxygen, which may be limited in the body. Mediators that are commonly available and are suitable for this reaction include potassium ferricyanide, hexaammineruthenium(III) chloride, and methoxy phenazine methosulfate, among others.

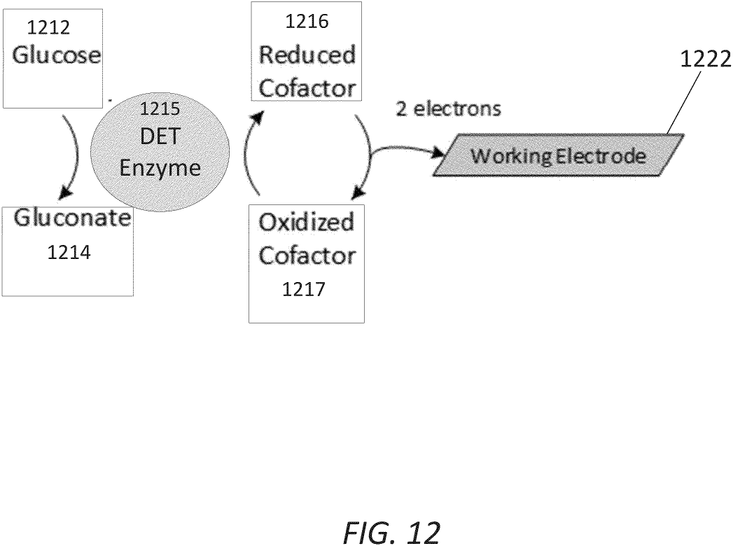

In various embodiments, and with reference to FIG. 12, glucose may be measured through a Direct Electron Transfer (DET) glucose reactive enzyme. In one possible construction method, DET glucose reactive enzyme 1215 is cast or dried on a surface of a working electrode 1222, for example, in a matrix of a hydrogel or polyethylene glycol surface group. The enzyme 1215, once activated through rehydration on the body and in the presence of glucose 1212 and cofactor (which has a reduced cofactor 1216 state and an oxidized cofactor 1217 state), will convert the glucose 1212 to gluconate 1214 and electrons for measurement. The enzyme is either reduced or oxidized depending on the electrode and voltage configuration and the reaction will produce a fixed number of electrons for each glucose molecule converted. The electrons will subsequently be measured as an electrical signal producing a quantitative value that corresponds directly to the glucose concentration. This method of glucose measurement has become recently available with a few DET enzymes currently available.

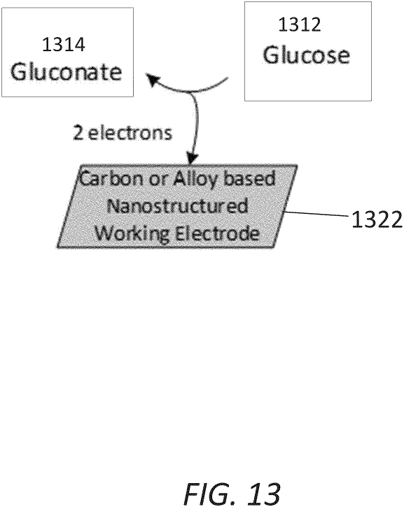

In various embodiments, and with reference to FIG. 13, glucose may be measured by direct electron transfer. One possible construction method is to use a working electrode 1322 that is comprised of carbon nanotubes, graphene or graphene doped with metal alloys and/or alloy nanostructures containing platinum, lead, gold, palladium, and/or rhodium. In the presence of glucose 1312 the working electrode material will convert glucose 1312 directly to gluconate 1314 and electrons for measurement when the correct excitation voltage is applied. This reaction is ideal as it does not use any enzyme or mediators to measure the glucose concentration. The electrons will subsequently be measured as an electrical signal producing a quantitative value that corresponds directly to the glucose concentration. This method of glucose measurement is emerging with the development of newer nanotechnologies and manufacturing systems and has proven to be a viable alternative to the more traditional glucose sensing methods.

Various configurations of electrochemical cells used as analyte sensors will now be discussed. The electrochemical cells for measuring glucose or other analytes may be constructed on a plastic or metal needle or cannula with electrodes that could be printed, plated, precast, embedded, or fabricated using any other method required to construct the electrode with the correct materials and electrical conduction pathways needed to send the signal back to the internal electronics for processing. In general, the electrochemical cells require a working electrode, a counter electrode, and a reference electrode. In some embodiments, the counter electrode and reference electrode may be combined into one electrode.

FIGS. 14(A-C) and FIGS. 15(A-C) illustrate various exemplary shapes, patterns, and orientations of the 2-electrode and 3-electrode embodiments of the electrochemical cell. It should be noted that these illustrations are exemplary in nature and are not meant to limit the scope of the invention. As may be realized by one of skill in the art, other configurations and arrangements of components and methods of manufacturing are possible and are contemplated to be within the scope of the invention.

For purposes of explanation, the illustrations of the electrochemical cells in FIGS. 14(A-C) and FIGS. 15(A-C) are shown as if the round cannula or needle was cut open over its length and pressed flat, with the exterior of the cannula or needle depicted. FIG. 14A shows a two-electrode sensor utilizing a working electrode 1402 and a combined counter/reference electrode 1404. FIG. 14B shows a three-electrode sensor having working electrode 1402 and wherein the counter and reference electrodes are shown separately and indicated as reference numbers 1406 and 1408 respectively. FIG. 14C also shows a three-electrode sensor having three working electrodes 1402 and separate counter and reference electrodes 1406 and 1408 respectively.

FIGS. 15(A-C) show various embodiments of sensors wherein the electrodes are configured as bands wrapped around the needle or cannula. FIG. 15A shows a two-electrode sensor having a working electrode 1502 and a combined counter/reference electrode 1504. It should be noted that the wire 1503 extending from the counter/reference electrode 1504 is isolated. Under the working electrode 1502. FIG. 15B shows a configuration having dual two-electrode sensors each having a working electrode 1502 and a combined counter/reference electrode 1504. This embodiment could be used, for example, to sense two separate analytes. FIG. 15C shows a three-electrode sensor having working electrode 1502 and reference electrode 1508 and wherein the counter electrode 1506 is configured as a wire mesh over the surface of the cannula.

The electrochemical cells require reactive chemical components to be placed over the electrode surfaces for the sensors to function properly. These chemical components are used to enable diffusion of an analyte from the body via bodily fluids or fluids pre-supplied during the manufacturing process to allow the analyte to react fully with the embedded chemical species and/or react directly with the electrodes in the chemical cell. For example, a hydrogel that may contain a sterilized component and water from the manufacturing process may be prepacked with the sensor to shorten the sensor startup time and increase the diffusion of the analyte into the electrochemical cell and produce chemicals out of the sensor. Alternatively, another example could be a sensor wherein a hydrogel is applied to the electrodes to enable the transport of glucose molecules directly to a carbon nanotube, graphene, or graphene doped with metal alloys electrode where the glucose molecule is directly converted into a quantifiable electrical signal.

The use of enzymes to convert glucose to either a second reactive component or directly to electrons requires a manufacturing technique to install and dry the enzyme onto the electrodes of the cell. In one embodiment, this could be achieved by directly mixing the enzyme with an electrode ink and drying the combination in a manner that allows the ink to cure and the enzyme to remain alive and active. Other methods of installing and drying the enzyme onto the electrode of the cell could also be used. Alternatively, various gels and polymers can be employed to cast and dry the enzyme with other chemicals such as surfactants and stabilizing compounds to ensure the enzyme is stable and able to receive the analyte and conduct the appropriate chemical constituents to the electrodes for reduction, oxidation, and measurement of the resulting electron transfer.

It is not uncommon for the working electrode in an electrochemical cell to become fouled or damaged by unwanted chemical species that collect on the surface of the electrode during normal operation. Some methods to protect the electrode(s) are to overlay the electrodes with a Nafion®, polysulfone, cellulose, or other blocking membrane material to keep fouling molecules away from the sensitive working electrode. Specifically, the coatings should have pore size selectivity for the hydrogen peroxide molecules only and reject large potentially fouling molecules such as phenolic resins that are commonly found in insulin.

In some embodiments, the electrode(s) may be sterilized before use. Accordingly, the materials used to form the electrode and/or to overlay the electrode may be selected based on their tolerance of the sterilization process. Furthermore, excess enzymes or a sacrificial coating may be employed to protect the sensor(s) throughout the sterilization process. For example, excess enzymes may be coated onto the electrode(s), and the type of enzymes and thickness of the coating may be varied based on an expected amount of enzyme that will be removed from the electrode(s) during sterilization. Sacrificial coatings may involve protective coatings, surface finishes, or sacrificial protective layering that is configured based on an expected amount of the sacrificial coating that will be removed during the sterilization process. In exemplary embodiments, the sterilization process may be associated with parameters (such as temperature, pressure, etc.) that describe how sterilization occurs. The materials that form or overlay the electrode, the enzyme materials and thicknesses, and properties of the sacrificial coatings may be selected based on the sterilization process parameters.

An added benefit to a membrane is that it can act as a structure for holding the necessary chemical components of the electrochemical cell in place over the electrodes. Enzymes and gels can be cured onto the structure of the membrane as part of the cell construction process.

Additionally, the whole sensor can be overcoated with an additional membrane to limit the influx of glucose. Careful selection of the pore size and quantity of pores is important to allow glucose molecules through while limiting their quantity. Pore sizes must be greater than the size of the glucose molecule (e.g., > ~1 nm). The hole sizing can be determined through research and experimentation so that it is large enough to allow glucose molecules to pass through without being so large that unwanted molecules pass through as well. The quantity of holes helps to control the rate of conversion from glucose to hydrogen peroxide because the reaction requires oxygen which may not be as abundant as glucose when the sensor is operating inside the body. The lack of oxygen will therefore limit the conversion of glucose to hydrogen peroxide which will directly affect the glucose value computed by the electronics and software system.

In one embodiment described above with respect to FIG. 15B, a single sensing device or needle may be provided with multiple electrochemical sensing regions. The additional sensing regions would each require their own electronic analog front end to provide and maintain the proper excitation voltages. The goal of the additional sensing elements is to tune the additional sensing regions for specific sensing characteristics by having individualized sensor electrode sizes, materials, coatings, enzymes, chemical enhancers, and membranes. There are many possible combinations and configurations that may be used to provide a complete sensing solution where the one sensor may have limited range but be capable of rapid response to rising and falling glucose values. This is achieved by making the sensor smaller, with thinner hydrogel and filtering components allowing for fast ingress and diffusion of the glucose molecules to the sensing region. Because a sensor like this may be easily overwhelmed by high glucose levels or fouling agents, its positioning away from the insulin delivery source and possibly high in the skin may provide natural protections from fouling of the sensor electrodes. Additional sensing regions could be employed with larger electrodes, thicker gels, coatings and membranes with slower but more averaged response and greater overall measurement range.

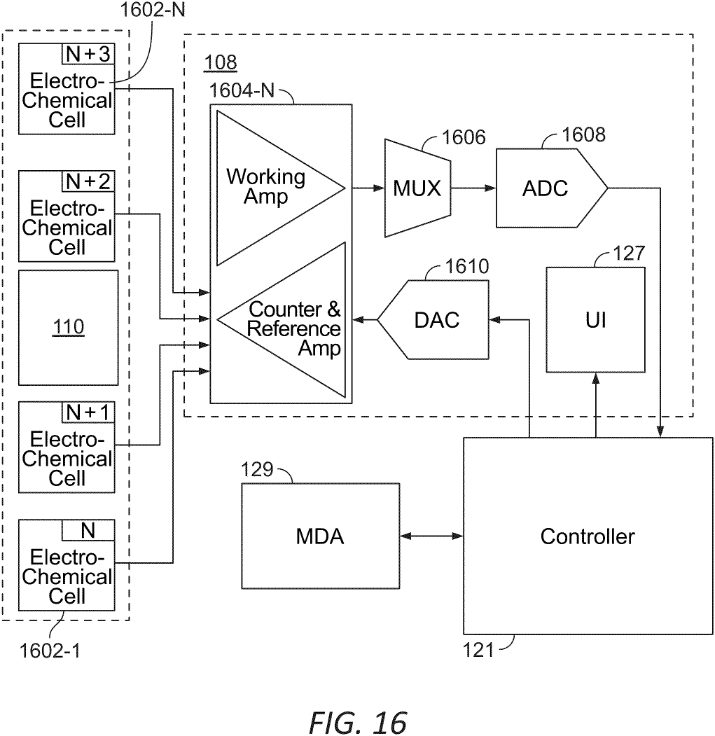

FIG. 16 is a block diagram of a possible implementation of the electronics and control system to drive the sensors and collect the quantifiable electrical signal data from the various electrochemical cell implementations. In one embodiment, the electronics and control systems may be SPAID 102 electronics, and the control system may be implemented separately. The interpretation of the data collected from the sensors will be performed by the software algorithm and must be tailored for each individual sensor taking into account the response characteristics of each sensor and the resulting digitized analog signal. In some embodiments, the software algorithm may be implemented as part of MDA 129 or user app 160 or may be implemented as separate software stored in memory 123 and executed by controller 121.

With reference to FIG. 1 and FIG. 16, sensor patient interface 110 may comprise one or more electrochemical cells 1602-1 ... 1602-N. In some embodiments each of electrochemical cells 1602-1 ... 1602-N may be configured to detect glucose while, in other embodiments, some of electrochemical cells 1602-1 ... 1602-N may be configured to detect glucose and others may be configured to detect other analytes. Raw signals from electrochemical cells 1602-1 ... 1602-N in sensor patient interface 110 are sent to analyte sensor 108. Within analyte sensor 108, the signals are amplified at 1604, multiplexed at 1606 and converted to a digital signal by analog-to-digital converter 1608. Amplifiers 1604 are preferably suitable for maintaining a proper excitation voltage for each electrochemical cell throughout its use cycle. Once converted to a digital signal, the software algorithm, which may be implemented as part of MDA 129 executing on controller 121 will analyze the raw signals of glucose readings or readings of levels of other analytes. MDA 129 may determine, from the resulting glucose readings, if an alarm or alert is necessary and, if so, may send a user feedback through user interface 127. MDA 129 and converted from digital signals to analog signals by digital-to-analog converter 1610 and amplified by amplifier 1604 prior to being used by electrochemical cells 1602-1 ... 1602-N. It should be noted that electrochemical cells 1602-1 ... 1602-N receive power from amplifiers 1604-1 ... 1604-N to keep the electrochemical cells active.

### Opto-Fluorescent Sensing Methods

Opto-fluorescent sensing can be used to sense glucose in the various drug delivery systems described herein, including the various needle or cannula-based sensing systems described herein. Sensing may be performed by single or multiple opto-fluorescent sensing regions on or within a given needle or cannula to enhance reliability, accuracy, endurance and overall performance. Alternatively, an opto-fluorescent sensing element may be separately deployed at the time of device installation and removed at the end of the device lifecycle, if desired.

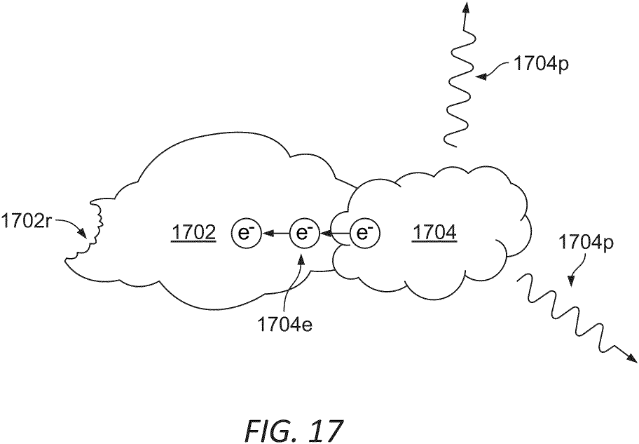

One common method of opto-fluorescent sensing is based on the Förster (or Fluorescence) Resonance Energy Transfer or FRET method. With reference to FIGS. 17-20, in the method shown, which illustrates a direct glucose to light method, a receptor fluorophore (commonly a fluorescent dye) is bound to an acceptor molecule. The acceptor (also known as a quencher) is capable of binding to both glucose and to the receptor fluorophore. With specific reference to FIG. 17, the acceptor molecule 1702 will pull electrons 1704e from the receptor fluorophore 1704 when it is not also reversibly bound to glucose (a glucose receptor with no glucose is designated by 1702r). This pulling of electrons is known as FRET quenching. FRET can be thought of as a process whereby the acceptor molecule 1702 (quencher) suppresses fluorescence from the receptor fluorophore 1704 (e.g., a fluorophore dye) by draining energy from it. This limits the receptor’s ability to emit energy as photons 1704p once it has received input excitation photons. The energy drained away from the receptor fluorophore 1704 is in the form of electrons 1704e. This occurs when the physical distance between the acceptor 1702 and the receptor 1704 is small allowing for the close transfer of electrons which by definition is FRET quenching. Without glucose, the quenching process is strong and the acceptor 1702 is able to draw electron energy from the light-emitting Fluorophore 1704 thus reducing its light output 1704p.

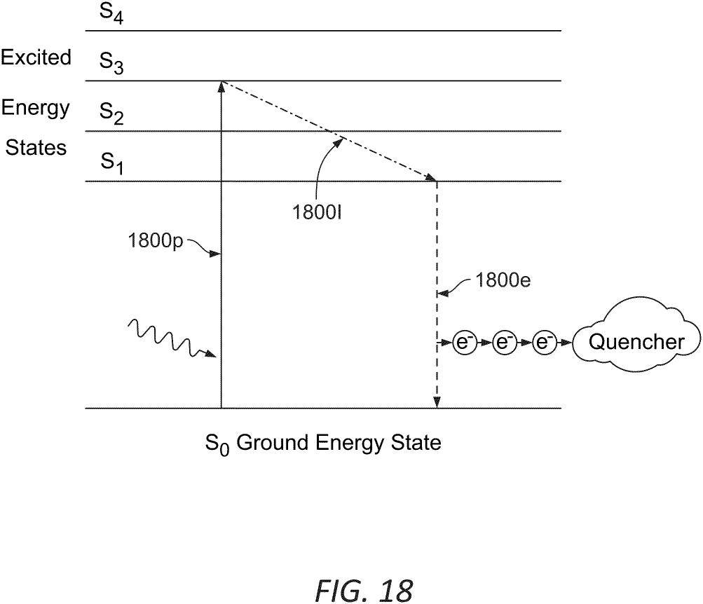

FIG. 18 shows the energy excitation 1800p, energy loss 1800l and energy emission 1800e states of the fluorophore when there is FRET quenching. The absorbed excitation light from an external source is received by the fluorophore and elevates it from the ground energy state S₀ to a higher energy state S₃. Once elevated, fluorophore energy is quickly dissipated in the form of either heat or vibrational energy 1800l and the energy level drops to the lowest level of the excited energy states S₁. This loss of energy happens in just a few nanoseconds and is referred to as Excited Energy Lifetime. Lastly when FRET quenching is active the fluorophore releases its energy as electrons to the quencher rather than as photons. Once most of the energy in the form of electrons is transferred to the quencher the fluorophore is returned back to the ground energy state S₀ to begin the cycle again if excited.

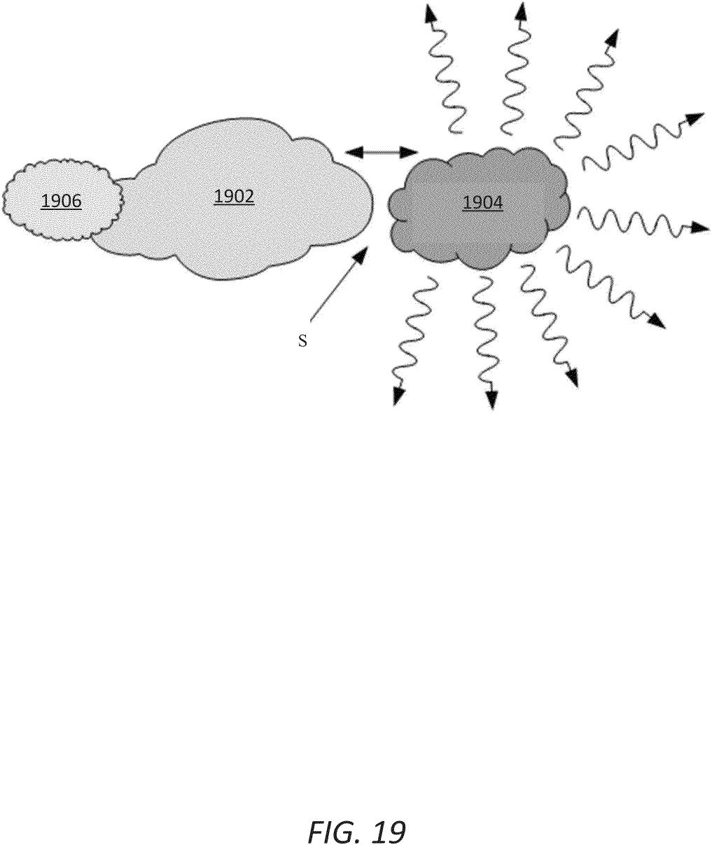

With reference now to FIG. 19, when glucose 1906 binds to the fluorescence acceptor (quencher) 1902, a structural change occurs that moves the fluorescence donor (fluorophore) 1904 and fluorescence acceptor (quencher) 1902 farther apart. This subsequently decreases electron transfer between the fluorophore 1904 and acceptor 1902, which reduces FRET quenching and thus frees the fluorophore 1904 to radiate more energy as light rather than having it quenched through an electron energy transfer to the acceptor 1902. Thus, an increased glucose 1906 concentration is associated with more radiated light from the fluorophore 1904 once it has received excitation light energy from an external source. The glucose binding to the receptor is a function of glucose concentration in the solution that this acceptor/receptor combination is operating in. The glucose binding is reversible and will decrease as the analyte glucose concentration decreases.

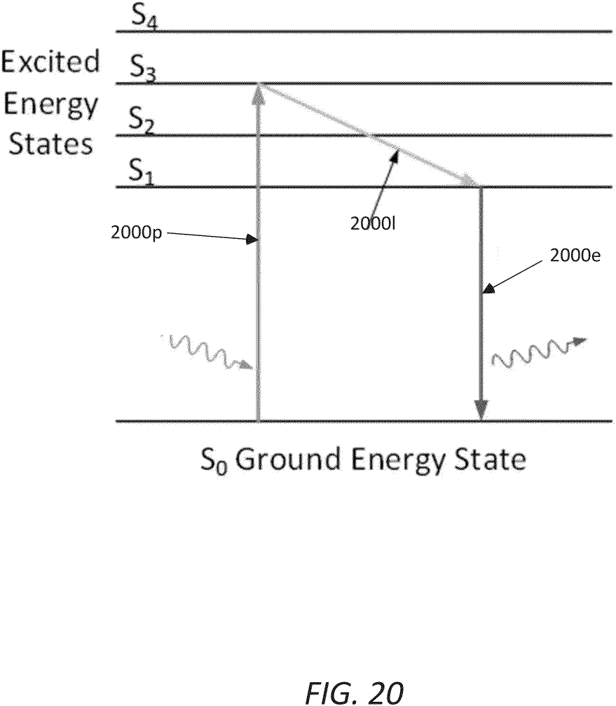

FIG. 20 shows the energy excitation 2000p, energy loss 2000l and energy emission 2000e states of the fluorophore when there is no FRET quenching. The absorbed excitation light from the external source is received by the fluorophore and elevates it from the S₀ state to a higher energy state S₃. Once elevated, energy is quickly dissipated in the form of either heat or vibrational energy and the energy level drops to a lower-level excited states s₁. As noted above, this loss of energy happens in just a few nanoseconds and is referred to as Excited Energy Lifetime. Lastly, when FRET quenching is not active, the fluorophore releases its energy as a photon emission at a lower energy state S₁ than the initial input excitation photon energy state S₃ due to the loss during the Energy Lifetime transition. This release of energy shifts the color of the light emitted from the light initially absorbed to light of longer wavelength/lower energy. This results in a color shift from the violet end of the light spectrum towards the lower energy red end of the light spectrum.

Opto-fluorescent sensing can be used to sense glucose in any needle or cannula-based sensing systems. In addition, sensing can be performed by single or multiple opto-fluorescent sensing regions on a given needle or cannula to enhance reliability, accuracy, endurance and overall performance.

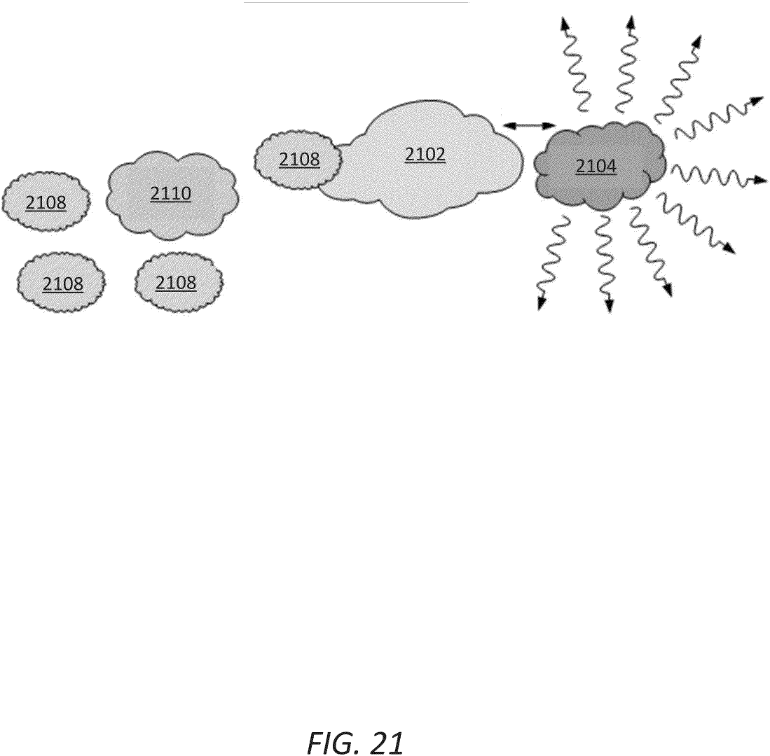

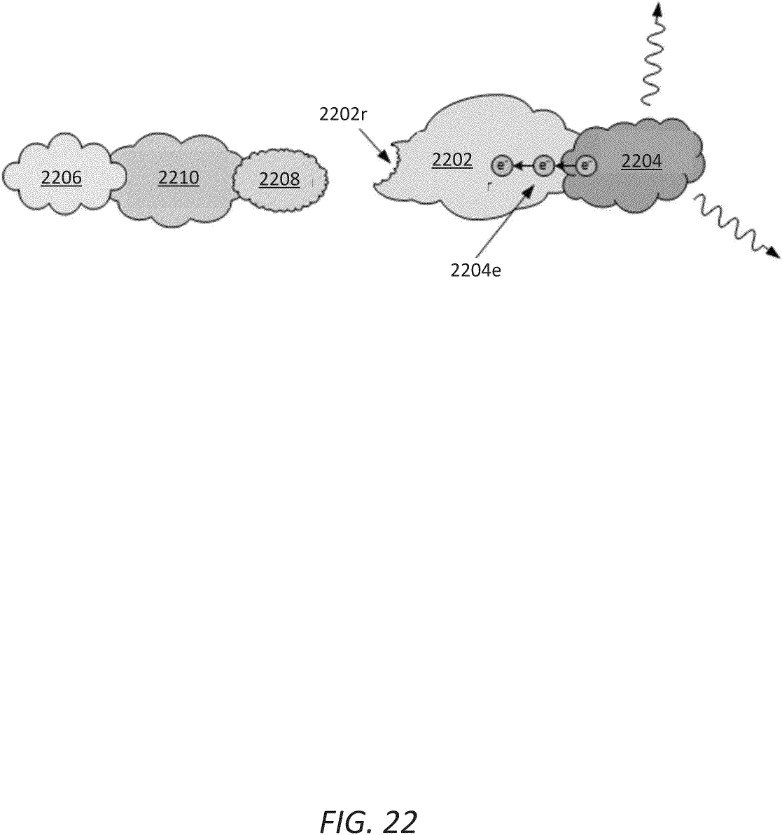

FIGS. 21 and 22 illustrate an indirect glucose to light method, wherein the acceptor is capable of binding to both oxygen and to the receptor fluorophore. With specific reference to FIG. 22, the acceptor 2202 will pull electrons (e⁻) from the receptor fluorophore 2204 when it is not also reversibly bound to oxygen (an oxygen receptor with no oxygen is designated by 2202r). This pulling of electrons is known as FRET quenching as explained previously. However, in this indirect glucose to light method, the glucose concentration in an analyte solution can be determined based the amount of oxygen 2108 available in the solution. The concentration of oxygen 2208 is reduced when glucose 2206 reacts with glucose oxidase 2210 to produce hydrogen peroxide. This reduction in oxygen due to an increased glucose concentration allows for an increase in FRET quenching and thus less proportional light output from the fluorophore as shown in FIG. 22. The opposite scenario is true where less glucose results in more oxygen 2108, which binds to the acceptor 2102, which moves the receptor fluorophore 2104 and acceptor 2102 farther apart (see FIG. 2). This subsequently decreases electron transfer between the receptor fluorophore 2104 and acceptor 2102 which reduces FRET quenching and thus frees the receptor fluorophore 2104 to radiate more energy as light rather than having it quenched through an electron energy transfer to the acceptor 2102. Thus, a decreased glucose concentration is associated with more radiated light from the receptor fluorophore 2104 once it has received excitation light energy from an external source. The oxygen binding is reversible and will decrease as the analyte glucose concentration increases.

In all embodiments using the opto-fluorescent sensing method, the sensing material produces a quantifiable light signal where the magnitude and duration are quantifiable and in proportion to the glucose concentration once it has been excited by the light source and this process works both with increasing and decreasing concentrations. In addition, the length of time of the fluorophore light emission after being excited by a light source is also proportional to the glucose concentration. Both quantifiable measurement methods could be employed to calculate the glucose concentration by MDA 129 or user app 160.

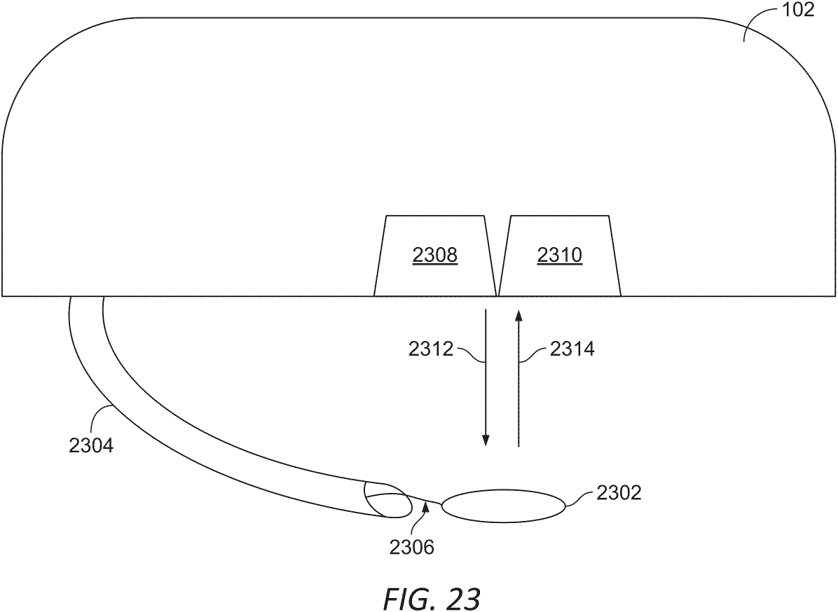

FIG. 23 shows one possible glucose sensor configuration for the direct glucose-to-light method of opto-fluorescent sensing that uses a deployable opto-fluorescent sensing element 2302. In one embodiment, sensing element 2302 is deployed through cannula 2304 or otherwise pushed into the skin with a retractable needle or an alternative mechanism. Sensing element 2302 is designed to fluoresce in the presence of glucose and should be implanted in the subcutaneous (sub-Q) region of the skin directly below the base of SPAID 102.

If sensing element 2302 is installed using cannula 2304, the cannula installation mechanism would push the element into the skin during the initial insertion and then cannula 2304 could retract slightly to leave the element in the subcutaneous area of the skin just beyond the tip of cannula 2304. Sensing element 2302 would remain mechanically connected to cannula 2304 via a tether, cord, or cable 2306. At the end of the system lifecycle, cannula 2304 and sensing element 2302 would be retracted together with tether 2306 being used to retrieve sensing element 2302.

Once implanted the opto-fluorescent sensing element 2302 will be used to produce a measurable optical signal in proportion to glucose. An excitation light source 2308 designed to produce light at the correct wavelength necessary to excite the fluorophore on the sensing element is incorporated into the base of the housing of SPAID 102. Preferably, the deployment mechanism of SPAID 102 will place sensing element 2302 directly below excitation light source 2308 at a depth of ~2-5 mm such that light emitted from excitation light source 2308 follows path 2312 to sensing element 2302. SPAID 102 also incorporates a light detector 2310 to be mounted in the base of the housing of SPAID 2310 directly above sensing element 2302. In some embodiments, excitation light source 2308 and light detector 2301 may be integrated into a single unit. Light detector 2310 may have selective filtering to allow it to detect emission light having the wavelength that is produced by the fluorophore in the sensing element. The emission light follows path 2314 to light detector 2310. Due to the FRET effect, the excitation light and the emission light are of different frequencies which helps to reduce “cross-talk” between the excitation light and the emission light.

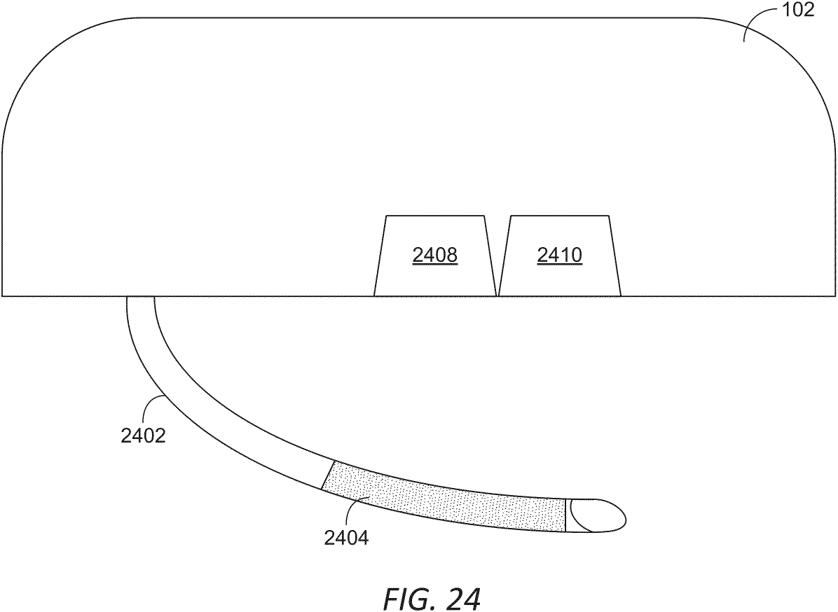

FIG. 24 shows an alternate glucose sensor configuration using the direct glucose to light method of opto-fluorescent sensing. In this embodiment, an opto-fluorescent sensor coating 2404 is disposed on the surface of a cannula 2402 or needle. The cannula 2402 or needle with the opto-fluorescent sensing region 2404 is implanted using a mechanism that allows the sensing region 2404 to be positioned directly (or nearly directly) below an excitation light source 2408 and detector 2410 that are mounted in the base of the housing of SPAID 102.

Once implanted, the opto-fluorescent sensing region 2404 on the cannula 2402 or needle produces a measurable optical signal in proportion to glucose when excited by light of a specific wavelength. An excitation light source 2408 designed to produce light at the correct wavelength necessary to excite the fluorophore on the sensor is included in SPAID 102 and is preferably incorporated into the base or bottom portion of SPAID 102. The deployment mechanism of SPAID 102 is designed to place the sensing region 2404 directly below the excitation light source 2408 at a depth of ~2-5 mm. SPAID 102 also incorporates a light detector 2410 also mounted in the base or bottom portion of SPAID 102 directly above sensing region 2404. In some embodiments excitation light source 2408 and light detector 2410 may be integrated into a single unit. Light detector 2410 has selective filtering to allow it to detect only light of the wavelength that is produced by the fluorophore in sensor 2404. Due to the FRET effect, the excitation light 2408 and the light emitted by sensor 2404 are of different frequencies which reduces cross talk between the sourced and detected light.

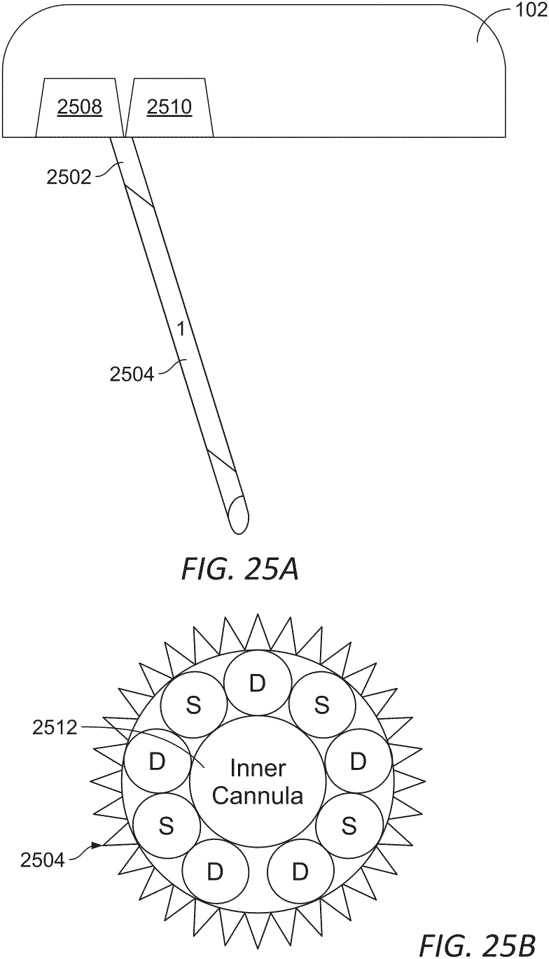

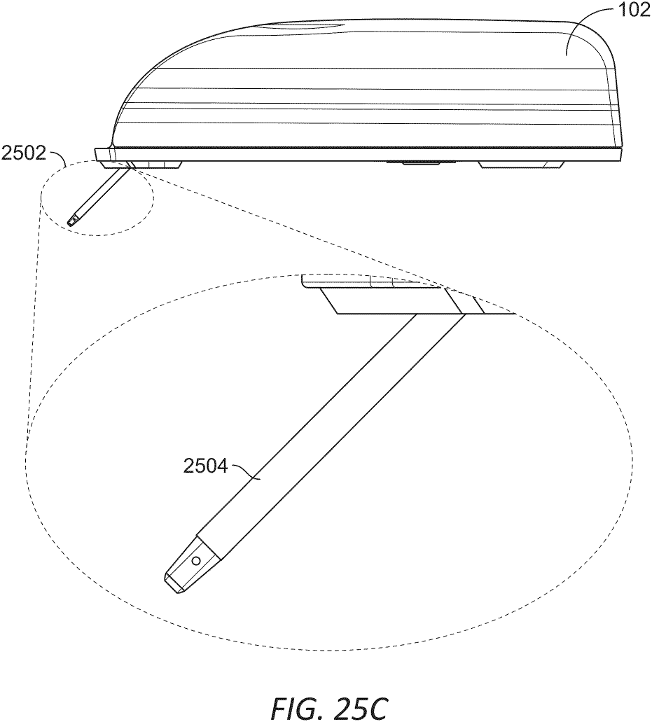

FIG. 25A shows another possible embodiment of a glucose sensor configuration using the direct glucose to light method of opto-fluorescent sensing which uses an opto-fluorescent sensor coating 2504 mounted on the surface of a cannula 2502 or needle. The cannula 2502 or needle with the opto-fluorescent sensing region 2504 is implanted in the subcutaneous region of the skin below the base of the housing of SPAID 102. In alternative embodiments, the opto-fluorescent sensing region may be provided on a separate needle, as shown in FIG. 5A.

Once implanted, the opto-fluorescent sensing region 2504 on the cannula 2502 or needle produces a measurable optical signal in proportion to glucose. An excitation light source 2508 designed to produce light at the correct wavelength necessary to excite the fluorophore on the sensor 2504 is incorporated inside the housing of SPAID 102. The cannula 2502 or needle is constructed using light pipe or fiberoptic light-conducting materials, as shown in FIG. 25B, that may be used to conduct the excitation light as well as deliver the light emitted by sensor 2504 to and from SPAID 102 to the opto-fluorescent sensing region 2504. Alternatively, the cannula 2502 or needle could be constructed of alternative materials with embedded light conductors in the walls of the device.

FIG. 25B depicts the light conducting elements (“S” and “D”) embedded in the wall of cannula 2502 or a needle. The quantity, ratio and location of the light conductors may vary. The light sourcing conductors (“S”) are alternated with the light detecting conductors (“D”) to improve illumination for the fluorophore excitation and the detection from the fluorophore emission.

The light source 2508 and detector 2510 are mounted inside or are part of SPAID 102 and are coupled to the light conductors. The light source 2508 and detector 2510 may comprise a combination device where both devices are in a single package, allowing for both to share the aperture of the light conductors. Alternatively, light could be coupled into the light conductor aperture using partial mirror devices (not shown) or other light directing devices (not shown) to allow the light source 2508 and light detector 2510 to be mounted separately from each other. In an alternative embodiment, multiple light conductors, as shown in FIG. 25B as “S” and “D” could be employed to conduct light to and from the opto-fluorescent region on the cannula or needle. In this case, a portion of the light conductors could be dedicated to sourcing light (“S”) while the others (“D”) are used for conducting the detected light back to light detector 2510. This would allow for separate light sourcing and detecting components to be used and mounted separately inside of the housing of SPAID 102. Inner lumen or inner cannula 2512 may be used to deliver the liquid drug.

FIG. 25C shows a concept image of this embodiment, showing an enlarged view of the cannula 2502 in the inset.

In this embodiment, it is necessary to allow the excitation light to be diverted in an orthogonal direction to allow it to interact with the fluorophore material in sensing region 2504 mounted on the surface of the cannula 2502 or needle. In this embodiment, the excitation light illuminates the fluorophore material from the backside of the sensing region 2504 and the emitted light will additionally be produced and travel in the reverse direction back to SPAID 102. During the manufacturing process, the light pipe walls are “fractured” in a controlled manner to allow light to leak out of the sidewall of cannula 2502 to interact with the fluorophore material in sensing region 2504. Other methods of mirrors or surface scratching could also be used to allow light to exit and return to the surface of cannula 2502.

The light detector 2510 is configured with selective filtering to allow it to detect only light of the wavelength that is emitted by the fluorophore material in sensing region 2504. Due to the FRET effect, the excitation light and the emission light are of different frequencies, which reduces cross-talk between the sourced and detected light.

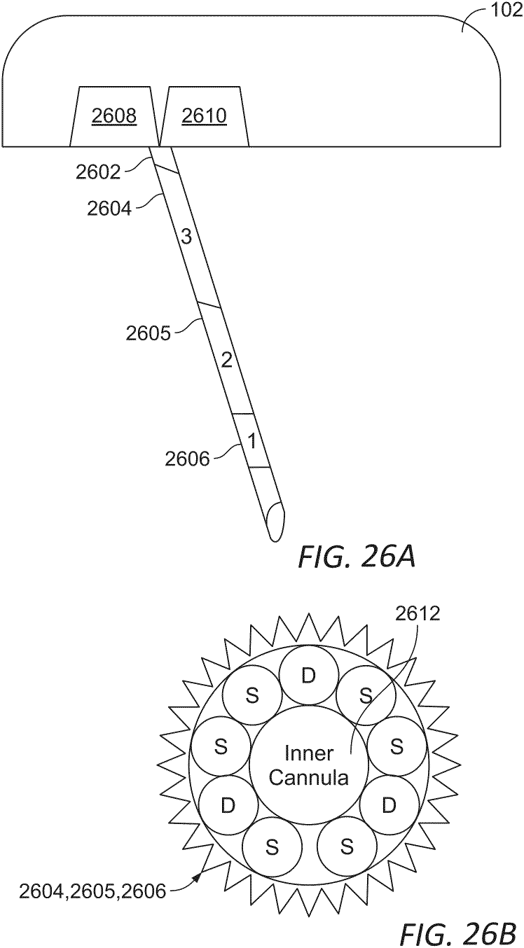

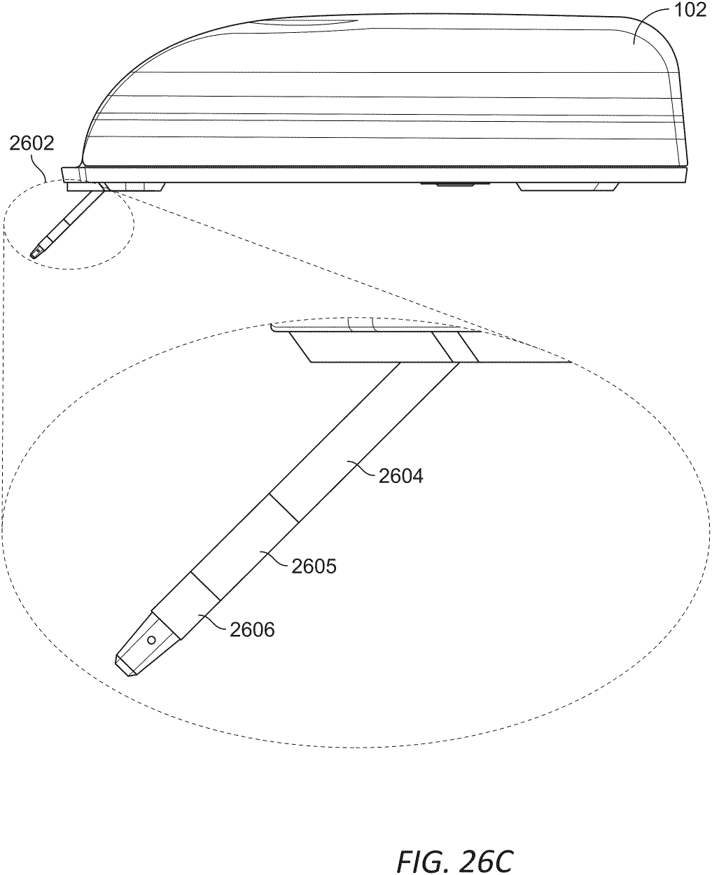

FIG. 26A shows yet another embodiment of an opto-fluorescent sensor mounted on the surface of a cannula 2602 or needle that is designed to fluoresce in the presence of oxygen. The sensor is used to produce a measurable optical signal in proportion to glucose by using glucose and glucose oxidase to reduce the local concentration of oxygen through the chemical reaction. The sensor produces a quantifiable light signal where the magnitude of the signal is in proportion to the oxygen concentration, which is directly proportional to the glucose concentration. Alternatively, it may produce a time-dependent glucose signal wherein the length of time the sensor produces a light signal after being excited by light source 2608 is proportionate to the glucose concentration. Either or both methods could be employed to calculate the glucose concentration. Light from light source 2608 is sent from SPAID 102 and sent down the cannula 2602 to the sensor region for exciting the opto-fluorescent sensor to produce return light. The light produced by the opto-fluorescent sensor travels directly through the walls of cannula 2602 or needle which comprises a light-conducting material similar to a light pipe or fiber optic. The light is then received inside of the housing of SPAID 102 by light detector 2610. In alternative embodiments, the opto-fluorescent sensing region may be provided on a needle or cannula separate from the cannula used to deliver the liquid drug, as shown in FIG. 5A.

The sensor can consist of three sensing regions 2604, 2605, 2606 of differing sizes to produce light signals that are proportional to differing concentrations of glucose, from low (2604) to medium (2605) to high (2606) concentrations, to enable more resolution in the measurement. A different number of sensing regions can be used (e.g., 2, 4, or more). Each of zones 2604, 2605, 2606 will have an individual light pipe(s), as shown in FIG. 26B, with light pipes conducting the excitation light labeled “S” and light pipes conducting the light emitted by sensing regions 2604, 2605, 2606 labelled “D”. Inner cannula 2612 may be used to deliver the liquid drug. As with the embodiment shown in FIG. 25B, the quantity, ratio, and location of the light conductors may vary. The light sourcing conductors (“S”) may be alternated with the light detecting conductors (“D”). FIG. 26C shows a concept image of this embodiment, showing an enlarged view of the cannula 2602 in the inset.

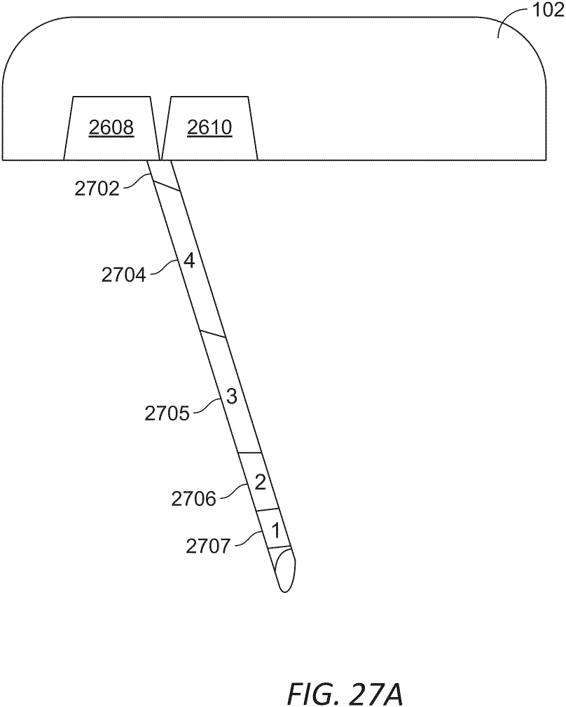

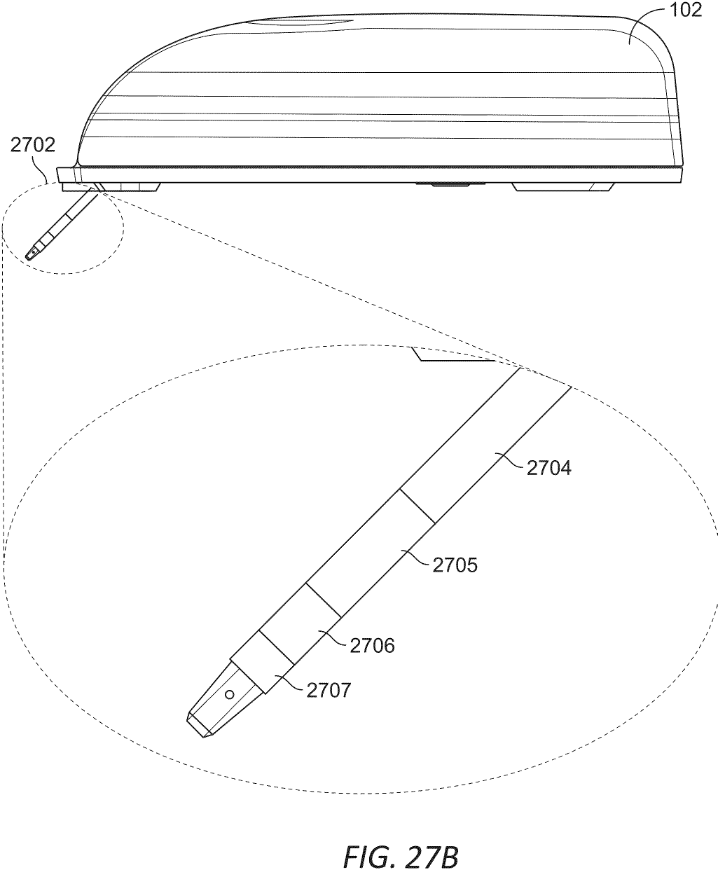

FIG. 27A is a schematic representation of a variation of the embodiment shown in FIGS. 26(A-C). As with the previous embodiment, the sensor can comprise three regions 2704, 2705, 2706 defined on cannula 2702 of differing sizes to produce light signals that are proportional to differing concentrations of glucose from low to medium to high. In addition, a fourth zone 2707 measures oxygen directly to establish a reference oxygen level in the sub-Q ISF (Interstitial Fluid). Each zone can have one or more individual light pipes with conducting excitation light (“S”) and emitted light (“D”). FIG. 27B shows a concept image of this embodiment, showing an enlarged view of the cannula 2702 in the inset. As with the previous embodiment, the opto-fluorescent sensing region may be provided on a needle or cannula separate from the cannula used to deliver the liquid drug, as shown in FIG. 5A.

### Opto-Fluorescent Materials

The direct and indirect glucose to light methods described above require various chemicals to construct a suitable glucose sensor. To implement the direct glucose to light conversion an acceptor/quencher molecule is needed that can both directly bind to the glucose molecule and to an opto-fluorescent dye. A commonly used molecule for this is Boronic Acid which when combined with a fluorescent dye can perform FRET quenching of the dye when the glucose molecule is reversibly bound. Once glucose is removed the FRET effect is extinguished and normal fluorescence emission is returned to the fluorophore.

In addition, to complete the implementation of the direct glucose to light conversion method, an opto-fluorescent dye is required that can bind with the acceptor/quencher. Ideally, the dye has a fairly significant shift from the wavelength of the excitation light to the wavelength of the emission light. This shift will simplify the construction of the source and detector system and reduce the possibility of cross-talk where emitted light is reflected and inadvertently detected. The dye will also simplify the implementation of wavelength-specific filters. The following are just a few commercially available dyes that would be suitable for this type of sensing method. Many alternatives are available and may be implemented. In addition, the system may also be constructed with multiple combined quencher and dye combinations to enhance selectivity, accuracy and broaden the working range of the sensor.

Commercially available dyes include the following, among others: (a) Becton Dickinson Pacific Blue™ (Ex-Max 401 nm/Em-Max 452 nm), which is based on the 6,8-difluoro-7-hydroxycoumarin fluorophore, and is strongly fluorescent, even at neutral pH, (b) Becton Dickinson Alexa Fluor® 488 (Ex-Max 495 nm/Em-Max 519 nm), whose conjugates are highly photostable and remain fluorescent over a broad pH range, (c) Becton Dickinson Horizon Brilliant™ Violet 605 (BV605) (Ex-Max 407 nm/Em-Max 605 nm), which is a tandem fluorochrome that combines BD Horizon BV421 and an acceptor dye with emission at 605 nm, and Becton Dickinson Horizon Brilliant™ Violet 650 (BV650) (Ex-Max 407 nm/Em-Max 650 nm), which is a tandem fluorochrome of BD Horizon BV421 and an acceptor dye with an emission maximum at 650 nm. For indirect glucose to light conversion, an acceptor/quencher molecule may be used to bind both molecular oxygen and an opto-fluorescent dye.

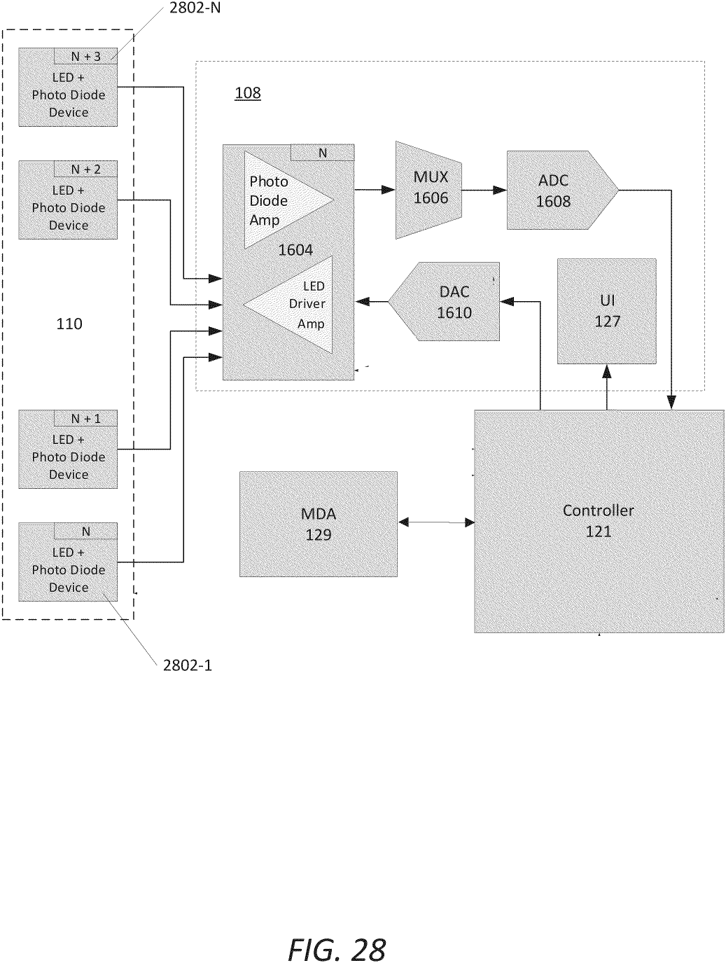

FIG. 28 is a block diagram of a possible implementation of the electronics and control system that may be used to drive the sensors and collect the quantifiable electrical signal data from the various opto-fluorescent sensing methods. The system is similar to the system shown in FIG. 16 with respect to electrochemical methods, except that, in this embodiment, sensor patient interface 110 comprises one or more opto-fluorescent sensing cells 2802-1 ... 2802-N, each cell comprising a light source, which, in a preferred embodiment, may be an LED or laser diode of a specific wavelength for emitting the excitation light, and a photodetection device for detecting light emitted by the fluorescent material of which the sensors are comprised. Otherwise, the raw signals from the opto-fluorescent sensing cells 2802-1 ... 2802-N are processed in the same manner as the electrochemical cells 1602-1 ... 1602-N, and the results used in the same manner as previously described.

### Sensing Methods Using Implanted Devices

Various sensing methods will now be described that use implanted sensors. In addition, various methods of delivering the liquid drug to the user will also be discussed.

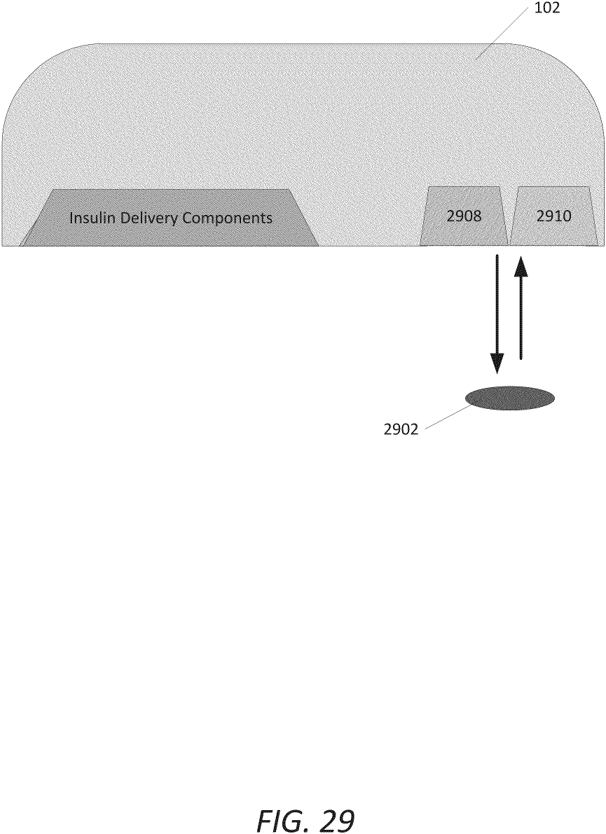

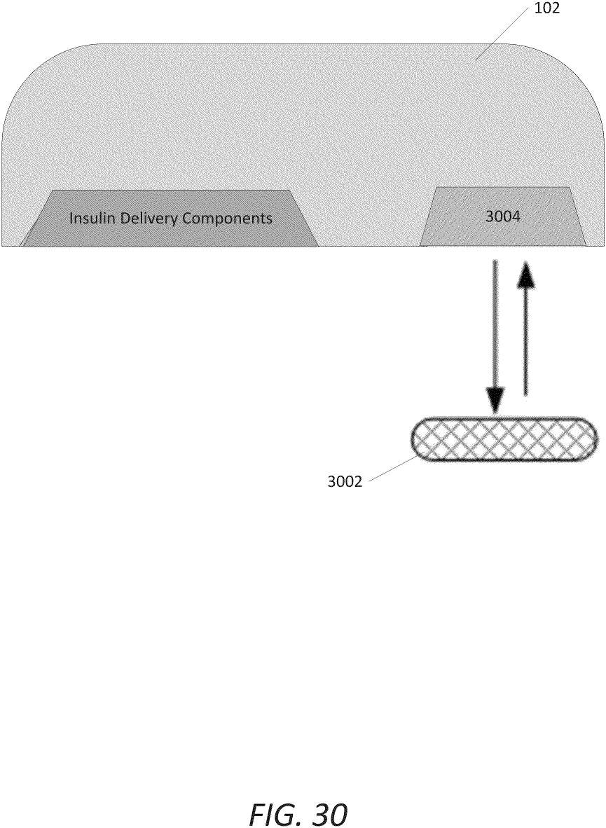

FIG. 29 shows an embodiment in which the glucose sensing is performed by mounting SPAID 102 directly over an implanted opto-fluorescent seed 2902. This embodiment operates in a similar manner to the opto-fluorescent methods previously described herein, except that there is no physical attachment between SPAID 102 and the opto-fluorescent seed 2902. In this embodiment, light is transmitted through the skin of the user by excitation light source 2908, and the light emitted by opto-fluorescent seed 2902 is detected through the skin by light detector 2910. This embodiment stimulates the implanted seed to make a glucose measurement and then processes the glucose value internally with MDA 129 or user app 160, which will use the values to deliver the required dose of the liquid drug insulin using one of multiple insulin delivery techniques described later herein. This embodiment may be used with the system shown in FIG. 28. FIG. 30 shows an embodiment in which the glucose sensing is performed by mounting SPAID 102 in close proximity to an integrated implanted glucose sensor 3002 configured to communicate with SPAID 102 via wireless communications 3004 to obtain the sensed glucose data. The implanted sensor 3002 contains a power source, a sensing system (opto-fluorescent, electrochemical, spectroscopy), and the necessary electronics to support the data processing, storage, and wireless communications with the body-worn transmitter or possibly direct to a smart mobile device. Wireless communications module 3004 in SPAID 102 may communicate with implanted sensor 3002 via one or more of several different methods, including, but not limited to Bluetooth (BLE) and / or Near Field Communications (NFC).

### Sensing Methods Using Microneedles

Glucose sensing can be performed in a SPAID 102 as described herein by using microneedle arrays mounted in the base of the device apart from the insulin delivery source. In some embodiments, for example, the SPAID 102 may comprise (a) a microneedle glucose sensing array combined with (b) an inserted insulin delivery cannula, a microneedle-based insulin delivery system, or a transdermal insulin delivery system. Each of these options with details around each implementation are explained below.

Electrochemical sensing can be used to sense glucose via a microneedle-based sensing system. In this embodiment, the sensing may be performed by microneedles that are coated with conductive electrochemical components allowing for the formation of an electrochemical cell circuit once the microneedles are inserted into the skin and interstitial fluid (ISF). The needles may be coated in any number of standard electrochemical conducting materials as described below.

The electrodes used to form the electrochemical cell(s) of the microneedle-based sensing system can be configured in multiple ways. The electrodes may be configured as individual needles or in clusters of needles to form the desired electrochemical sensing cells. The cells may be comprised of 2-electrode, 3-electode, 4-electrode or other multi-electrode combinations, with common or isolated electrical circuits being employed to create any number of sensing areas.

Chemically active materials, including enzymes, that may be required to construct the cell(s) can be coated directly onto electrodes on the surface of the microneedles, or may be embedded directly into the electrode materials themselves and deposited during the construction of the microneedle array. Various methods can be employed to apply the conductive materials and the chemically active materials such as vapor deposition, electroplating, spraying, printing, or using microelectronic fabrication methods.

Furthermore, the use of various fluidic conductive chemical components such as surfactants may be added to the surface of the needles to help increase the flow of bodily fluids (e.g., interstitial fluid) that are required to convey the glucose or other analytes to the surface of the microneedles.

Working electrodes (WE), reference electrodes (RE), counter electrodes (CE), blocking membranes and glucose-limiting membranes may be provided as discussed above.

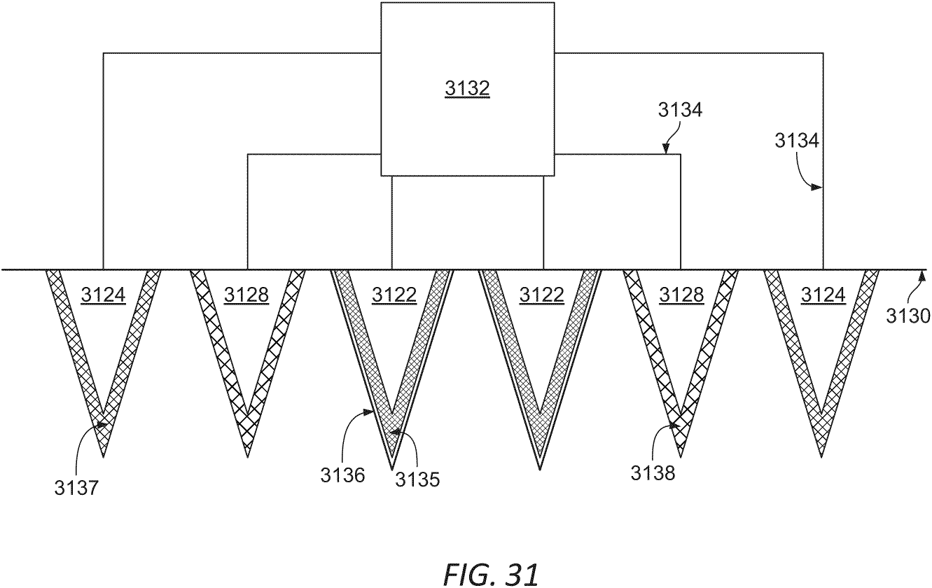

An embodiment of a microneedle-based sensing system containing two 3-electrode electrochemical cells is illustrated in FIG. 31. Each of the electrochemical cells comprises a microneedle working electrode 3122, a microneedle counter electrode 3124, and a microneedle reference electrode 3128, each of which is disposed on an electrical substrate 3130 and is electrically connected to control circuitry 3132 via electrical conductors 3134 (two conductors are numbered). Each microneedle working electrode 3122 comprises a layer of platinum or another working electrode base material 3135 with bound glucose oxidase, over which is applied a permselective or charge selective porous blocking membrane 3136. Each microneedle counter electrode 3124 comprises a layer 3135 of platinum, carbon or another counter electrode base material. Each microneedle reference electrode 3128 comprises a layer 3135 of silver/silver chloride or another reference electrode base material.

### Glucose Sensing Via Fluid Conductive Microneedles

In embodiments where electrochemical sensing is used to sense glucose via a microneedle-based sensing system, the sensing may be performed by microneedles that are tubular or “straw like”, which allows for bodily fluids containing the desired analyte to flow through the microneedles and into the housing of the microneedle array for measurement. The needles may be filled with any fluidic conducting materials (e.g., hydrogels) that establish a fluid conduction path both into and out of the microneedle housing through which analytes can diffuse. The diffusion of the analyte is due to changes in concentration of the analyte in the skin, so the fluid path should allow for movement in both directions to ensure consistency and accuracy between the concentrations in the body and the microneedle housing. The same fluidic conducting materials are employed inside the housing to present the analyte contained in the body fluid to the sensing elements. Electrochemical, opto-fluorescent or spectroscopy-based sensing elements can be employed to analyze the analyte once conducted into the body of the microneedle housing.

In electrochemical-based sensing, electrodes used to form the electrochemical cell(s) inside the sensor housing can be configured in multiple ways. The electrochemical cell(s) may be configured as an individual cell or in clusters to form an electrochemical sensing array to increase performance. The cells may be comprised of 2-electrode, 3-electode, 4-electrode, or other multi-electrode configurations, with common or isolated electrical circuits being used to create any number of sensing areas.

The chemically active materials, including the enzymes, that may be required to construct the electrochemical cell(s) can be coated directly onto a surface of the electrodes in the housing or may be mixed into the fluid conducting hydrogel materials.

Various methods can be employed to apply the electrode materials and chemically active components such as vapor deposition, electroplating, spraying or printing or using microelectronics fabrication methods.

Furthermore, various chemical components such as surfactants may be added to the fluidic conductive materials or hydrogels to help increase the flow of glucose or other analytes to the electrochemical cell inside the housing.

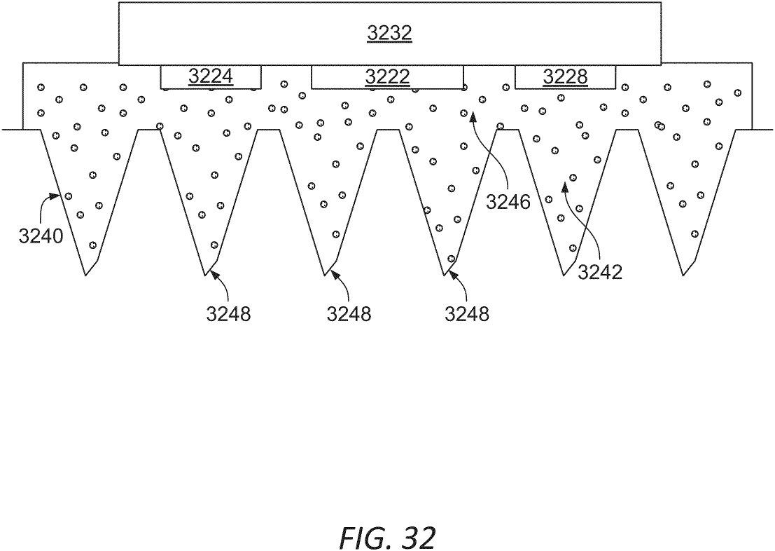

An embodiment of a hollow-microneedle-based sensing system with fluidic conductive materials that allow for diffusion of an analyte to the electrochemical cell electrodes of a 3-electrode electrochemical cell is illustrated in FIG. 32. The electrochemical cell comprises a working electrode 3222, a counter electrode 3224, and a reference electrode 3228, electrically connected to control circuitry 3232. The working electrode 3222 may comprises a layer of platinum or another working electrode base material with bound glucose oxidase, over which may be applied a permselective or charge selective porous blocking membrane as previously discussed. The counter electrode 3224 may comprise a layer of platinum, carbon or another counter electrode base material 3235, and the reference electrode 3228 may comprise a layer of silver/silver chloride or another reference electrode base material as previously discussed. The working electrode 3222, counter electrode 3224, and reference electrode 3228 are disposed in a reservoir defined by a housing. The reservoir contains a fluidic conducting material 3242 which allows for diffusion of an analyte (represented by dots 3246). A lower wall of the housing 3240 includes microneedles having hollow tips 3248 (three numbered) that open into the reservoir, which allows analyte 3246 in surrounding bodily fluids to flow through the microneedles and into the reservoir for measurement.

In some embodiments, opto-fluorescent sensing is used to sense analytes in a microneedle-based sensing system. In these embodiments, an opto-fluorescent sensing system is employed to measure analytes that flow into a sensor housing. As described above, fluidic conducting materials (e.g., hydrogels) may be used to establish fluid pathways for glucose or other desired analytes to flow from the body into a reservoir defined by the sensor housing for measurement. For opto-fluorescent sensing, the fluidic conducting materials contains a fluorophore and FRET acceptor/quencher molecules as described above. The opto-fluorescent fluorophore and FRET acceptor/quencher are bound or held in suspension inside the fluidic conducting materials and will react directly with the glucose or other selected analyte that the quencher is specific to. Inside the housing are light source(s) and detecting element(s) to excite the fluorophore and collect the fluoresced light that is returned. The light source(s) and detecting element(s) can be singular or in multiples. Where multiple light sources and/or detecting elements are provided, they may be arranged in lines, arrays or any functionally beneficial configuration to maximize the measurement performance. The measurement is made when light is used to excite the fluorophore that is bound in the hydrogel. For a direct glucose to light method, an increase in the returned light indicates an increased concentration of the analyte, whereas reduced or low light indicates a decreased concentration of the analyte. In addition to the magnitude of light, the amount of time required for the light emitted from the fluorophore to decay after being excited may also be measured to further establish the analyte concentration quantitatively.

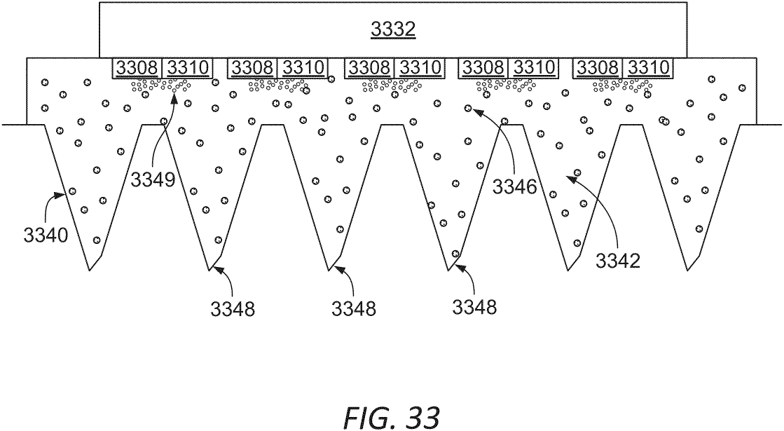

An embodiment of a hollow microneedle-based sensing system with fluidic conductive materials that allow for diffusion of an analyte to an opto-fluorescent sensing array is illustrated in FIG. 33. The opto-fluorescent sensing array includes a plurality of excitation light sources 3308 and a plurality of detectors 3310, which are electrically connected to control circuitry 3332. The excitation light sources 3308 and detectors 3310 are disposed in a reservoir defined by a housing 3340. The reservoir contains a fluidic conducting material 3342 (e.g., a hydrogel material) which allows for diffusion of an analyte (represented by dots 3346). The fluidic conducting material 3342 also contains fluorophore and FRET quencher molecules 3349, which are bound or held in suspension inside the fluidic conducting material 3342, and which interact directly with the glucose or other selected analyte to which the quencher is specific. A lower wall of the housing 3340 includes microneedles having hollow tips 3348 (three numbered) that open into the reservoir, which allows analyte 3346 in surrounding bodily fluids to flow through the microneedles and into the reservoir for measurement. Where the quencher is specific to glucose, the system is a direct sensing system, and an increase in the returned light indicates an increased concentration of glucose, whereas reduced or low light indicates a decreased concentration of glucose.

In other embodiments, an indirect opto-fluorescent sensing system is employed to measure analytes that flow into the sensor housing. As described above, fluidic conducting materials (e.g., hydrogels) are used to establish fluid pathways for glucose or other desired analytes from the body into the sensor housing for measurement. For opto-fluorescent sensing, the fluidic conducting materials contain fluorophores and FRET quencher molecules as described above. The sensor will be used to produce a measurable optical signal by using glucose and glucose oxidase to reduce the local concentration of oxygen through a chemical reaction. The sensor will produce a quantifiable light signal where the magnitude is in proportion to the oxygen concentration which is proportional to the glucose concentration. Or it may produce a time-dependent glucose signal where the length of time the sensor produces a light signal after being excited by a light source is proportional to the glucose concentration. Or both methods could be employed to calculate the glucose concentration. The light produced by the oxygen-sensitive FRET quencher-fluorophore combination bound in the gel along with glucose oxidase will fluoresce proportional to the concentration of oxygen presentation in the hydrogel. As described above, the light source and detecting elements can be arranged in a matrix or any suitable pattern to achieve the desired coverage area for illuminating and detecting the response from the fluorophore.

In addition, it may be desirable to add a separate set of open tipped microneedles that establish an isolated fluidic conducting pathway into an isolated sensor housing region for the purpose of measuring the concentration of oxygen in the body fluids without the oxygen concentration reduction effects from the reaction between glucose and glucose oxidase (GOX). This new zone would contain the same light source(s) and detecting element(s) and the hydrogel would contain the same oxygen-sensitive fluorophore and acceptor/quencher molecule. The difference would be the absence of glucose oxidase from the hydrogel or fluidic conductive materials. The measurement of the baseline levels of oxygen contained in the body will serve to increase the measurement accuracy of the analyte measurement zone. The baseline offset due to the natural levels of oxygen in the body can be subtracted from the analyte measurement values. In both sensing zones, glucose or other analyte is present and oxygen is measured. However, in the zone with the glucose oxidase, the oxygen concentration is dependent upon the glucose concentration.

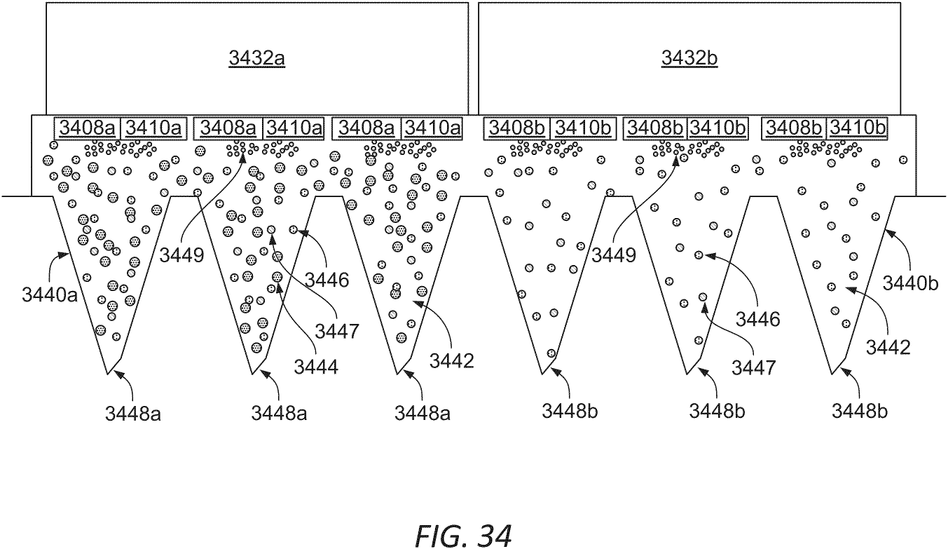

An embodiment of such an indirect opto-fluorescent sensing system is illustrated in FIG. 34. The indirect opto-fluorescent sensing system includes a first opto-fluorescent sensing array that includes a plurality of excitation light sources 3408a and a plurality of detectors 3410a, which are electrically connected to control circuitry 3432a. The excitation light sources 3408a and detectors 3410a are disposed in a first reservoir defined by a first housing 3440a. The first reservoir contains a fluidic conducting material 3442 (e.g., a hydrogel material) which allows for diffusion of glucose molecules (represented by dots 3446) and oxygen (represented by dots 3447) and which contains glucose oxidase (represented by dots 3444). A lower wall of the housing 3440a includes microneedles having hollow tips 3448a that open into the first reservoir, which allows analyte 3446 and oxygen 3447 in surrounding bodily fluids to flow through the microneedles and into the reservoir defined by the housing 3440a. The fluidic conducting material 3442 also contains fluorophore and FRET quencher molecules 3449, which are bound or held in suspension inside the hydrogel material 3442, and which interact directly with oxygen 3447 to which the quencher is specific. Thus, an increase in the returned light indicates an increased concentration of oxygen, whereas reduced or low light indicates a decreased concentration of oxygen. When glucose 3446 and oxygen 3447 are present, glucose oxidase 3444 catalyzes the oxidation of glucose 3446 with concomitant consumption of the oxygen 3447 to form gluconic acid and hydrogen peroxide. Thus, an increase in glucose will result in a decrease in oxygen, which will, in turn, result in a decrease in returned light to the detectors 3410a, whereas a decrease in glucose will result in an increase in oxygen, which will, in turn, result in an increase in returned light to the detectors 3410a.

The indirect opto-fluorescent sensing system of FIG. 34 may also include a second opto-fluorescent sensing array that includes a plurality of excitation light sources 3408b and a plurality of detectors 3410b, which are electrically connected to control circuitry 3432b. The excitation light sources 3408b and detectors 3410b are disposed in a second reservoir defined by a second housing 3440b. The second reservoir contains a fluidic conducting material (e.g., a hydrogel material 3442) which allows for diffusion of glucose molecules (represented by dots 3446) and oxygen molecules (represented by dots 3447). The fluidic conducting material 3442 (e.g., a hydrogel) also contains fluorophore and FRET quencher molecules 3449, which are bound or held in suspension inside the hydrogel material 3442, and which interact directly with oxygen molecules 3447 to which the quencher is specific. A lower wall of housing 3440b includes microneedles having hollow tips 3448b that open into the second reservoir, which allows glucose 3446 and oxygen 3447 in surrounding bodily fluids to flow through the microneedles and into the reservoir defined by the housing 3440b. Unlike the first reservoir, the second reservoir does not contain glucose oxidase. In this way, the concentration of oxygen 3447 in the body fluids can be measured without the oxygen concentration reduction effects arising from the reaction between glucose and glucose oxidase. As previously explained, the measurement of the baseline levels of oxygen 3447 contained in the body by the second opto-fluorescent sensing array will serve to increase the measurement accuracy of the glucose measured by the first opto-fluorescent sensing array.

In other embodiments, a spectroscopy-based sensing system may be employed to measure analytes that flow into a sensor housing. Spectroscopy uses electromagnetic (EM) radiation at various wavelengths to excite the molecule of a target analyte of interest into resonating. As described above, fluidic conducting materials (e.g., hydrogels) can be used to establish fluid pathways for glucose or other desired analytes to flow from the body into the sensor housing for measurement. For spectroscopy-based sensing, the fluidic conducting materials (e.g., hydrogels) serve to transport glucose or other analytes into the sensor housing for measurement by a spectroscopy source and detector. The spectroscopy source may be used to produce electromagnetic (EM) radiation at a specific wavelength or set of wavelengths. The wavelength(s) can be in the range of radiofrequency (RF) wavelengths all the way up and through visual light spectrum wavelengths to low energy ultraviolet (UV) wavelengths. The spectroscopy source may be a tuned set of RF antennae, an LED light source, a laser light source, or a combination of such sources. The spectroscopy-based sensing system may comprise a plurality of spectroscopy sources. The source wavelength(s) will be specified and tuned to excite particular analyte molecules. Depending on which analyte is measured, the wavelength of the electromagnetic radiation from the source(s) will be chosen to excite the analyte molecule into a vibrational state. The molecule will then produce a small amount of EM radiation at wavelengths that are both longer and shorter than the fundamental excitation wavelength. This is known as Raman scattering. The detector(s) can use filtering to mask out the EM radiation at the fundamental excitation wavelength, enhancing detection of lower intensity Raman scattering wavelengths. The fundamental excitation wavelength may be chosen to cause the most resonance in the desired analyte molecule which in turn will produce the greatest amount of Raman scattering. Additional EM radiation sources at other wavelengths may also be used to help further identify the concentration of analyte in the sample and will excite and/or resonate the analyte molecules at other wavelengths producing other Raman frequencies which can be used in further identification of the analyte molecules of interest.

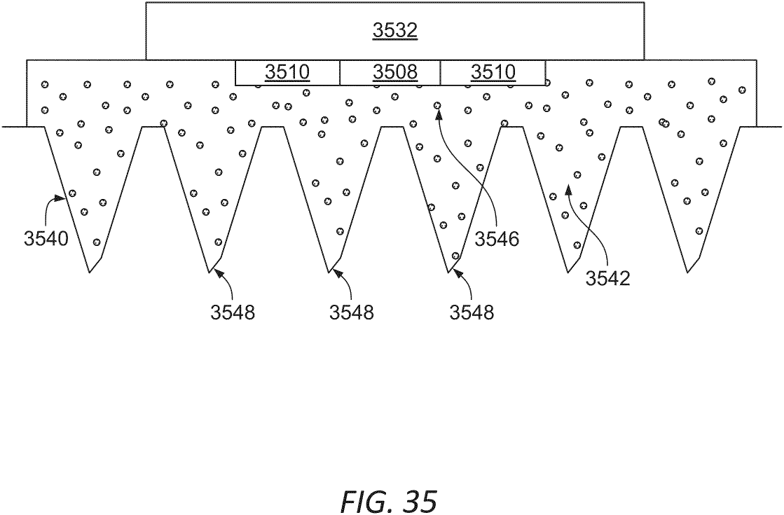

An embodiment of a hollow microneedle-based sensing system with fluidic conductive materials that allow for diffusion of an analyte to a spectroscopy-based sensing array is illustrated in FIG. 35. The spectroscopy-based sensing array includes an electromagnetic radiation source 3508 and two electromagnetic radiation detectors 3510, which are electrically connected to control circuitry 3532. The electromagnetic radiation source 3508 and detectors 3510 are disposed in a reservoir defined by a housing 3540. The reservoir contains a fluidic conducting material 3542 (e.g., a hydrogel material) which allows for diffusion of an analyte (represented by dots 3546). A lower wall of the housing 3540 includes microneedles having hollow tips 3548 (three numbered) that open into the reservoir, which allows analyte 3546 in surrounding bodily fluids to flow through the microneedles and into the reservoir defined by the housing 3540 for measurement.

In addition, to further increase the magnitude of Raman scattered EM radiation and increase the signal-to-noise ratio, a method of pre-orientation of the analyte molecule may be employed. The system can be constructed using a narrow analyte sensing zone inside the sensor housing in the area where the spectroscopy source(s) and detect element(s) are mounted. In this narrow zone, a set of electromagnetic plates, isolated coils, or a combination thereof may be installed to create an electromagnetic field capable of orienting the analyte molecules into a specific and consistent orientation prior to excitation by the spectroscopy EM source. This pre-orientation of the analyte molecules will serve to rotate the molecules into a more ideal orientation to increase the amount of energy that can be absorbed by the analyte molecules and thus allow the molecules to produce a larger amount of return Raman energy at “signature” wavelengths of the molecules.

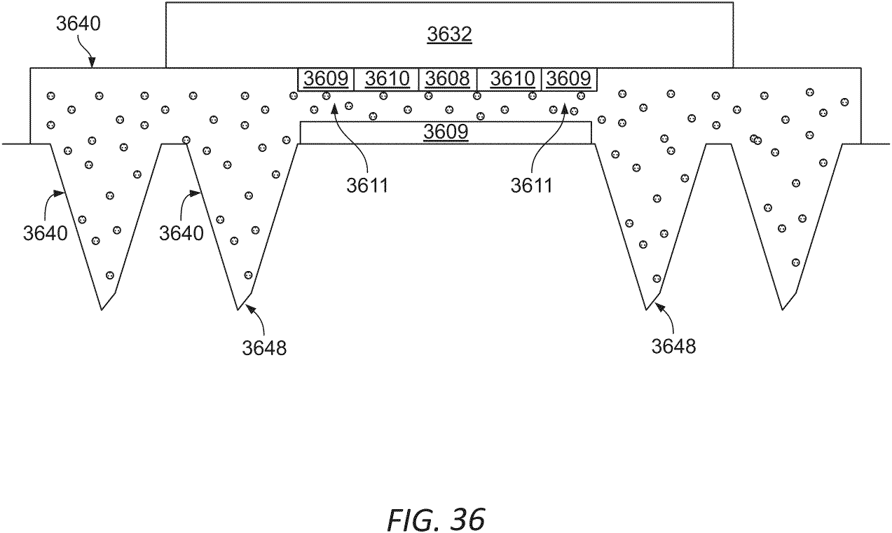

An embodiment of a hollow microneedle-based sensing system that allows for the orientation of analyte molecules in conjunction with detection by a spectroscopy-based sensing array is illustrated in FIG. 36. The spectroscopy-based sensing array includes an electromagnetic radiation source 3608 and two electromagnetic radiation detectors 3610, which are electrically connected to control circuitry 3632. The system also includes orientation elements 3609 (e.g., electromagnetic plates and/or coils), which form a sensing zone 3611 in which analyte molecules are oriented by polarization alignment. The electromagnetic radiation source 3608, detectors 3610, and orientation elements 3609 are disposed in a reservoir defined by a housing 3640. The reservoir contains a fluidic conducting material 3642 (e.g., a hydrogel material) which allows for diffusion and orientation of an analyte (represented by dots 3646). A lower wall of the housing 3640 includes microneedles having hollow tips 3648 (two numbered) that open into the reservoir, which allows analyte 3646 in surrounding bodily fluids to flow through the microneedles and into the reservoir defined by the housing 3640 for measurement. Additionally or alternatively, holes may be placed in one or both side surfaces of the microneedles to better allow bodily fluids to flow through the microneedles and into the reservoir for measurement.

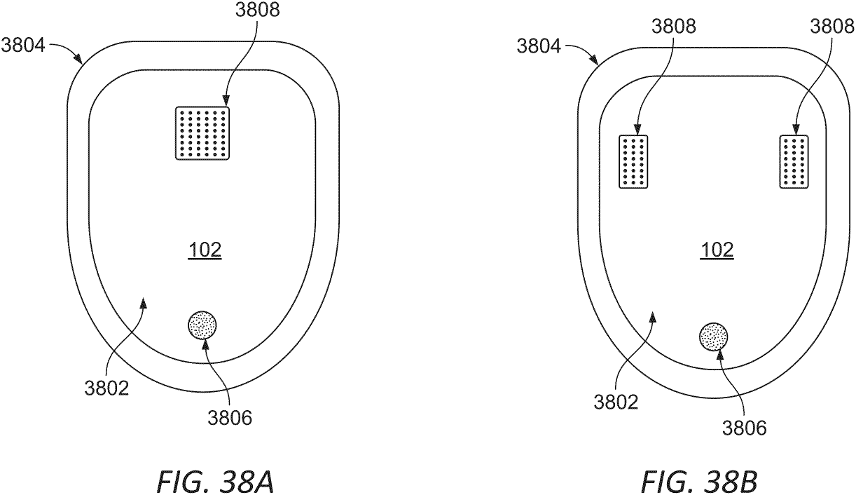

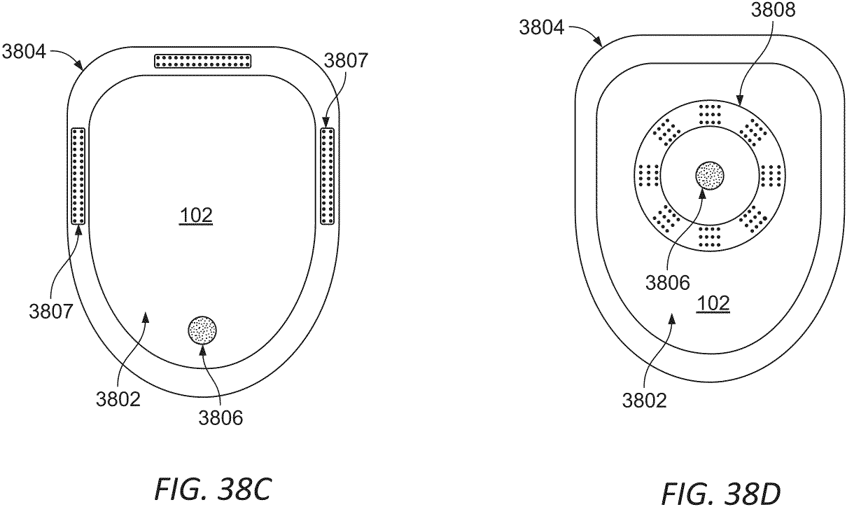

Any of the above-described microneedle sensing methods can be implemented in SPAID 102. FIGS. 38(A-D) illustrate different possible arrangements of the microneedles enabling both the sensing of glucose and the delivery of insulin. The figures are exemplary in nature and, as would be realized, other implementations may be viable and are contemplated as being within the scope of the invention. FIG. 38A shows a first possible embodiment. The figure shows a bottom view of SPAID 102 showing base 3802 which, when SPAID 102 is deployed, rests on the skin of the user. Adhesive 3804 serves to adhere SPAID 102 to the user’s skin. The liquid drug delivery point is indicated by reference 3806 and may be any one of a number of delivery methods which will be discussed later herein. A microneedle sensing array 3808 is shown disposed on the base 3802 of SPAID 102 such that when SPAID 102 is mounted on the skin of the user, the microneedle array 3808 contacts the user’s skin and uses one of the above-described methods to measure the glucose level of the user. Several microneedle structure suitable for use with the described embodiments are described in U.S. Pat. Application 63/154,003, filed Feb. 26, 2021, the contents of which are incorporated herein it their entirety.

A first variation of this embodiment is shown in FIG. 38B wherein dual microneedle sensing arrays 3808 are disposed on the base 3802 of SPAID 102. FIG. 38C shows a third variation in which multiple microneedle sensing arrays 3808 are disposed within the adhesive 3804 (or on the underside of a tray) which adheres SPAID 102 to the body of the user. Lastly, FIG. 38D shows yet another variation in which microneedle array 3808 is shown in circular form and is disposed on the base 3802 of SPAID 102. Note that in the embodiment shown in FIG. 38D, liquid drug delivery point 3806 is disposed in the center of the circular microneedle sensing array 3808. However, liquid drug delivery point 3806 may be disposed outside of the circular microneedle sensing array 3808.

To reduce the influx of molecules that may be undesirable in the sensor housing when using open tipped fluidic conducting microneedle sensing array(s) 3808, it may be necessary to add a means of filtering or blocking such molecules. One possible implementation would be to use a membrane such as Nafion® or other molecular selective membranes that have pore sizing suitable for glucose (or other desired analytes) while rejecting larger molecules that may be undesirable to have in the measurement housing.

Alternatively, it may be possible to use electrically conductive coatings or electrodes to create an electric field in close proximity to the tips of the microneedles of microneedle sensing array(s) 3808 that would serve to pull or direct undesirable charged molecules away from the tips of the microneedles while in the body. The electrodes would collect the molecules on the outer surface of the microneedles if coated in the electrically conductive materials, thus preventing ingress into the microneedle sensor housing. In addition, there could be an additional set of electrodes inside the sensor housing to further filter should some undesirable charged molecules enter into the microneedles.

The electrodes would be driven by the electronics of SPAID 102 and would be capable of producing fixed DC voltages and or cycled AC or pulsed voltages. If required, the voltage source may also be isolated with respect to any other electrodes in the system in particular if an electrochemical measurement system is employed inside the measurement housing.

One possible installation method for a glucose-sensing microneedle sensing array(s) 3808 requires the user to firmly place and press the base surface 3802 of SPAID 102 against the skin in the recommended body locations. To further enhance the microneedle penetration into and through the stratum corneum, an ultrasonic transducer (not shown) can be used to vibrate the body of the microneedle sensing array(s) 3808 temporarily and periodically. The ultra-sonic vibrational energy serves to agitate and further deepen microneedle sensing array 3808 to the desired maximum depth controlled by the needle length. This is in conjunction with pressure being applied by adhesives 3804 that will hold the base 3802 of SPAID 102 firmly to the skin of the user and the skin pressure that was established during the initial installation by the user that is creating an upward pressure force towards the base 3802 of SPAID 102 and against microneedle sensing array(s) 3808. Adhesive 3804 is preferably placed in close proximity to and all around the microneedle sensing arrays 3808 and the microneedle sensing arrays 3808 may be mounted slightly proud of the base. In some embodiments, an applied force, such as from a spring, from foam material or from another force system may be provided, from the interior of the housing onto the back of the microneedle array to ensure and maintain proper downforce of the microneedle array onto the surface of the skin.

### Spectrographic Sensing Methods

Glucose sensing can be performed in SPAID 102 by using a spectroscopy source and detector system mounted in the base of the housing of SPAID 102 to measure glucose or other analytes directly through the skin. Alternatively, SPAID 102 could use a photon-based spectroscopy system to measure glucose or other analytes directly through a light-conducting cannula that is inserted into the skin that may or may not also be used for insulin delivery. SPAID 102 could comprise a spectroscopy-based sensing system combined with an inserted insulin delivery cannula. Alternatively, SPAID 102 could comprise a spectroscopy-based sensing system combined with a microneedle-based insulin delivery system. Alternatively, SPAID 102 could comprise a spectroscopy-based sensing system combined with a transdermal insulin delivery system.

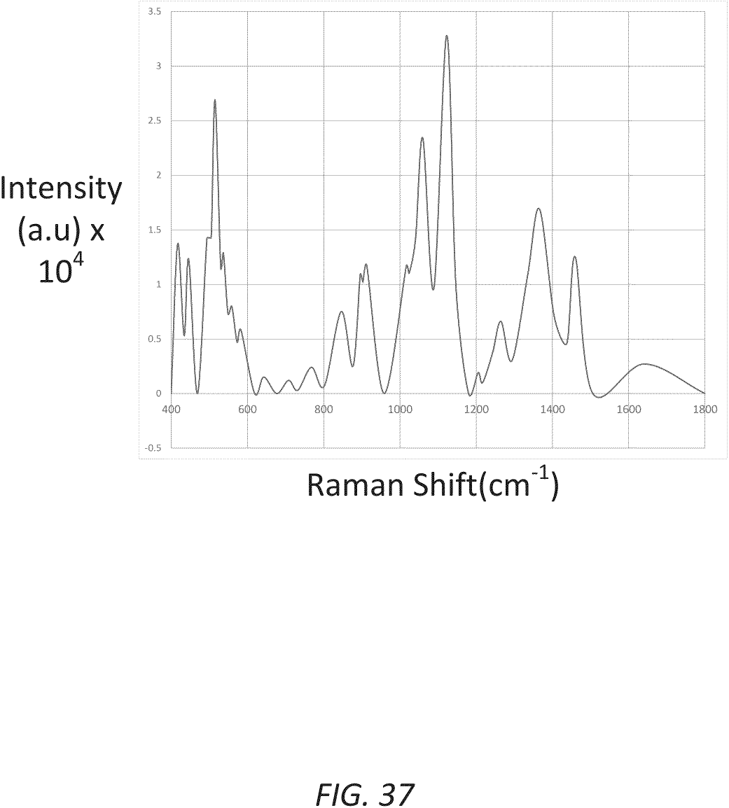

As previously noted, spectroscopy uses electromagnetic (EM) radiation at numerous wavelengths to excite the molecule of a target analyte of interest into resonating. For glucose sensing, the glucose molecule when excited by a source of EM radiation will in turn resonate and return EM radiation at other wavelengths that are both longer and shorter than the original excitation wavelength. The return EM energy due to the resonance of the molecule is called Raman scattering and is the essence of what is known as Raman Spectroscopy. Glucose molecules once excited will resonate at specific wavelengths and this pattern of specific wavelengths and magnitudes forms a “fingerprint” that is specific to the glucose molecule. The greater the magnitude at those specific wavelengths, the greater the number of glucose molecules that are resonating in the sample. Therefore, the magnitude information can be used to determine glucose concentration in a sample fluid or in the body. FIG. 37 shows a typical “fingerprint” plot of resonated glucose energies after the glucose has been excited with near IR light energy.

Some of the main challenges associated with taking measurements on the body are interference with other molecules that are also present and resonating or scattering the source EM radiation. An additional issue is attenuation of both the sourced EM radiation and the returned resonated EM energy. In the case of an external body measurement of glucose, the EM radiation used to excite the glucose molecule is typically near field infrared light (IR) which travels through the skin to excite glucose molecules in both the interstitial fluid as well as in whole blood in the capillaries and veins. Any molecules that comprise the layers of skin, such as water, salts, amino acids, proteins, and other molecules, can either attenuate the near IR light or cause additional resonating energies. In addition, once the glucose molecules receive the energy, the resonated energy specific to the glucose molecules needs to travel back out of the skin to be detected. The return energy is quite small in magnitude and can also experience similar attenuation due to the layers of skin and tissue and cause additional secondary interactions with other molecules at new resonating frequencies. Therefore, the closer the system can get to sampling a pure analyte with a direct path for both the excitation and resonated energy to travel back and forth from the source to the detector, the more accurate the system will be.

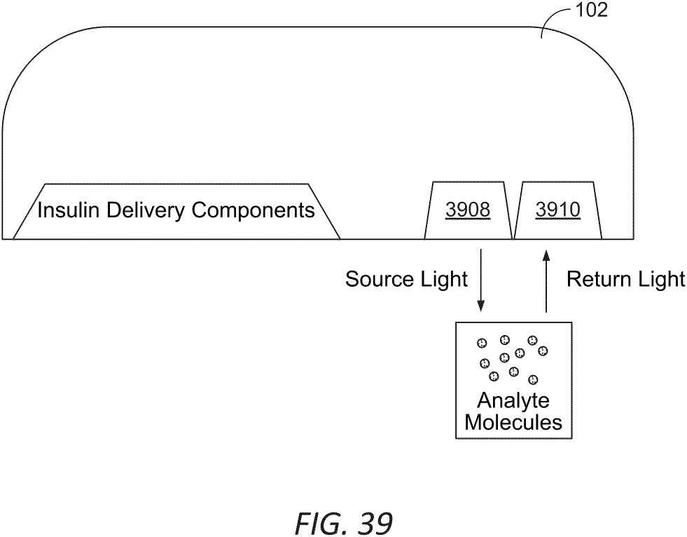

FIG. 39 shows one embodiment of SPAID 102 wherein the glucose-sensing is performed using a spectroscopy-based system to measure glucose through the skin. In this embodiment, the source 3908 and detector 3910 for the spectroscopy system are mounted in the base of the housing of SPAID 102. The EM energy is generated by source 3910 and directed down from the base of SPAID 102 into the skin. The EM energy can be in the form of UV, visible or infrared light as well as near, mid, or far IR, and RF energy. The glucose molecule can be made to vibrate using EM energy throughout this whole range of wavelengths. The wavelength(s) used by the source are maximized to deliver the most EM energy to the glucose or other analyte while penetrating the necessary layers of skin, depending upon the mounting location of SPAID 102. The receiver 3910 is tuned to receive the signature Raman EM energy released by the glucose molecule when excited by the source EM energy.

By way of example, if 532 nm green visible light is used as the source EM energy, EM light source 3908 may be a miniature laser, a light-emitting diode or other light sources capable of operating in a narrow band of wavelengths around 532 nm. The source EM energy is focused on the skin using a lens and / or other optical components to concentrate and maximize the excitation energy to the region of interest in the skin. Other wavelengths may be used.

The returned Raman vibrational energy is collected using a detector 3910 which may be, for example, an array of photodetectors, lenses and mirrors, or other opto-electrical components necessary to gather the return energy photons. The use of a prism or other light filtering device is employed to selectively return energy photons of a specific wavelength or of wavelengths that correspond to the Raman spectroscopy signature for the specific excitation energy. The system can be tuned to focus on the return energy of specific wavelengths that match peaks of energy found in the Raman spectroscopy signature for the given analyte. The selectivity of the filter can improve the signal-to-noise ratio of the system, thereby ensuring response at peaks relative to the measured analyte.

The resulting electrical signal from the photodetectors are amplified as needed and then digitized using an analog to digital converter with enough resolution and sampling speed to meet the requirements of the desired system output. In the case of a glucose measurement, the system may need to respond in 0.1 mg/dL increments over the range of 0 - 400 mg/dL thus requiring 4000 steps of resolution. Thus, preferably a 12 bit or higher resolution A/D converter may be used. Alternatively, the system could use an A/D converter of lesser resolution and sample faster than required and employ averaging in the software.

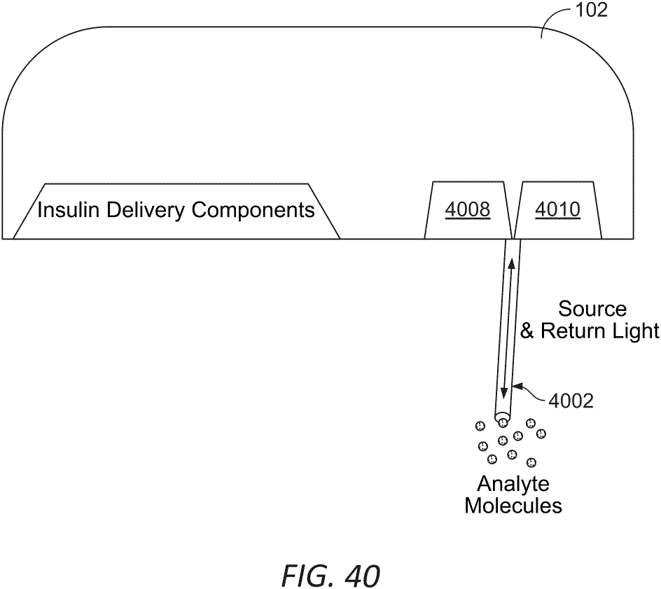

FIG. 40 shows a second embodiment using spectroscopy, in which glucose molecules are indirectly measured by exciting a secondary molecule with EM energy via light conductor 4002 that is inserted into the skin. Light conductor 4002 may comprise, in various embodiments, a fiberoptic plastic, glass or other light conducting material. The secondary molecule could be, for example, hydrogen peroxide, oxygen or any other molecule that can be produced in direct proportion to the glucose concentration in the body.

If hydrogen peroxide is used it will be produced via a glucose-to-glucose oxidase reaction. In this embodiment, the glucose oxidase enzyme is directly coated onto the tip of cannula 4002 by possibly binding it within a coating or structure of the cannula itself or possibly in a suspension of a gel matrix. Once inserted under the skin in the subcutaneous region, glucose in the interstitial fluid will react with the glucose oxidase and create hydrogen peroxide directly at the tip of light conductor 4002. This localized production of hydrogen peroxide is excited by the sourced EM energy produced by light source 4008 that travels from SPAID 102 to the tip 4020 of light conductor 4002. Because the peroxide is locally produced at the tip of light conductor 4002, there will be little to no interference or attenuation of the sourced and resonated return Raman EM energy from the hydrogen peroxide molecule. The return Raman EM energy is detected by detector 4010.

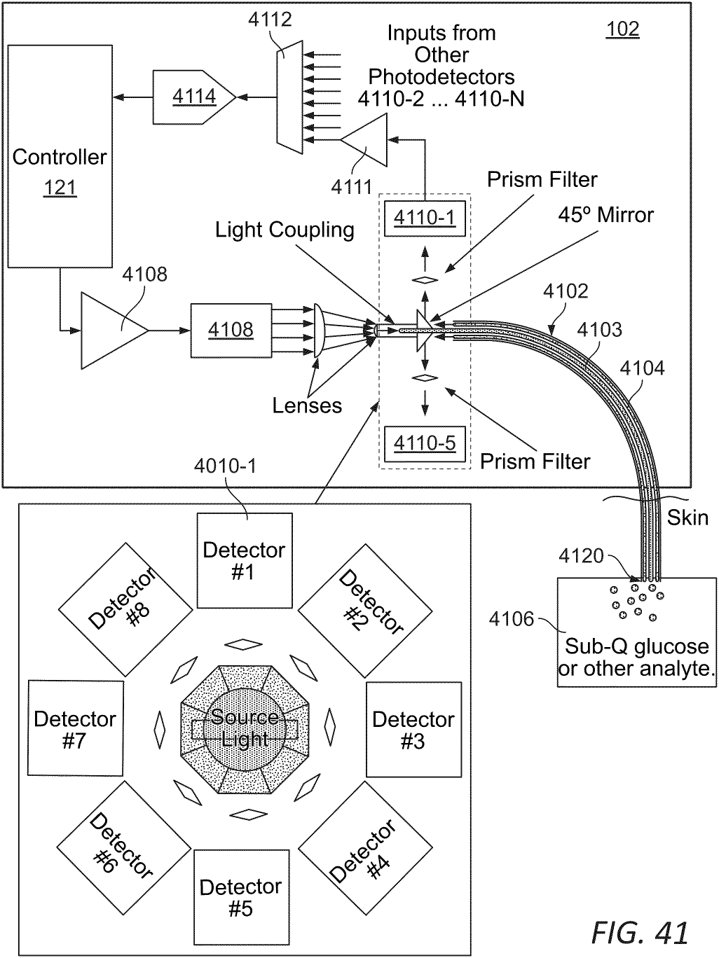

The block diagram in FIG. 41 shows one possible implementation a of glucose sensing system using spectroscopy through a cannula 4102. The system uses dual lumen cannula 4102 comprising light conductors 4103, 4104 constructed in a coaxial format. The inner light conductor 4103 passes the excitation light to the target from the excitation source 4108 inside the housing of SPAID 102. The excitation source 4108 can be a laser, LED, or other sources capable of producing the EM energy source at the wavelength, spectrum, and intensity required to excite the target analyte. The excitation source energy may pass through various lenses, mirrors, prisms, or other elements necessary to properly direct the energy to and through the inner lumen. The outer light conductor 4104 is in the form of a coaxial tube with the inner light conductor 4103 passing therethrough. The walls of the outer light conductor 4104 both inside and out may be covered in a blocking cladding to prevent EM energy leakage out of the system or between the two light conductors 4103, 4104.

As the excitation EM energy passes down to the target analyte 4106, the analyte will become excited and produce vibrational Raman EM energy in return that is both of longer and shorter wavelengths than the original excitation. The return energy will travel back to SPAID 102 via the outer light conductor 4104 to reflecting mirrors (e.g., 45° reflecting mirrors), through selective filter prisms, and to photodiode detectors 4110-1 ... 4110-N or other detectors sensitive to the return Raman EM energy. The prisms are useful for selecting and directing the specific Raman EM energy to the detectors that match the “fingerprint” energy spectrum of the desired analyte. Alternatively, the prisms could be manipulated or moved to direct energies of other wavelengths that are also part of the “fingerprint” energy spectrum of the desired analyte. Additionally, some of the prisms could be tuned for one portion of the analyte spectrum with others tuned for to senses different wavelengths to determine the concentration of the target analyte.

The remaining components are required to drive the source and measure the return energy. A programable amplifier 4108 allows the microprocessor to control the power and thus the intensity of EM energy source as well as output on and off control and pulsing. The photodiode amplifier 4111 increases the magnitude to the electrical signal from the photodiode that converts the return Raman EM energy into an electrical current. The multiplexor 4112 is used to select one of the signals from the photodiode amplifiers 4110. The ADC 4114 digitizes the analog signals from the MUX 4112 and feeds the data to controller 121 for analysis.

As shown in FIG. 41, the light source 4108 for the excitation energy may be located inside the housing of SPAID 102 and can be collimated and directed through light conductor 4103 to tip 4120 of cannula 4102 using a combination of mirrors, lenses and other optical elements. Similarly, the return resonated Raman light energy will be directed through a similar series of mirrors, lenses, prisms, filters, and other optical elements to multiple photodetectors 4110-1 ... 4110-N. The output of the photodetectors 4110-1 ... 4110-N are multiplexed by multiplexor 4114, then pass through amplifiers, and an analog-to-digital converter and to controller 121 running MDA 129 (and/or user app 160 executing on user device 105) which analyzes the collected signal.

The inset portion of FIG. 41 shows the arrangement of multiple photodetectors 4110-1 ... 4110-N. In this embodiment, 8 photodetectors are shown; however, as would be realized, any number of photodetectors may be used.

One key aspect is the construction of the tip 4120 of cannula 4102 that is inserted into the body and interstitial fluid. In one possible embodiment, tip 4120 comprises a Fresnel lens. The light from the surface of the Fresnel lens is all returned to a single point. For the measurement of hydrogen peroxide concentration in proportion to glucose, the lens surface will be coated with glucose oxidase by binding the GOX with the lens material or other binder or coating or, alternatively, with a hydrogel. The production of hydrogen peroxide on the surface of the lens allows for both receiving the excitation EM light energy and for returning the vibrational Raman energy.

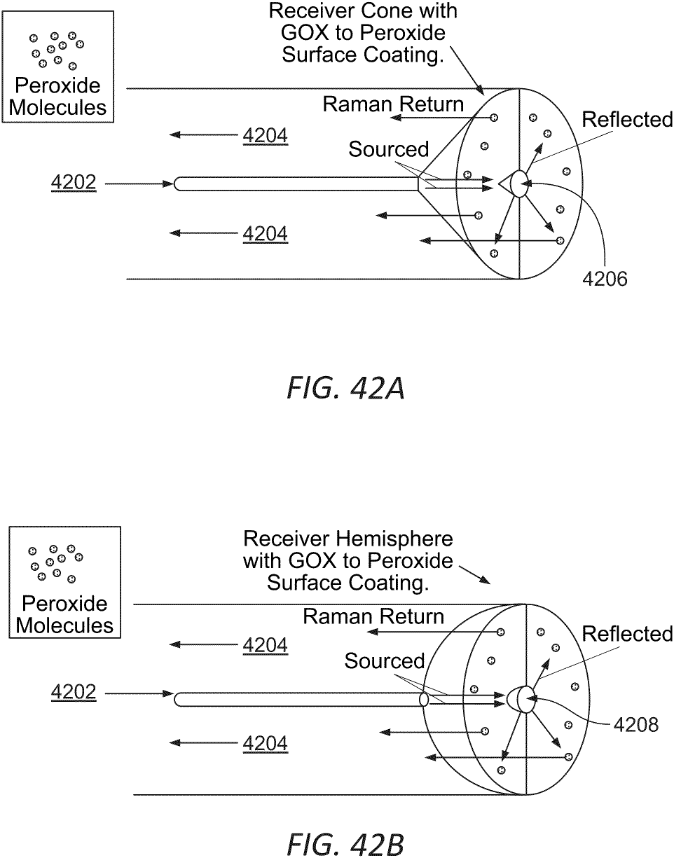

Alternative cannula tip shapes, as shown in FIGS. 42(A-B) may be used to direct the sourced EM energy and return the Raman resonated energy. One possible shape could be a cone shape 4206 as shown in FIG. 42A with the light being sourced down the center tip 4202 of the cone 4206 and the return Raman energy travelling up a secondary return lumen 4204. Another alternative cannula tip shape 4208 is shown in FIG. 42B as a hemisphere shape with the light being sourced down the center 4202 and the return Raman energy travelling back via secondary return lumen 4204.

In both of the above embodiments of FIGS. 42(A-B) (cone-tipped 4206 and hemisphere-tipped 4208) the inner surface of the return lumen 4204 may be coated or bound with GOX. The sourced light travels down the center inner lumen 4202 and reflects off of the suspended reflector cone 4206 or hemisphere 4208 allowing the EM energy to radiate in all directions back towards the GOX coated inner cone 4206 surface or hemisphere 4208 surface. The energy subsequently reacts with the produced hydrogen peroxide which will radiate its Raman return energy up the coaxial outer lumen 4204 back to the detector 4010 mounted in the housing of SPAID 102.

### Insulin Delivery Methods

SPAID 102 includes a delivery patient interface 186 for interfacing with the user to deliver the liquid drug(s) from either or both of reservoirs 124, 124-2 to the user. Various embodiments of the delivery patient interface 186 will now be discussed. Any one of the disclosed embodiments of the delivery patient interface 186 may be combined with any of the previously-discussed methods of glucose detection (electrochemical sensing, opto-fluorescent sensing, and spectrographic sensing) or any other method of sensing not discussed herein within a SPAID 102.

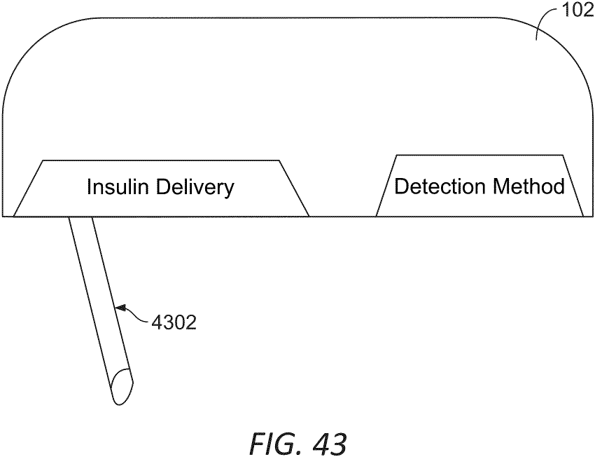

In one embodiment shown in FIG. 43, delivery patient interface 186 comprises a cannula for delivering the liquid drug into the body of the user (which may be done subcutaneously, intraperitoneally, or intravenously). In the case where the delivery patient interface 186 is a cannula, SPAID 102 may further includes an insertion mechanism 188 for inserting the cannula into the body of the user, which may be integral with or attachable to SPAID 102. The insertion mechanism 188 may comprise, in one embodiment, an actuator that inserts a needle and cannula 4302 under the skin of the user and thereafter retracts the needle, leaving cannula 4302 in place. After use, insertion mechanism 188 may remain within SPAID 102 or, if attachable to SPAID 102, may be detached therefrom. At the end-of-life of SPAID 102, cannula 4302 may be retracted from under the skin of the user by an actuator (not shown) which may be, for example, a spring-loaded retractor, before SPAID is removed from the body of the user. This retraction actuator may be part of the insertion mechanism 188 or may be separate therefrom.

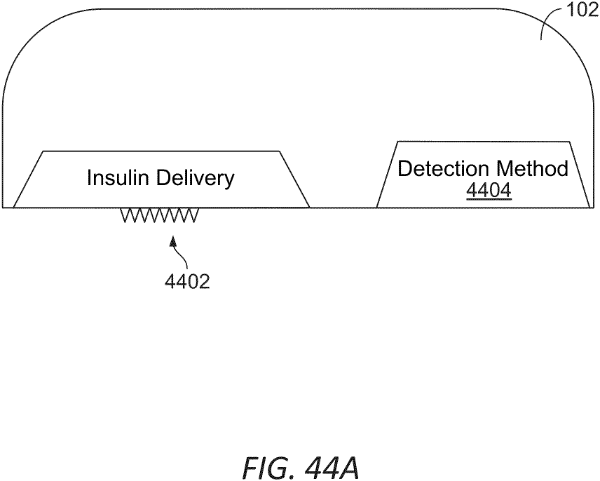

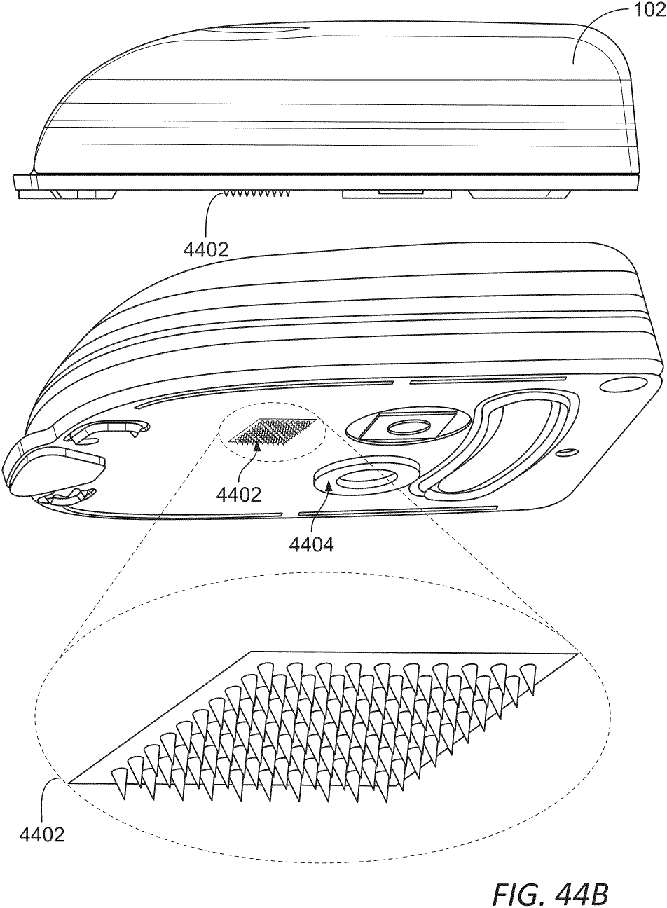

In another embodiment of a delivery patient interface 186 is shown in schematic form in FIG. 44A, one or more arrays of microneedles 4402 may be used to deliver the liquid drug. FIG. 44B shows a concept image of this embodiment, showing an enlarged view of the microneedle array 4402 in the inset. Also shown is detection method 4404, which is shown here as a wireless communication antenna, such as a nearfield antenna, although any detection method may be used with the microneedle array 4402 method of delivery.

The delivery of insulin can be performed by using an array of solid tipped microneedles. The microneedle array may be coated with insulin bound to a charged molecule or other binding molecules and/or use an electric field to hold the insulin onto the surface of the solid microneedles. In some embodiments, the needles are covered in a grid of electrical conduction plates which may be each individually connected to the control electronics. Each plate is capable of holding the surface coated insulin molecules which are bound via an electric field. The size of the conduction plate is directly proportional to the amount of bound insulin molecules on its surface. The size of the plate is design specific and may be sized to meet the minimum quantity or dose of insulin desired to be released. When the microneedle array is installed onto the skin and is in contact with interstitial fluid, the insulin molecules will remain bound to the microneedle surface via the electric field generated by the conduction plates and the bound charged molecule. When there is a demand from the control system to deliver insulin into the skin, the electric field associated with an individual plate, or a portion of an individual plate, is removed, allowing the insulin to be released from the needle surface of that portion or of that plate and be diffused into the interstitial fluid and subsequently into the body. The amount of insulin released from a single conduction plate may be designed to meet the smallest insulin dose required. If a larger dose is required then additional conduction plates or additional portions may have the electrical charge removed, thereby releasing additional insulin payload. This release process can continue until all of the conduction plates have had their insulin released.

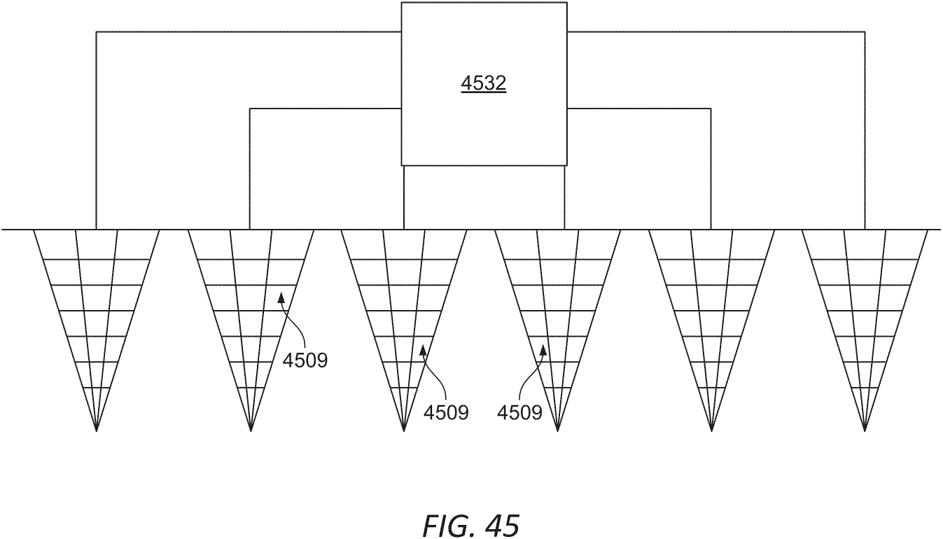

The release of insulin can be controlled and triggered by an AID control system which is receiving glucose level information from a glucose sensor and is making the subsequent insulin delivery decisions to maintain glycemic control. An embodiment of such a system is shown FIG. 45, which is a schematic illustration of a microneedle array in which each microneedle is covered in a grid of individual conduction plate sections 4509 (three numbered), which are in turn coated with insulin (not numbered) and which are individually connected to a control electronics 4532.

Alternatively, to support the charge-based release process, the tip of each needle may be held at ground potential for the system or at an opposite charge value to the conduction plates. This would allow for a small current to flow across each needle, pulling the molecules in a controlled manner towards the tip and driving the molecules deeper into the skin for more rapid diffusion.

In other embodiments, the system is configured to provide a charge only in conjunction with the release of the molecules, rather than using a constant charge to hold the molecules that is removed to release the molecules. Further options include employing a pulsing or duty cycling of the charge to force proportionate release of bound molecules. This could be an additional method of release control, rather than all or nothing release from individual plates. For example, each whole needle may correspond to one coated plate, and a timed pulsing, or duty cycling, may be used to drive the molecular release process.

In some embodiments, the delivery of insulin can be performed by using an array of hollow-tipped microneedles. The array may be connected to an internal chamber or reservoir in a housing of the device where body fluids such as interstitial fluid can freely diffuse in and out. Each of the microneedles as well as the internal chamber are filled with a hydrogel matrix or other fluidic conductive medium. Inside the housing, one or multiple tubes or cannulae connect the hydrogel matrix to an insulin delivery control system. The control system may include, for example, a reservoir for insulin, a pump (electrical or mechanical), fluid control valves, a microprocessor-based control system, a power source, various system feedback mechanisms, and user interface controls and communications systems. When insulin is required, for example, by a time-based delivery protocol, by user demand, by CGM demand or other insulin control criteria, the insulin is released and or pumped into the gel matrix inside the housing chamber. The insulin will then flow through the gel matrix and into the body by means of diffusion. Since the concentration of insulin will be higher inside the microneedle housing than in the body, the diffusion process will serve to equalize the difference and thus cause the insulin to flow out of the housing chamber through the microneedles into the skin.

The microneedles may be of any suitable size, for example, ranging anywhere from 100 to 2000 microns in length, depending on the skin thickness and the need to reach into the body to connect with the interstitial fluid layers. The outer diameter may range, for example, from 50 to 250 microns, with the needle shape beneficially being cone-like and narrowest at the tip where it is inserted into the skin. The internal diameter may range, for example, from 10-40 microns at the tip. The microneedle array may be arranged in any suitable pattern. One possible configuration could be a 10 x 10 square grid array with the spacing between needles ranging, for example, from 500 to 2000 microns, although many other patterns and spacings are viable options as well.

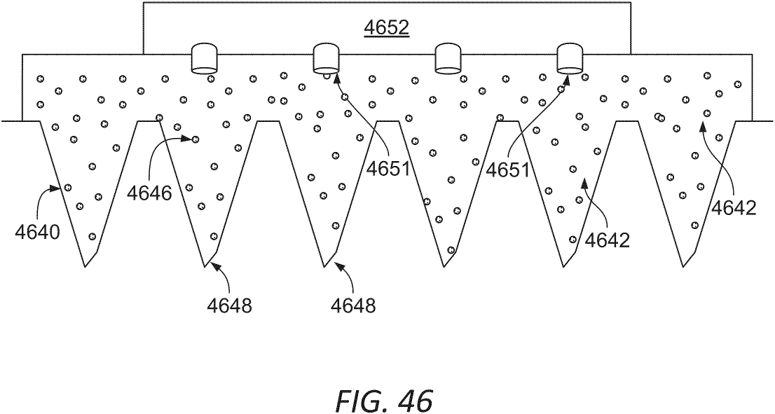

An embodiment of a hollow-microneedle-based delivery system is illustrated in FIG. 46 in which an internal housing chamber/reservoir is filled with a hydrogel matrix 4642 or other fluidic conductive media, which allows for diffusion of insulin (represented by dots 4646). A lower boundary of the housing chamber 4640 includes a plurality of microneedles having hollow tips 4648 (two numbered) that open into the internal housing chamber. A plurality of insulin delivery ducts or tubes 4651 fluidically connect the hydrogel matrix 4642 to an insulin delivery control system, which may include an insulin reservoir and pump system as described above, among other components. Once delivered into the housing chamber, the insulin 4646 will then flow through the gel matrix 4642 and out of the hollow tips 4648 of the microneedles.

As previously noted, the delivery of insulin can be performed by using an array of hollow tipped microneedles using diffusion pressure to drive the transport of insulin from areas of high concentration in a housing chamber to low concentration in the skin and body. Additionally, the transport of insulin out of the housing chamber and into the body can be enhanced or accelerated by using iontophoresis to push or pull the insulin through the hydrogel from inside of the housing chamber to the exterior of the microneedles in the skin. This may be achieved by setting up a constant or varying electrical field using electrical conductors that are mounted between the region in the housing chamber where the system delivers insulin to the housing chamber and an exterior tip of each microneedle. Depending on the pH of the skin and the charge state of the insulin molecules, a specific voltage or sets of voltages with different pulse profiles or static voltage levels may be employed. The resulting electrical field will serve to push or pull the insulin molecules from inside the housing toward the exterior tip of the microneedles. Some of the microneedles in the array may also be configured as pH sensors to provide feedback to the system and thus alter the field strength or time to achieve the desired system performance. Once sufficient insulin has been driven to the exterior of the microneedles, the iontophoresis system can be turned off for a period of time allowing for bodily diffusion to take over. The iontophoresis of insulin out of the housing chamber and into the body will decrease the delay from the time when an insulin delivery demand is received by the SPAID system to the time when the body receives the insulin. This system will not affect the quantity of insulin delivered but instead, it will control how quickly the measured insulin dose enters the body.

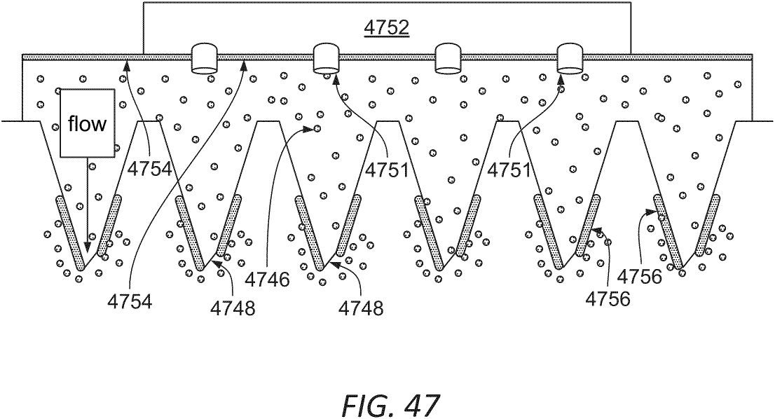

An embodiment of an iontophoresis-based delivery system is illustrated in FIG. 47 in which an internal housing chamber is filled with a hydrogel matrix 4742 or other fluidic conductive media, which allows for diffusion of insulin (represented by dots 4746). A lower boundary of the housing chamber includes a plurality of microneedles having hollow tips 4748 (two numbered) that open into the internal housing chamber. A plurality of non-conductive insulin delivery ducts or tubes 4751 fluidically connect the hydrogel matrix 4742 to an insulin delivery control system 4752, which may include an insulin reservoir and pump system as described above, among other components. Once delivered into the housing chamber, the insulin 4746 can then flow through the gel matrix 4742 and out of the hollow tips 4748 of the microneedles. The iontophoresis-based delivery system further includes a first electrical conductor 4754 (sometimes referred to herein as an electrode), which is positioned at an upper boundary of the housing chamber and which acts as a first pole of the iontophoresis system. The first electrical conductor 4754 may be in the form of a continuous conductive surface placed between the non-conductive insulin delivery ducts or tubes 4751. A remainder of the housing interior is non-conductive as well. A plurality of second electrical conductors 4756 (which may also be referred to as an electrode) are placed at an exterior tip of each microneedle. All of the plurality of second electrical conductors 4756 may be electrically connected together such that they act as a second pole of the iontophoresis system. A remainder of the exterior of the microneedles and the bottom (skin side) surface of the housing may be electrically isolated and non-conductive. Upon application of a suitable voltage between the electrical conductor 4754 and the plurality of second electrical conductors 4756, an electric field is created such that insulin molecules 4746 will migrate out of the needle tips 4748 and collect near the exterior electrodes. The electrical field may be powered off periodically to allow the migrated insulin molecules to diffuse into the body.

In alternate configurations of the iontophoresis-based delivery system, a third pole is provided. For example, the electrical conductors 4756 of every other (alternating) microneedle may be connected electrically together to form second and third poles of the iontophoresis-based delivery system. This arrangement could be used to create an electric field between adjacent microneedles that could be further used to force prior collected insulin molecules from the surfaces of the microneedles and into the body. In general, the amount of electrical current is controlled so as to not cause patient discomfort and or exceed regulatory requirements.

As previously indicated, the delivery of insulin can be performed with an array of hollow-tipped microneedles using diffusion pressure to drive the transport of insulin from areas of high concentration in the housing to areas of low concentration in the skin and body. Additionally, the transport of insulin out of the housing and into the body can be enhanced or accelerated by using ultrasonic sonophoresis. The ultrasonic sonophoresis will vibrate or agitate the insulin molecules through the hydrogel from inside of the housing to the tip of the microneedles and further drive the insulin into the deeper layers of the skin more rapidly. This may be achieved by the use of an ultrasonic transducer that is mounted on an inside of the microneedle housing near the insulin delivery tubing in direct contact with the interior hydrogel matrix. The vibrations produced by the ultrasonic transducer vibrate the microneedle housing, hydrogel, and needles themselves. This vibration serves to increase the transport of the of the insulin molecules, in a process known as sonophoresis, through the hydrogel, out the tips of the hollow microneedles and deep into the layers of skin, where the body will absorb and use the insulin. The ultrasonic sonophoresis system acts to open the skin tissue and pores located under the microneedle array to allow for the physically large insulin molecule to diffuse with greater ease and speed into the skin. The insulin molecule is approximately 5,800 Da (Daltons) in size. Typical low frequency ultrasonic sonophoresis systems in the range of, for example, 20 kHz to 120 kHz, can allow for transport of molecules in the range of 25,000 Da to 50,000 Da directly through the exterior skin. The use of the microneedles combined with ultrasonic sonophoresis will greatly increase the diffusion rate and consistency.

In addition, a common problem with open tipped microneedles is clogging due to a buildup of bodily wound healing components such as proteins, blood cells, antibodies and macrophages. These component over time may block the openings as the tip of the microneedles preventing the diffusion of insulin. The periodic use of the ultrasonic sonophoresis system may serve to disrupt the formation of these components and allow for the insulin delivery pathway to remain open and viable to diffusion.

An additional embodiment would be the combination of the previously described iontophoresis system with the use of the sonophoresis system, both mounted inside the microneedle housing. These two systems can serve together to increase the speed and efficiency of the insulin transfer as well as duration for which the complete SPAID 102 can remain viable and effective in its location on the body.

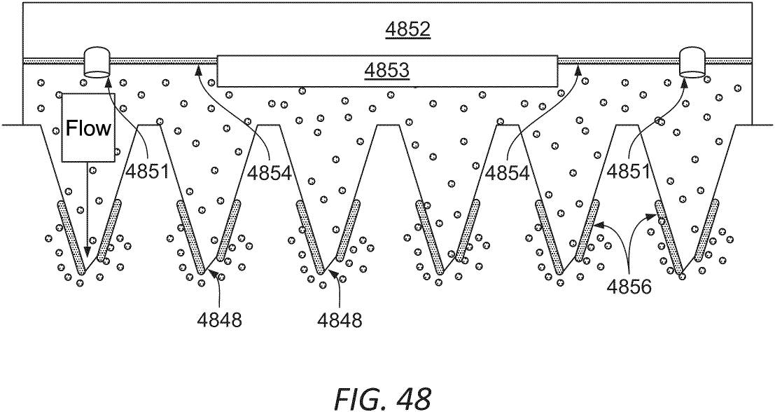

An embodiment of such a delivery system is illustrated in FIG. 48 in which an internal housing chamber is filled with a hydrogel matrix 4842 or other fluidic conductive medium, which allows for diffusion and/or migration of insulin (represented by dots 4846). A lower boundary of the housing chamber includes a plurality of microneedles having hollow tips 4848 (two numbered) that open into the internal housing chamber. A plurality of non-conductive insulin delivery ducts or tubes 4851 fluidically connect the hydrogel matrix 4842 to an insulin delivery control system 4852, which may include an insulin reservoir and pump system as described above, among other components. Once delivered into the housing chamber, the insulin 4846 can then flow through the gel matrix 4842 and out of the hollow tips 4848 of the microneedles. The delivery system also includes a first electrical conductor 4854 and a plurality of second electrical conductors 4856 that are placed at an exterior tip of each microneedle as described above. The delivery system further includes an ultrasonic transducer 4853. Upon application of a suitable voltage between the electrical conductor 4854 and the plurality of second electrical conductors 4856, an electric field is created such that insulin molecules 4846 will migrate out of the needle tips 4848 and collect near the exterior electrode. The transport (migration and/or diffusion) of the insulin molecules 4846 is increased by the vibrations created by the ultrasonic transducer 4853. Thus, both iontophoresis and sonophoresis support the delivery process in the embodiment shown.

The preceding insulin delivery systems may be combined with any of the above-described glucose monitoring systems. However, when combining the above-described iontophoresis insulin delivery systems with an electrochemical glucose monitor system inside the same SPAID unit, the two systems will require electrical isolation from each other. Alternatively, the two systems may operate from different power supplies or batteries and have isolated electrical ground paths. This will prevent “cross-talk” between the electrochemical monitoring system and the iontophoresis-based system.

A potential issue with using hollow microneedles to penetrate the skin is the wound healing response from the body that may affect both insulin delivery as well as measurement of glucose or other analytes. These effects may be minimized by providing microneedles that produce as little trauma as possible so to limit the body response. Trauma can be reduced by proper selection of the size, shape and length of the needles. However, it may still be a challenge to minimize the healing response of the body at the site of insertion. In addition, it may be difficult to prevent body components from entering the needle tips, coagulating and forming a clot or blockage. Because of these issues, the use of coatings or anti-coagulants may be desired. These coatings can be applied to both the inner surface and the outer surface of the needles. The inner surface may be provided with coatings that reduce or prevent the formation of clotting or coagulation, allowing the needle pathway to remain open and free for insulin delivery and or analyte measurement in the case of a CGM or other analyte measurement system. The outer surface may be coated with wound healing suppressants that can slow the healing response and allow the site to remain viable for insulin delivery and analyte measurement for the duration of the SPAID 102 wear cycle. Exemplary drugs that act as wound healing suppressants include cytotoxic antineoplastic and immunosuppressive agents, corticosteroids, nonsteroidal antiinflammatory drugs (NSAIDs), and anticoagulants.

In addition to iontophoresis and sonophoresis, additives can be combined with the hydrogel used to transport insulin out of the microneedles as well as allow transport of glucose or other analytes into the microneedles. Beneficial additives for this purpose include surfactants. The surfactants increase the flow or transmission of the molecules through the hydrogel in the microneedles by effectively making the hydrogel pathways more “slippery”. Potential surfactants include docusate (dioctyl sodium sulfosuccinate), alkyl ether phosphates, benzalkaonium chloride (BAC), perfluorooctanesulfonate (PFOS), sodium dodecylsulfate (SDS), cationic surfactants (e.g., a homologous series of alkyl-N,N,N-trimethyammonium compounds with varying alkyl chain size), zwitterionic surfactants (e.g., N-dodecyl-N,N-dimethylammonium-propanesulfonate, DDPS), and nonionic surfactants (e.g., Triton X-100), among others.

In one possible installation method for an insulin delivery microneedle array, the user firmly places and presses the SPAID 102 base against the skin in the recommended body site locations. To further enhance the microneedle penetration into and through the stratum corneum an ultra-sonic transducer may be used to vibrate the body of the microneedle insulin delivery array temporarily and periodically. The ultrasonic vibrational energy can serve to agitate and further deepen the array to the desired maximum depth controlled by the needle length. This effect can be used in conjunction with pressure being applied by adhesives that can hold the SPAID 102 firmly to the skin and the skin pressure that was established during the initial installation by the user, which creates upward pressure force towards the base of the SPAID 102 and against the microneedle array. In this design, an adhesive is beneficially placed in close proximity to and all around the array and the array is mounted slightly proud of the SPAID base surface. In addition, the microneedle array 4402 may have a force applied from the interior of the housing onto the back of microneedle array 4402 such as from a spring, foam material or other force system to ensure and maintain proper downforce of microneedle array 4402 onto the surface of the skin.

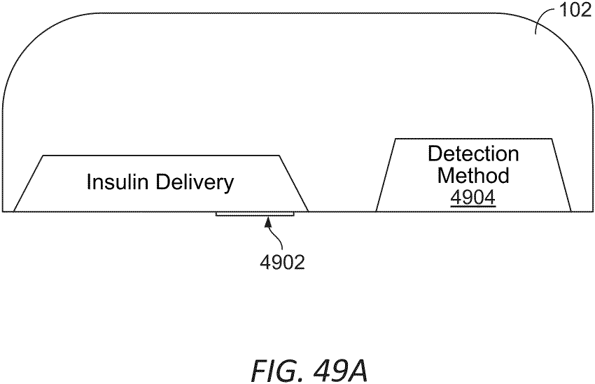

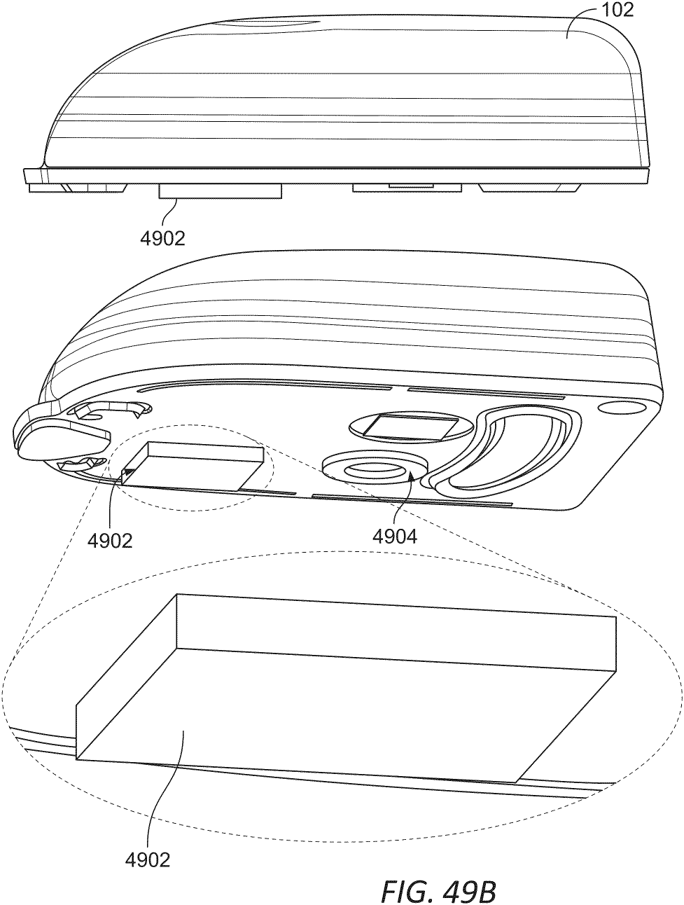

Another alternate embodiment of delivery patient interface 186 is shown in FIG. 49A and comprises a transdermal method of delivery comprising a transdermal patch 4902 mounted in the base of the housing of SPAID 102 to delivery insulin directly through the surface of the skin. Transdermal insulin delivery can be mated with any of the glucose sensing systems previous discussed herein. FIG. 49B shows a concept image of this embodiment, showing an enlarged view of the transdermal patch 4902 in the inset. Also shown is detection method 4904, which is shown here as a wireless communication antenna, such as a nearfield antenna, although any detection method may be used with the transdermal patch 4902 method of delivery, such as single needle sensing, microneedle sensing and or spectroscopy sensing.

As previously noted, the delivery of insulin can be performed by using a technique known as iontophoresis. The transport of insulin out of the housing of a SPAID 102 and into the body can be enhanced or accelerated by using iontophoresis to push or pull the insulin molecules through a hydrogel matrix from inside of the housing to the exterior housing of the SPAID 102 and into the skin. This is achieved by setting up a constant or varying electrical field using electrical conductors that are mounted between a location in the interior housing where the pump delivers insulin to the internal hydrogel matrix and the exterior bottom surface of the SPAID 102. Depending on the pH of the skin and the charge state of the insulin molecules, a specific voltage or sets of voltages with differing pulse profiles and or static levels can be employed. The driven electrical field will serve to push or pull the insulin molecules from inside the housing through the hydrogel matrix and toward the oppositive pole conductor(s). Since the opposite pole conductor(s) is mounted in the base of the SPAID 102 away from the insulin source, the insulin is forced to migrate through the skin towards the opposite pole conductor(s) which results in the insulin molecules being driven into and through the stratum corneum and into the deeper layers of the skin. Additionally, extra skin sensing conductors may be employed to sense the pH of the skin to provide feedback to the system and thus alter the field strength, wave shape, pulse time and or duration of the electric field to achieve the desired system performance. Once the insulin has been driven to the skin, the iontophoresis system can be turned off for a period time allowing for bodily diffusion to take over. The migration of insulin out of the housing into the body will decrease the delay from a time when an insulin delivery demand is received by the SPAID 102 to a time when the body receives the insulin. This system may not affect the quantity of insulin ultimately delivered, but it can control how quickly the measured insulin dose enters the skin.

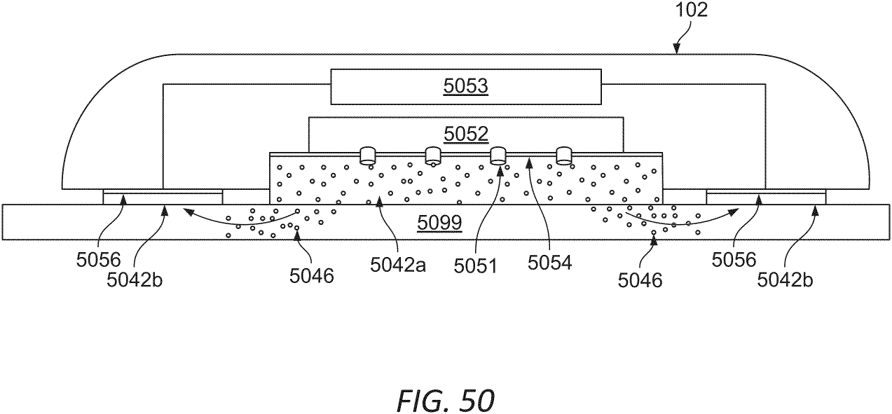

FIG. 50 illustrates the position of electrical conductors as part of the iontophoresis system of a SPAD 102. One of the conductors 5054 is positioned at the top of the housing as a continuous conductive surface with the non-conductive insulin delivery ducts or tubes 5051 placed in between. The insulin delivery ducts or tubes 5051 fluidically connect a first hydrogel matrix 5042a to an insulin delivery control system 5052, which may include an insulin reservoir and pump system as described above. The remainder of the housing is non-conductive except for opposite pole conductors 5056. Opposite pole conductors 5056 required to create the electric field are placed on the bottom surface of the SPAID housing to make a consistent low impedance connection to the surface of the skin 5099. A second hydrogel matrix 5042b is disposed over the opposite pole conductors 5056. Upon application of a suitable voltage between the conductor 5054 and the opposite pole conductors 5056 via iontophoresis control circuit 5053, an electric field is created such that insulin molecules 5046 will migrate in the direction of the opposite pole conductors 5056. Also depicted are insulin molecules 5046, which flow through and out of the hydrogel matrix 5042a and migrate through the skin 5099 towards the opposite pole conductors 5056. The system is configured to power off the electrical field periodically to allow the migrated insulin molecules to diffuse deeper into the skin for absorption by the body.

The electric field can be created using many different electrical voltage patterns. These patterns may be, for example, a sine, triangle, trapezoid, square, or pulsed waveform, or a constant DC level, or any desired waveform deemed effective at driving the insulin into the skin. In addition, any of the waveforms may be bipolar, AC, or a combination of AC and DC. The current used to create the electric field may be any level up to the maximum set by the medical device electrical safety standards. The voltage used may be any voltage limited to the medical device electrical standard required to force the desired current. The system may be driven at constant voltage, constant current, constant impedance, or any other mode useful to effectively drive the insulin into the skin. In addition, to prevent adverse skin reactions, the system may be capable of being turned off completely with the conductors being set to a high impedance level with respect to each other.

Several possible placements of the iontophoresis opposite pole conductors and the insulin source housing are described below. The design is not limited to these configurations. Care is taken to not place the CGM sensing area in between the insulin source pole conductor and opposite pole conductor to avoid the migration of insulin molecules across or near the CGM sensing area.

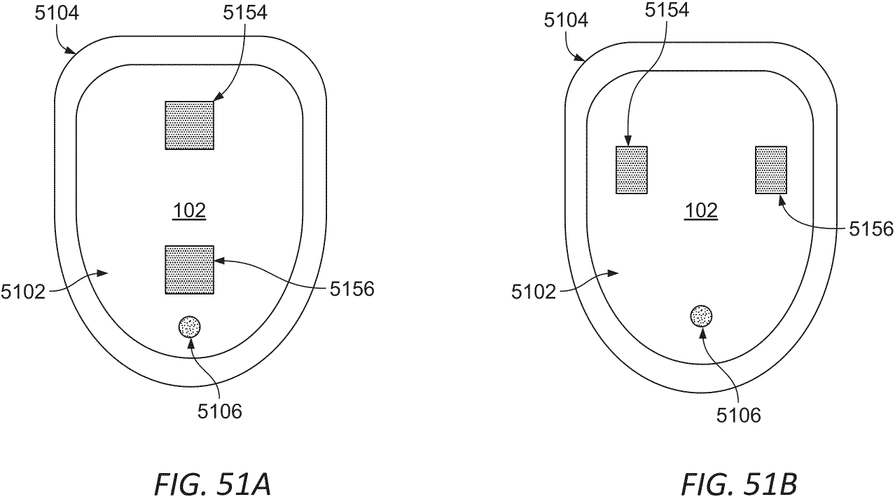

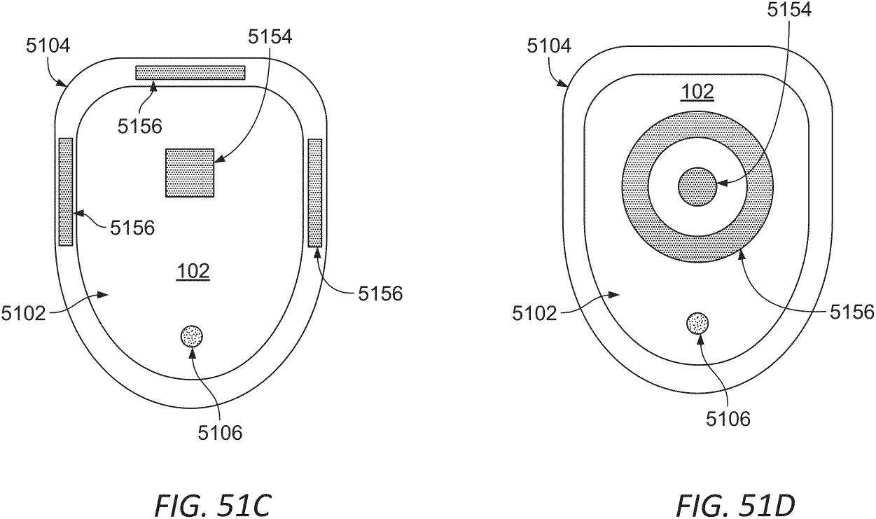

FIG. 51A shows a bottom view of SPAID 102 showing base 5102 which, when SPAID 102 is deployed, rests on the skin of the user. Adhesive 5104 serves to adhere SPAID 102 to the user’s skin. SPAID 102 includes a glucose sensing area 5106. This figure further illustrates one possible position for the pole conductors that are part of the iontophoresis system. In this implementation, one of the pole conductors (insulin delivery pole conductor 5154) resides at an upper portion of the base 5102. The remainder of the base 5102 is non-conductive except for the opposite pole conductor 5156, which is placed at a lower portion of the base 5102. A first variation of this embodiment is shown in FIG. 51B wherein the insulin delivery pole conductor 5154 and opposite pole conductor 5156 reside on the base 5102 in a side-by-side arrangement. FIG. 51C shows a third variation in which the insulin delivery pole conductor 5154 is disposed near the center of the base 5102 and multiple opposite pole conductors 5156 are disposed within the adhesive 5804 which adheres SPAID 102 to the skin of the user. Lastly, FIG. 51D shows yet another variation in which the insulin delivery pole conductor 5154 and the opposite pole conductor 5156 are disposed on n the base 3802 of SPAID 102, in which the opposite pole conductor 5156 is in ring-shaped form, and in which the insulin delivery pole conductor 5154 is in circular form and is disposed within a center of the ring-shaped opposite pole conductor 5156.

The delivery of insulin can be enhanced by using a technique known as electroporation. More particularly, transport of large molecules such as insulin out of the housing of the SPAID 102 and into the body can be enhanced by using electroporation to expand or open new pathways into the skin that are large enough to allow insulin molecules or other large molecules up to 10 kDa to pass through. This is achieved by using electrodes mounted in the base of the SPAID 102 that contact the skin surface directly, through a conductive hydrogel matrix or other conductive electrical media. The electroporation electrodes are capable of producing voltage pulses with a pulse width up to 2 ms in length. The pulses may be producible as often as once every 500 ms. The number of pulses in an electroporation session may be up to 300. Sessions may be repeatable as many times as required during the use of the SPAID 102 to maintain the pathway for insulin or other molecules to be delivered into the skin. The electroporation voltage may be up to 300 V DC to overcome the natural skin resistance. The current is generally limited to meeting all medical device electrical safety standards. Alternatively, low-frequency AC voltage may also be employed to open the pathways as well.

An additional consideration is the electrical isolation of a glucose-sensing system that is collocated with an insulin electroporation delivery system. The high voltage electrical pulses generated by the electroporation delivery system could damage or in the least effect the measurement values obtained from the glucose sensing system. For that reason, electrical isolation of the glucose measurement circuitry from the electroporation delivery system can be employed.

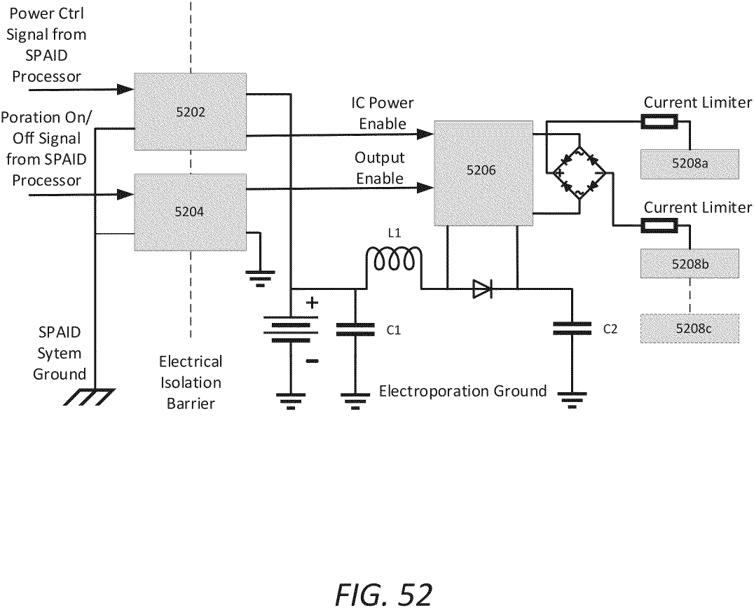

FIG. 52 is a schematic diagram showing an exemplary embodiment of a possible implementation of an isolated electroporation circuit. Electrical isolation of the glucose measurement system from the electroporation delivery system can be accomplished, in one embodiment, using opto-isolators 5202, 5204, or equivalent circuitry to isolate the power control signal and the poration on/off signal supplied by the SPAID processor from the high-voltage portion of the proration circuit. The high-voltage required for poration is generated by high-voltage booster 5206 and delivered to the user via electrodes 5208a and 5208b. In some embodiments, additional electrodes 5208c... may be connected together as needed.

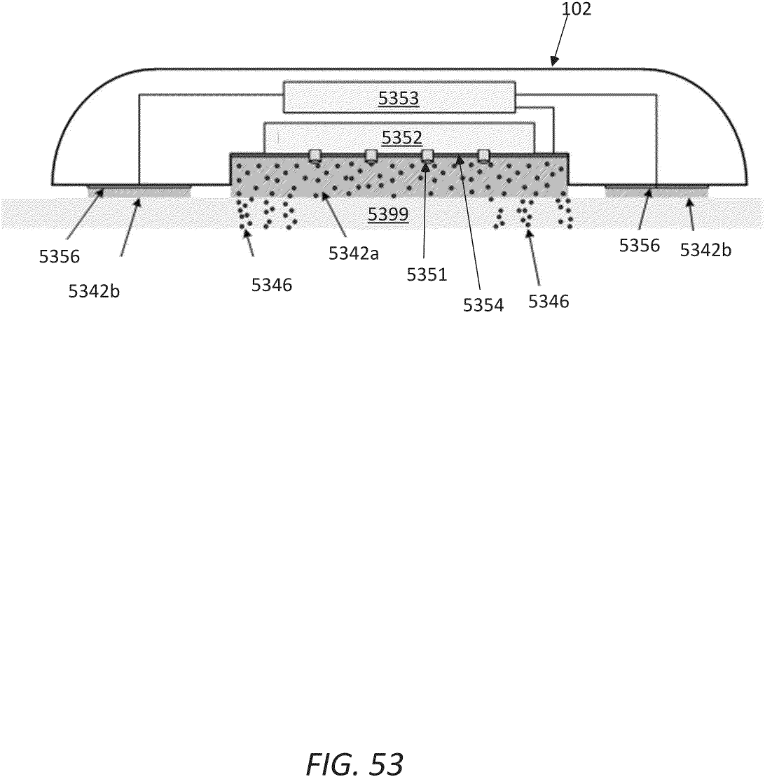

FIG. 53 illustrates one possible position of electroporation electrodes as part of an electroporation system of a SPAD 102. In this implementation, one of the conductors 5354 is positioned at the top of the housing as a continuous conductive surface with the non-conductive insulin delivery ducts or tubes 5351 placed in between. The insulin delivery ducts or tubes 5351 fluidically connect a first hydrogel matrix 5342a to an insulin delivery control system 5352, which may include an insulin reservoir and pump system as described above. The remainder of the housing is non-conductive except for opposite pole conductors 5356. Opposite pole conductors 5356 required to create the electric field are placed on the bottom surface of the SPAID housing to make a consistent low impedance connection to the surface of the skin 5399. A second hydrogel matrix 5342b is disposed over the opposite pole conductors 5356. Upon application of suitable electroporation voltage pules between the conductor 5354 and the opposite pole conductors 5356 via electrophoresis control circuit 5353, new pathways into the skin are expanded or opened allowing insulin molecules 5346 to penetrate the skin 5399. The figure depicts the flow of insulin molecules 5346 out of the pump housing and into the skin 5399 through the pathways opened by the electroporation system.

Alternatively, if a cannula or need-based CGM system is used as part of the SPAID 102, one of the isolated electroporation electrodes may be mounted on the cannula or needle. This would allow the electric current from the electroporation system to pass from inside the insulin delivery housing, through the skin and return through the needle or cannula electrode. This would eliminate the need for the exterior electrodes at the skin surface and may be beneficial for creating deeper, more conductive pathways into the skin.

Additionally, it can be observed that the possible implementations of the iontophoresis system are very similar to the implementation of the electroporation system. In this regard, the conductor(s) and the opposite pole conductor(s) of the electroporation system may be positioned relative to one and to a glucose sensing area on a SPAID base along the lines shown in FIGS. 51A-51D.

The use of both an iontophoresis system and an electroporation system could serve to further increase the delivery performance of a transdermal insulin delivery system. To utilize both systems, the electrodes can be implemented identically to each other. The electronic circuits used can also be quite similar. Both circuits can be isolated and operate from the main SPAID control processer through an isolation barrier. The control and timing of which systems are active and for what duration and performance level can all be controlled by the SPAID processor. One possible use scenario would be to use the electroporation system after the system is first installed by the user. Once the electroporation process is complete the electroporation system can be turned off and the iontophoresis system can be electrically connected internally to the same electrodes and activated. The iontophoresis system can be used periodically to increase the insulin flow. In addition, the iontophoresis system may be periodically turned off and the electroporation system is enabled to refresh or create new insulin conductive pathways. The timing of when these events occur and for how long can be controlled by the SPAID processor. Furthermore, the system can be configured to monitor the bodily response to insulin delivery based on CGM feedback and apply either or both of the systems with greater frequency or greater strength or duration to improve insulin delivery.

Transdermal delivery of insulin can be enhanced by using an ultrasonic sonophoresis system. The ultrasonic sonophoresis uses low-frequency ultrasonic waves to create cavitation bubbles both under the skin and on the surface when using an acoustic coupling medium. The bubbles created have a radius of approximately 150 um for 20 kHz ultrasound. During the sonophoresis process, the cavitation bubbles form and then collapse. If a bubble is formed on the outside skin surface in the acoustic coupling medium a distance equal to its radius from the skin, then the collapsing force from the bubble can create a micro jet into the stratum corneum, resulting in a perturbation of the skin. These skin perturbations can subsequently be utilized for delivery of insulin or another large molecule drug.

Ultrasonic sonophoresis can be achieved by the use of an ultrasonic transducer that is mounted on the inside of the housing near the insulin delivery tubing and in direct contact with the interior hydrogel matrix. The ultrasonic vibrations produced by the transducer travel through the hydrogel matrix and form cavitation bubbles on the exterior surface of the skin. The hydrogel may be formulated to have acoustic properties that match the acoustic impedance of the skin, thereby allowing for maximum energy transfer. The transducer may be operated in a range of 20-100 kHz and have a power transfer capability of up to 2 W per cm². The control system for the ultrasonic transducer may be provided with capability for setting the duty cycle of the device, the number of cycles, pulse duration, and overall runtime to achieve the desired sonophoresis skin perturbation effects.

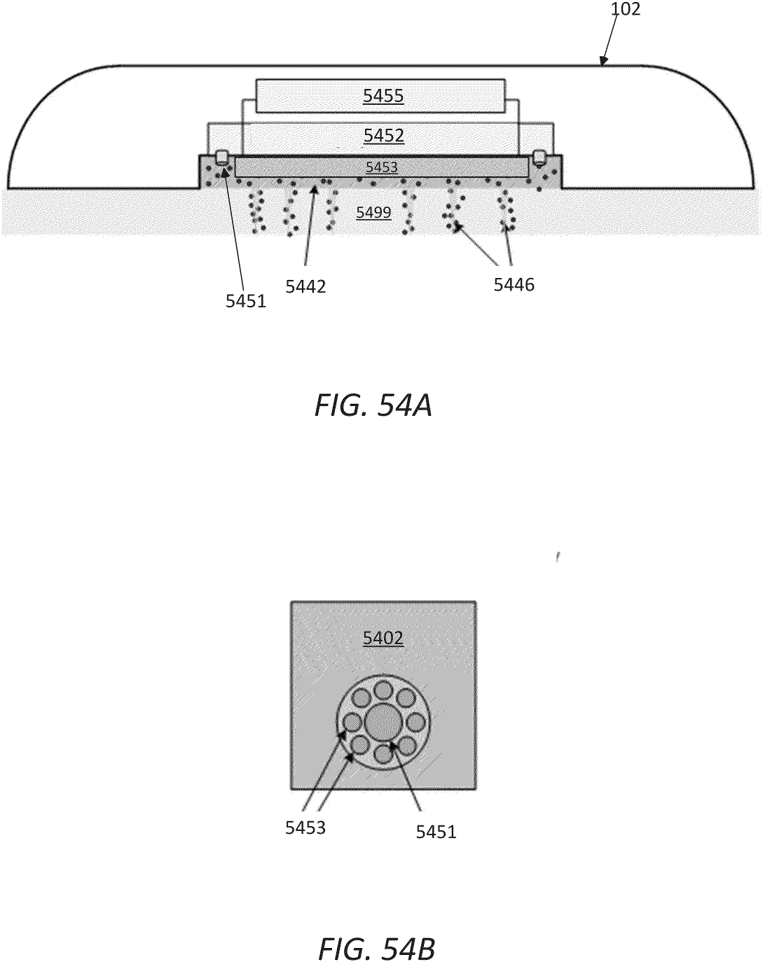

FIG. 54A illustrates a possible implementation of an ultrasonic sonophoresis skin perturbation system to support the delivery of insulin and other large molecules through the skin as part of a sonophoresis system of a SPAD 102. In this implementation, insulin delivery ducts or tubes 5451 fluidically connect a hydrogel matrix 5442 to an insulin delivery control system 5452, which may include an insulin reservoir and pump system as described above. Also shown are an ultrasonic transducer 5453, which is controlled by a sonophoresis control unit 5455. Upon operation of the transducer 5453, skin perturbations are created, which allow insulin molecules 5446 to penetrate the skin 5499.

Another possible layout of the sonophoresis system is illustrated in FIG. 54B which is bottom view of a SPAID 102 showing a portion of a base 5402 (not to scale) which, when SPAID 102 is deployed, rests on the skin of the user. In this figure, multiple ultrasonic transducers 5453 are placed in a ring around the periphery of an insulin delivery port 5451. A thin layer of acoustic impedance matched hydrogel may also be employed over the transducers 5453 to aid in the ultrasonic energy transfer to the skin.

The delivery of insulin through the skin may be also improved in any of the above embodiments by the use of chemical enhancers. When used in a hydrogel or other delivery mechanisms on the skin surface, the chemical enhancers diffuse into the lipid bilayer of the stratum corneum causing the disordering of the lipids in that layer allowing for nanometer sized opening or perturbations in that layer leading to enhanced transport and delivery of insulin or other large molecules in the deeper layers of the skin and absorption by the body. Examples of chemical enhancers, which may be topically administered in a wipe or may be encapsulated in a skin contacting hydrogel or lotion, include trypsin, castor oil, iodine, and oleic acid, among others.

Additionally, nanocarriers may be used as drug transport vehicles in any of the above embodiments to improve delivery of insulin or other large molecule through the skin. The insulin or large molecule would be encapsulated by the transport vehicle, which assists with the transport through the lipid bilayers in the stratum corneum. Transport vehicles include lipid-based vesicles, nanoparticles (CaCO₃), and nano emulsions, among others, which can greatly improve the transport of insulin through the skin.

The delivery of insulin can be performed by using a combination of any of the above-described enhancement techniques. For example, iontophoresis and electrophoresis systems could both utilize the same electrodes and hydrogel for contacting the skin. In addition, the bulk of the circuitry can be the same with isolation, power supply and controls being very similar, with the primary difference being in the magnitude of the electrical signals and their shapes and durations. The use of both systems could be quite beneficial with electrophoresis being used to open pathways in the skin and iontophoresis being used to exploit those pathways and further drive the transport of insulin and/or other large molecules. Furthermore, chemical enhancers could be added to the system along with the iontophoresis and electrophoresis system to further increase transport of insulin through the skin.

Lastly, the addition of sonophoresis into the system could complete and complement the use of the iontophoresis, electrophoresis and chemical enhancers to further open and maintain pathways into the skin.

Software related implementations of the techniques described herein may include, but are not limited to, firmware, application specific software, or any other type of computer readable instructions that may be executed by one or more processors. The computer-readable instructions may be provided via non-transitory computer-readable media. Hardware-related implementations of the techniques described herein may include but are not limited to, integrated circuits (ICs), application-specific ICs (ASICs), field-programmable arrays (FPGAs), and/or programmable logic devices (PLDs). In some examples, the techniques described herein, and/or any system or constituent component described herein may be implemented with a processor executing computer-readable instructions stored on one or more memory components.

To those skilled in the art to which the invention relates, many modifications and adaptations of the invention may be realized. Implementations provided herein, including sizes, shapes, ratings compositions and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A device comprising:
a housing;
one or more reservoirs disposed in the housing;
a fluid delivery mechanism for delivering a fluid from the one or more reservoirs to a user;
a sensing mechanism for sensing an analyte level of the user; and
a controller for analyzing the sensed analyte levels, determining a quantity and timing of delivery of the fluid and controlling the delivery mechanism to deliver the fluid to the user.

2. The device of claim 1 wherein the fluid delivery mechanism is a cannula subcutaneously inserted into the user.

3. The device of claim 1 further comprising:
a cannula configured with a 2-electrode sensor comprising a working electrode and a combination counter/reference electrode, or
a cannula configured with a 3-electrode sensor comprising one or more working electrodes, a reference electrode, and a counter electrode.

4. The device of claim 1 wherein the sensing mechanism uses one or more electrochemical cells.

5. The device of claim 4 wherein the electrochemical cells are in the form of one or more microneedle arrays.

6. The device of claim 4 further comprising:
an amplifier for amplifying signals from the one or more electrochemical cells;
a multiplexer for multiplexing the signals from the one or more electrochemical cells; and
an analog-to-digital converter for converting the analog signals from the one or more electrochemical cells to digital signals;
wherein software executing on the controller analyzes the digital signals to determine the analyte level.

7. The device of claim 1 wherein the fluid delivery mechanism comprises one or more microneedle arrays disposed on a base of the housing of the device.

8. The device of claim 1 wherein the fluid delivery mechanism comprises one or more transdermal patches disposed on a base of the housing of the device.

9. The device of claim 1 wherein the sensing mechanism uses one or more opto-fluorescent cells.

10. The device of claim 9 wherein each of the one or more opto-fluorescent cells comprises:
a light source, disposed in the housing of the device, the light source emitting an excitation light of a first particular wavelength;
one or more fluorescent elements which emits light of a second particular wavelength; and
a light detector, disposed in the housing of the device for detecting emitted light of the second particular wavelength.

11. The device of claim 10 wherein the one or more fluorescent elements are disposed on an external surface of the cannula and further wherein the cannula comprises:
an inner lumen used as the fluid delivery mechanism;
one or more light pipes coupled to the light source; and
one or more light pipes coupled to the light detector.

12. The device of claim 10 further comprising:
an amplifier for amplifying signals from the one or more opto-fluorescent cells;
a multiplexer for multiplexing the signals from the one or more opto-fluorescent cells; and
an analog-to-digital converter for converting the analog signals from the one or more opto-fluorescent cells to digital signals;
wherein software executing on the controller analyzes the digital signals to determine the analyte level.

13. The device of claim 1 wherein the sensing mechanism uses a spectrographic method utilizing a Ramen shift.

14. The device of claim 13 wherein the sensing mechanism comprises:
an excitation light source emitting light at a first wavelength; and
one or more Ramen detectors for detecting Ramen shifted light at wavelengths longer and shorter than the first wavelength.

15. The device of claim 14 wherein the excitation light source and the one or more Ramen detectors are disposed in the housing of the device.

16. The device of claim 14 further comprising:
a cannula having an inner light pipe for delivering the excitation light source and an outer light pipe for collecting the Ramen shifted light.

17. The device of claim 16 wherein the cannula is also used as the fluid delivery mechanism.

18. The device of claim 16 wherein the fluid delivery mechanism comprises a second cannula subcutaneously inserted into the user.

19. The device of claim 14 further comprising:
an amplifier for amplifying signals from the one or more Raman detectors;
a multiplexer for multiplexing the signals from the one or more Raman detectors; and
an analog-to-digital converter for converting the analog signals from the one or more Raman detectors to digital signals;
wherein software executing on the controller analyzes the digital signals to determine the analyte level.

20. The device of claim 1, wherein the sensing mechanism is configured to be sterilized in a sterilization process based on one or more sterilization parameters, and the sensing mechanism:
is constructed from or overlaid with a material selected based on its tolerance to the sterilization process;
is coated with one or more enzymes having at least one of a material or thickness that is selected based on the sterilization parameters; or
comprises a sacrificial coating selected based on the sterilization parameters.
